(19) United States
(12) Patent Application Publication
(10) Pub. No.: US 2026/0063633 A1
(43) Pub. Date: Mar. 5, 2026

(54) Methods and Reagents for Toxoplasma Infection Diagnosis

(71) Applicants: Rima McLeod, Chicago, IL (US); Denis Limonne, Lyon (FR); Francois Peyron, Lyon (FR); Kamal El-Bissati, Chicago, IL (US); Joe Lykins, Chicago, IL (US); Martine Wallon, Lyon (FR); Jorge Gomez-Marin, Armenia (CO); Ying Zhou, Chicago, IL (US)

(72) Inventors: Rima McLeod, Chicago, IL (US); Denis Limonne, Lyon (FR); Francois Peyron, Lyon (FR); Kamal El-Bissati, Chicago, IL (US); Joe Lykins, Chicago, IL (US); Martine Wallon, Lyon (FR); Jorge Gomez-Marin, Armenia (CO); Ying Zhou, Chicago, IL (US)

(21) Appl. No.: 19/342,645

(22) Filed: Sep. 28, 2025

Related U.S. Application Data
(63) Continuation-in-part of application No. 16/542,865, filed on Aug. 16, 2019.
(60) Provisional application No. 62/718,984, filed on Aug. 16, 2018.
(60) Provisional application No. 63/813,261, filed on May 28, 2025.

(51) Int. Cl. G01N 33/569; G01N 33/543

(52) U.S. Cl. CPC G01N 33/56905; G01N 33/54388; G01N 2333/45; G01N 2469/20

(57) ABSTRACT

Point-of-care (POC) testing for T. gondii infection can potentially address cost concerns and lead to better clinical outcomes through improved access to screening. This disclosure provides methods and compositions for whole blood POC testing to identify T. gondii infection with high sensitivity and specificity, obviating the need for venipuncture and sample processing infrastructure, making it an efficient, low-cost POC test.

### CROSS-REFERENCE TO RELATED APPLICATIONS

This application is a continuation-in-part application of U.S. patent application Ser. No. 16/542,865, filed on Aug. 16, 2019, which claims priority benefit of U.S. Provisional Patent Application Ser. No. 62/718,984 filed on Aug. 16, 2018. This application also claims priority benefit to U.S. Provisional Patent Application No. 63/813,261 filed on May 28, 2025. The disclosures of the aforementioned Provisional Patent Application Serial Nos. 62/718,984 and 63/813,261 and U.S. patent application Ser. No. 16/542,865 are hereby incorporated by reference in their entirety.

### GOVERNMENT SUPPORT

This invention was made with government support under AI027530 awarded by National Institutes of Health. The government has certain rights in the invention.

### BACKGROUND

It is now well established that monthly gestational serologic testing for acquisition of T. gondii infection for the first time during gestation facilitating prompt treatment can markedly improve patient outcomes and well-being. This can be utilized to permit prompt diagnosis and thereby treatment to prevent transmission to the fetus and also to ameliorate symptoms, resulting in saving life, cognition, motor function, sight, and dramatic benefit for improvement of quality of life. Toxoplasmosis causes substantial morbidity and mortality on a global scale. This disease can be severe. Vertical transmission from mother to fetus occurs from primary, acute infection during gestation. Congenital infection may result in chorioretinitis, hydrocephalus, epilepsy, and death. Serologic screening during gestation leads to early antenatal detection and rapid initiation of treatment with economic and patient outcome benefits. However, conventional serologic screening can be cost-prohibitive. Infrastructure, including electricity and equipment for sample processing, may be unavailable.

### SUMMARY

Point-of-care (POC) tests detecting T. gondii infection offer solutions, potentially addressing cost concerns and leading to better clinical outcomes through improved access to screening. Provided herein, is proof of principle for potential utility and widespread applicability in clinical settings for a lateral flow immunochromatography-based Toxoplasma IgG-IgM test using whole blood. This whole blood point-of-care test performed with high sensitivity and specificity, obviating the need for venipuncture and sample processing infrastructure, making an efficient, low-cost point-of-care test.

The ability to provide test results rapidly to a subject in need thereof is very important to impact outcomes of multiple conditions. Rapid tests to aid diagnosis and enable early detection of multiple diseases and physiologic conditions are being developed. Such tests are especially useful when they can be applied with self-testing and require little in the way of laboratory processing. Examples of point-of-care (POC) test devices in common use today include pregnancy and fertility tests. Development of diagnostic tests for infections that use POC testing are especially important in resource-poor settings; for this reason, POC testing has become a new goal to be achieved for infections such as Toxoplasma infection.

The inventors have surprisingly discovered that a whole blood test (using only about 20 to 30 microliters) can perform with high sensitivity and specificity, obviating the need for venipuncture (since the whole blood can be obtained, for example, by finger prick) and sample processing infrastructure (required to separate serum, etc. from whole blood), making the methods as disclosed herein an extremely efficient, low-cost, point of care (POC) test for Toxoplasma infection and treatment monitoring. Such POC testing has potential to significantly expand access to screening, for example, during gestation for this serious, potentially fatal infection, with spillover benefit in facilitating screening programs for other congenital infections and improvements in maternal fetal health and well-being and care.

As provided herein, demonstration of the high performance of this whole-blood POC test, and the test's strong functionality at the point-of-care, which has not been previously demonstrated, provides the proof of principle of its potential utility and widespread applicability in clinical settings. This new test also fulfills the World Health Organization criteria for the ideal POC test (Affordable, Sensitive, Specific, User-friendly, Rapid/robust, Equipment-free, and Deliverable to users; A.S.S.U.R.E.D.). Performance and interpretation of this POC test takes less than two minutes of operator time, with results available to be interpreted within 20-30 minutes, facilitating appropriate clinical intervention so that it is ideal for following those who are seronegative to detect seroconversion.

In one aspect, the invention provides a method for diagnosing a Toxoplasma infection in a subject at risk of a Toxoplasma infection, comprising contacting a whole blood sample from the subject to a sample well of a lateral flow immunochromatography device to diagnose a likelihood of the subject having a Toxoplasma infection.

In certain embodiments of the method for diagnosing a Toxoplasma infection, the lateral flow immunochromatography device comprises:
(a) providing a cassette, wherein the cassette comprises:
   (i) a nitrocellulose strip comprising a Toxoplasma antigen immobilized on a test band; and
   (ii) a conjugate pad in fluid communication with the nitrocellulose strip impregnated with test latex particles coupled with a Toxoplasma antigen; and
   (iii) a sample well in fluid communication with the conjugate pad;
(b) adding the whole blood sample to the sample well to initiate migration of the whole blood and the test latex particles along the conjugate pad, under conditions to promote binding of anti-Toxoplasma antibodies in the whole blood sample to the Toxoplasma antigen on the test latex particles to form positive complexes; and
(c) detecting an interaction between the positive complexes and the test band.

In certain embodiments, approximately 20 to 30 μL of the whole blood sample is added to the sample well. In some embodiments, the whole blood sample is obtained from a finger prick. In some embodiments, the nitrocellulose strip further comprises control latex particles that are optically distinguishable from the test latex particles, and wherein the conjugate pad of further comprises control antigens immobilized on a control band. In an embodiment, the test latex particles are black and the control latex particles are blue. In yet another embodiment, the control latex particles are impregnated with goat anti-rabbit antibodies, and wherein the control antigens comprise rabbit gamma immunoglobulins. In some embodiments, the anti-Toxoplasma antibodies in the whole blood sample are IgG and/or IgM antibodies.

In certain embodiments, the methods as disclosed herein comprise dispensing an eluting solution to the sample well, subsequent to the whole blood sample, and allowing migration of the whole blood sample and the eluting solution for 20-30 minutes before reading the test band and the control band of the device.

In certain embodiments, the Toxoplasma infection is the result of an infection by Toxoplasma gondii. In some embodiments the Toxoplasma infection is the result of an infection by Toxoplasma gondii in North America, Central America, South America, Hawaii, Caribbean Islands, France, Eastern Europe, North Africa, New Zealand, and Philippines. In some embodiments, the subject is a pregnant human or a human suspected of being pregnant.

In certain embodiments, the method has a sensitivity of at least about 90%, at least about 91%, at least about 92%, at least about 93%, at least about 94%, at least about 95%, at least about 96%, at least about 97%, at least about 98%, or at least about 99%.

In certain embodiments, the method has a specificity of at least about 90%, at least about 91%, at least about 92%, at least about 93%, at least about 94%, at least about 95%, at least about 96%, at least about 97%, at least about 98%, at least about 99% or at least about 100%.

In certain embodiments, the method further comprises treating those subjects that are identified as likely to have a Toxoplasma infection with a therapeutic for treating Toxoplasma infection. In some embodiments, the therapeutic is selected from the group consisting of: pyrimethamine, sulfadiazine, sulfapyrizine, sulfamethazine, sulfamerizine, azithromycin, clarithromycin, atovaquone, dapsone, cotrimoxazole, and combinations thereof.

In a second aspect, the invention provides a kit comprising a lateral flow immunochromatography device for diagnosing a Toxoplasma infection in a subject wherein the kit comprises:
(a) the lateral flow immunochromatography device, wherein the device comprises a cassette comprising:
   (i) a nitrocellulose strip on which are immobilized a Toxoplasma antigen in a test band and a control antigen in a control band;
   (ii) a conjugate pad in fluid communication with the nitrocellulose strip impregnated with test latex particles coupled with a Toxoplasma antigen and control latex particles coupled with a control antigen; and
   (iii) a sample well in fluid communication with the conjugate pad;
(b) eluting buffer.

In certain embodiments of the kit, the eluting buffer is in a 3 mL dropper bottle.

In some embodiments, the kit comprises: 60 lateral flow immunochromatography devices, wherein there are 6 bags, and each bag contain 10 devices and a desiccant packet; 3 dropper bottles with the eluting buffer, where each dropper bottle comprises 3 mL of eluting buffer; and instructions for use. In yet another embodiment, the kit comprises: lancets for fingertip whole blood sampling; capillary tubes for collecting the whole blood sample; and a timer.

The methods and kits described herein provide for more effective diagnosis and/or treatment of life-threatening or irreversibly debilitating human disease or conditions resulting from infection from T. gondii. The methods and kits described are being considered for the FDA Breakthrough Devices Program and meet the WHO ASSURED criteria. Currently, there are no approved or cleared alternatives for whole blood testing from a finger stick (i.e. using about 20 to L of whole blood), and the methods and kits as described herein provide significant advantages over existing approved and/or cleared alternatives. Finally, the methods and kits as described herein are in the best interest of the patients.

### BRIEF DESCRIPTION OF THE DRAWINGS

FIG. 1A: is a schematic of the study participant composition in United States and Morocco. One seropositive individual was tested via point-of-care (POC) test twice, but is included in this number only once. Nineteen (19) pregnant women were tested a total of three times each as part of a pilot gestational screening program. All were seronegative. Volunteers learned of the study through word of mouth sometimes when friends or family members knew of an affected child and asked to participate or were formally recruited in an obstetrical practice. No one complained or mentioned medical problems of any type other than as related to toxoplasmosis among those who were seropositive from the NCCCTS or pregnancy, although there was no health questionnaire or physical examination as part of this study.

FIG. 1B: Performing the Toxoplasma ICT IgG±IgM Test. The site of the finger-stick is cleansed with an alcohol wipe and pricked with a lancet. Blood is collected via capillary tube and applied to the test kit. Four drops of eluent are then applied. The test can be interpreted in 20 to 30 minutes. The black arrow adjacent to the ‘T’ indicates the presence of antibodies against T. gondii. NCCCTS, National Collaborative Chicago-Based Congenital Toxoplasmosis Study; POC, point of care; UofC, University of Chicago.

FIG. 2: Shows an exemplary device with an immunochromatographic test strip included in a cassette. From left to right, the strip is composed of a cellulose pad called “sample pad” (1) where are dispensed the sample and the eluent in the “sample well” (2). Then there is a fiberglass pad, the “conjugate pad” (3) loaded with blue latex particles coated with anti-rabbit goat antibodies and black latex particles coated with Toxoplasma antigens (obtained from in vivo culture). After the conjugate pad, there is a nitrocellulose sheet (4) on which are dispensed two lines of reagent, one of Toxoplasma antigens, the “T”, “test line” (5) and one of rabbit antibodies the “C”, “control line” (6). If anti-Toxoplasma antibodies are present in the sample, the test line (“T”) will appear in black. The control line (“C”) will appear regardless of the presence of anti-Toxoplasma antibodies in the sample. The absence of the control line demonstrates the failure of the test and results can't be interpreted. Finally, a second cellulose pad at the right-end, the “absorbent pad” (7) completes the strip.

FIG. 3A: Shows exemplary devices showing both negative (left) and positive (right) results.

FIG. 3B: Shows exemplary devices showing both negative (top) and positive (bottom) results that could be included, for example, in training and instruction materials with the device.

FIG. 4A-4E show the “Roadmap” and Context for Study 1 Performed herein.

FIG. 5A-5F show the high performance of ICT. A. Clinical Feasibility Trial. Solid symbols represent positive predicate test, open circles represent negative predicate test. Small black dot indicates false positive predicate test. Solid colored symbols indicate a positive test. Open symbols indicate negative test result. Each of the three testers is indicated by a different color in this figure* False positive IgM predicate test for seropositive person. B. ICT is negative with 32 false positive standard tests in Lyon. C. ICT is negative with false positive standard tests in Paris, Blue IgG, green IgM. D. ADBIO, a USA test, suffers from false negative and positive IgM results. E. ICT detects acute infections with positive IgG and IgM in Colombia. F LOD Quick Instructions and Limit of Detection Study 6 Results. Box 3 contains Design and detail of methods and results. Quick instructions are able to teach 9 testers, 3 physicians, 3 medical students and 3 nurses to use test accurately with 7 samples that are negative and 7 samples at the limits of detection. Details of each study are also in Tables and Figures as follows in parentheses: FIG. 2A (Study 1, FIG. 1A-1E, Table 1); B. (Study 2a, Table 1); C. (Study 2b and Box 2); D. (Study 10, Table 5); E. (Study 7, Table 5); F. Box 3 and its legend provide detail for FIG. 2F (Study 6). Limits of Detection and Quick Information, Box 3-Part A is the actual Quick Information (QI) provided, Photograph of hand and kit from [23] open access license. Box 3 Part B shows the study design, Box 3 Part C shows examples of test results for two testers, Box 3D shows the tabulated results of all the readers in real time and of the photographs). Box 3 Legend provides Study 6's structure and results. In this study we demonstrate the ease in learning to use a simple, carefully designed test description and that these specific instructions which were prepared in accordance with FDA and CLIA requirements and review (A), can function as an effective teaching tool (B, C). This study 6 is also pertinent to demonstrating repeatability and reproducibility of this test. In (B), whole blood was tested at the defined limits of detection of the sample, with 14 negative or positive samples. Positive or negative status is blinded for these 9 independent tester/readers plus additional blinded reader. For this study, these 9 testers were without other training with the ICT and worked in 3 different locations. (B, C). Performance was perfect for each of them after reading this page of instructions (B, C). This is consonant with extensive and rigorous testing of use of this ICT in a variety of USA settings, as well as in other countries in many settings with minimal instruction. Detail of objective, methods, data content of Study 6 also is in the accompanying Box. As outlined in the Box, Study 6 shown in 2F, demonstrates that it is easy for physicians, physicians in training, and nurses to learn to use a simple carefully designed description (Quick Information, QI) shown in (A). The QI is about use of the ICT. This study and its results demonstrate that this QI is an effective teaching tool. Also, we find that results with the ICT are robust, repeatable, reproducible, sensitive and specific in multiple settings. Box 2 and Tables 1, 2 and 3 and Box 3 and Perspectives section of Discussion contain additional pertinent information and data.

FIG. 6A-6C show A. Graphic showing monthly test method. Use of ICT in NRL as considered originally in and modified from our earlier work (33). Help refers to seeking expert guidance and reference laboratory assistance. B. Role of ICT in this algorithm shown diagrammatically. C. Representative example of ICT detecting very early seroconversion using sera originally stored for another purpose tested retrospectively, showing contrast with false positives. First pregnancy by IVF. The testing was all done retrospectively after positive IgG, and IgM at ˜12-14 weeks gestation was discovered. At this time the ICT was an experimental research laboratory test done prior to studies performed for consideration of FDA clearance, CLIA waiver process and not for clinical care. However, they demonstrated, in this unusual circumstance, sensitivity early in this true seroconversion. This was while the Sabin Feldman Dye test that detects IgG directed against T. gondii and other Palo Alto Specialty laboratory tests including the IgM ELISAs were negative (antedating the time that they later became positive documenting seroconversion). Details with dates were as follows: December 2018. Began planning for IVF-sera Toxoplasma gondii antibody negative (Neg). March 2019. IVF embryos harvested cryopreserved-sera Toxoplasma gondii antibody negative. March 2019. Patient traveled to New Zealand returned end of March to USA. April 6—Patient sera obtained pre-transfer of embryos. Palo Alto reported negative g and m but there was for the first-time background in IgM ELISA, (˜0.2, cut off ˜2 for an adult), ENZO lab at NYU hospital —commercial lab reported T. gondii specific IgM positive. Since the commercial IgM was not considered reliable because of known false positives, the serum sample was sent to Palo Alto where this was found to be negative (neg). April 8-Serum samples sent to University of Chicago Research laboratory, were tested retrospectively at a later time. ICT was weakly, but clearly positive *; Note this was pre-implantation of the embryo and IVF was in March before infection. This test is binary and not quantitative. April 9—Embryo transferred to woman. April 16-Sera hormones showed embryo implanted. ICT from this time was weakly positive when tested later, and was a little stronger than in the first sample. Five observers confirmed these readings. This was all with the same lot of the ICT. April 22—ICT a little stronger positive. Dye test 1.64, M˜9, Avidity low, AC/HS acute. Subsequent sera also positive with significant rise in dye test titer and acute patterns for other tests. Pregnant woman began spiramycin at ˜12 weeks gestation when the first IgG dye test, and IgM ELISA were positive. Other sera collected and tested retrospectively. Amniocentesis at 18 weeks, tested at Remington Specialty Laboratory. PCR was negative. All obstetrical ultrasounds were normal. Infant was uninfected. Abbreviations: neg is negative, + is positive, nd is not done. SFDT is Sabin Feldman Dye Test. IVF, in vitro fertilization. “wild screening” is a colloquial term referring to non-systematic testing.

FIG. 7A-7C show feasibility and acceptability of monthly screening in U.S. Academic obstetric practice. Abbreviations in A. “neg” indicates negative results in tests. Note congruence of ICT and Reference Laboratory test conclusions. * Elective termination secondary to fetal anomalies, ** Spontaneous abortion, *** Elected to leave the study after first test time due to traditional beliefs regarding having blood drawn. “md” indicates a missed appointment, as patient did not attend her regularly scheduled appointment. Came at alternative time and we did not connect for testing and/or survey. “n/a” indicates not available. “L&D” indicates that the patient's monthly test was missed due to concern for premature labor, which resulted in a visit to the emergency department and then labor and delivery. “E” indicates an equivocal result, in which a barely visible band appeared which was not reproducible upon photographing the test. Per manufacturer instructions, this test was interpreted as negative. +Survey sent later, not included on graph as was not at initially planned time of six-week postpartum visita. A. Times and results of monthly screening for each participant. B. Survey questions and Likert scale. n the survey, number responses were considered as follows: strongly agree was 5, 4 was agree, 3 somewhat agree, 2 was somewhat disagree, 1 was strongly disagree C. Results of satisfaction survey for 14 participants. Each respondent's answer is represented by the circles in the figure. The mean is indicated by the horizontal line, with error bars indicating standard deviation. In response to questions 1 and 4, 13/14 respondents indicated that they “strongly agree” that they would pursue testing for T. gondii in future pregnancies with POC testing, and that knowledge of T. gondii is important for pregnant women. Results were more mixed if testing required venipuncture, as indicated by the responses to questions 2 and 3, but most agreed that the test was important, would have it again in a subsequent pregnancy, and would recommend this to family and friends. It was noteworthy that other family members such as fathers of the fetus asked to be tested, relevant to the possibility of retinal disease. Right panel showed results after the time of the 6 week post-partum visit. ‘We did not conduct a formal survey at the time; however, we began with two providers, and other providers in the practice asked to join with their patients. Providers continued in the further analysis of the ICT as it moved toward clearance and waiver.

FIGS. 8A and 8B show location of seropositive persons in Cincinnati and associated demographic factors such as socioeconomic status, maximum educational level achieved, pet ownership, and ethnicity. Sera were collected between 2017 and 2019. The low prevalence of seropositivity did not allow testing for clustering by any known risk factors for infection, including proximity to watersheds associated with sewage water run-off. None of the sociodemographic parameters, neighborhood deprivation, nor residential latitude and longitude measures achieved statistical significance. The measures of neighborhood deprivation are indicated by the color of the symbols.

Summary of purpose and results of each study in a Roadmap of the Studies. Flow diagram of studies that provide

FIG. 9 shows a roadmap to step changes leading to paradigm shift in prevention of congenital toxoplasmosis.

FIG. 10 shows an overview in the context of other work. Top presents context toward introducing screening in a global initiative. Red font (light grey in black and white) shows studies in provisional application.

FIG. 11. USA anonymized sera (no whole blood) tested in Bichat Hospital in Paris with a variety of tests (Abraham et al) including Biomerieux TXC with near perfect correlation with ICT result. These were obtained in the USA from samples sent for syphilis testing to be discarded after testing in the University of Chicago Clinical Laboratory in April 2019. Testing in that month was performed on 2019 person sera (859 females and 410 males). 272 were pregnant. 587 were not pregnant. Age stratification is shown. No one was pregnant below 20 years. Violet (top) males; Green (bottom), females.

Understanding data concerning the use of simple inexpensive diagnostic ICT.

Demographics of USA patients' samples during the time frame of those tested in Abraham et al paper were obtained. Demographics of persons who provided serum to be tested in the month that the samples provided to the Paris Bichat laboratory for testing are shown in detailed examples below for TXC predicate test.

New data documenting concordance of USA samples tested using the Biorad Bioplex 2020 with Abbott Architect IgG assays in Lyon French reference laboratory. Residual samples from the USA Clinical Trial (Clinical Trial.gov) were then also tested in the Lyon Reference laboratory using the Architect IgG with perfect concordance with the BioRadBioplex 2020 IgG and thus allowing direct meaningful and uniform comparison with the French data in accordance with the FDA requirement that a single uniform test be utilized.

FIG. 12 shows a summary of Line data prepared for FDA 510 k Clearance Dual CLIA Waiver.

FIG. 13 show tests used for multiple comparisons with ICT in many different demographics and locales as shown.

FIG. 14 shows examples of infections in older persons known to be infected almost 50 years earlier still positive in ICT, including person known to be infected with RH Remington strain which has known sequence. And person known to have isolated IgG in low titer in dye test in mid 1970s.

Example of positive test for a person infected 50 years with RH strain of T. gondii in needle stick laboratory accident followed by seroconversion.

Example of positive test for a person who was a low positive control for the Sabin Feldman dye test in the USA Palo Alto Medical Research Foundation Reference laboratory in 1974˜50 years later.

Example of positive test for a person who was infected congenitally on the USA East coast 18 years earlier.

FIG. 15 shows in the top panel an example of a positive test for a mother of a person who was infected congenitally 14 years earlier shown after ventriculoperitoneal shunt revision with his younger brother. The diagnosis for this child was missed for the month he was in an elite hospital given corticosteroids while he developed massive hydrocephalus despite many signs of congenital toxoplasmosis such as thrombocytopenia that were missed throughout that month.

Systematic serologic testing would have prevented this. Next panel again illustrates irreversible consequences of delayed diagnosis and absence of screening program in the USA that ICT could help to prevent. Another patient not shown, 38 year old G4P2 nurse, mother who presented with a febrile illness with missed diagnosis and baby still born infant at 32 weeks gestation. Placenta showed villitis histologically. Immunostaining of placenta showed T. gondii. It was not possible for her to obtain serologic tests for T. gondii at the time, delayed immunostaining. She was told she could not have toxoplasmosis because she did not own a cat. Image of parasite in placenta immunostained. Maternal serologies confirmed acute infection in reference laboratory. Consumption of rare venison was likely source of infection. Image of T. gondii immunostaining of placenta. Next image shows preventable Hydrocephalus in infant of mother who had normal fetal ultrasound at 28 weeks gestation, no illness, optimal prenatal care but no screening for Toxoplasma seroconversion. CSF protein >1000 mg/dl, in an apparent increased number of cases in Nashville, Tennessee in April, 2025. Retinal lesions and vitritis overlying them central and severe in one eye, peripheral in other. Shunts placed for hydrocephalus, shown before surgery. Next image shows an example of preventable Mother developed gestational diabetes, pulmonary disease at term, infant in intensive care unit with heart failure and treated with corticosteroids, Retinal disease missed and then importance unrecognized and untreated. Diagnosis missed for 6 months with delayed treatment in 2024. Epiretinal membrane is shown. Compounding pharmacies in New York City could not receive reimbursement for the cost of sulfadiazine as a policy from insurers was to reimburse at a rate substantially below costs. Pyrimethamine costs were reimbursed although remain quite high. These problems make obtaining care with adequate health care insurance coverage untenable in New York City at the present time. Next image shows. t. Lower panel. Congenitally infected infant born prematurely with hydrocephalus and retinal involvement. Next image acute retinitis. Next image large foveal scar. Next imaged.

Ferguson, scanning electron micrograph of the cyst characteristic of chronic infection in the brain of ⅓ of the world population, incurable at present and not without consequences as described in text. This is a. source of recrudescent infection ICT could help enable prompt treatment to prevent harmful consequences.

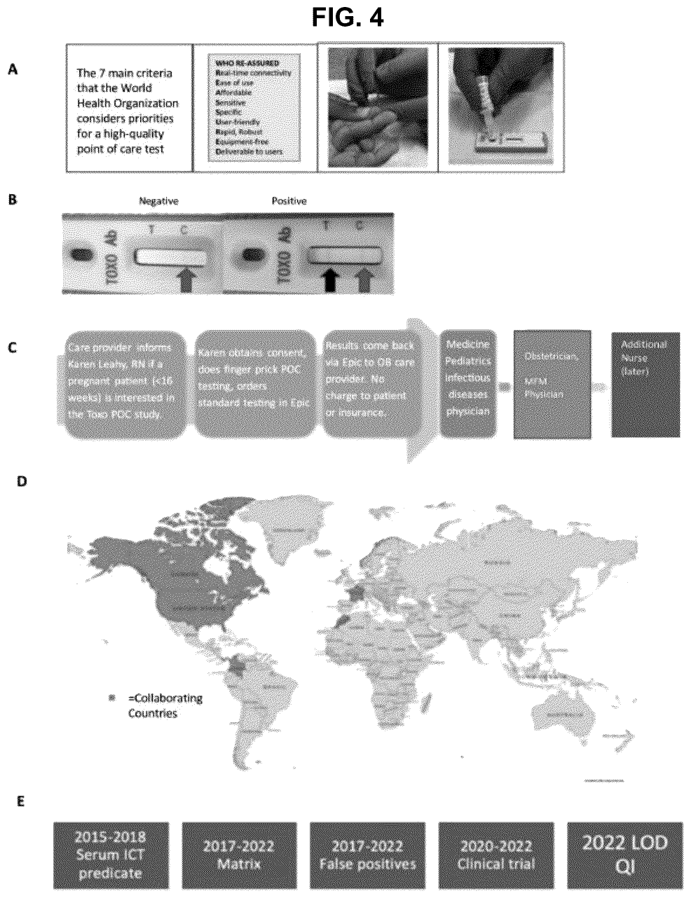

FIG. 16 shows a 7 year old child with Toxoplasma pneumonia, and encephalitis following stem cell transplant for osteogenic sarcoma who failed TMP/SMX treatment with persistent parasitemia. More Detailed Clinical Course. J-A- Clinical course timeline, with notes as to major events during the hospitalization of this child, as well as an indication of the resolution of laboratory evidence of infection, as shown by blood PCR for T. gondii, which resolved shortly after the initiation of appropriate anti-parasitic medication with pyrimethamine, sulfadiazine, and leucovorin. J-B-Axial FLAIR images through the mid cerebellum (top row) and periventricular white matter at the level of the body of the lateral ventricles (bottom row). A small FLAIR hyperintense lesion in the left cerebellum (green arrow) develops on Day +56 but gradually decreases over time. The relatively symmetric ill-defined FLAIR hyperintensity within the periventricular white matter diffusely (red arrows) is most conspicuous in the frontal and parietal lobes, and appears similar from Day +40 to Day +78, but significantly progresses on the Day +102 exam, where the extent becomes much more peripheral, to now involve the frontoparietal subcortical white matter. J-C-The findings on her chest CT are a more classical, dramatic presentation of toxoplasmosis. Starting on Day +42, her imaging demonstrated lung infiltrates with bilateral pulmonary opacities and diffuse ground glass opacities. The image in FIG. 4a was obtained just prior to her respiratory collapse. The lung pathology seen on imaging was consistent with worsening of her respiratory status requiring transfer to the intensive care unit. Despite treatment with TMP/SMX and clindamycin, her disease continued to progress (Figure I-B). Along with her altered mental status, she had with acute respiratory insufficiency requiring intubation shortly after the image in Figure I-B. After appropriate treatment and immune reconstitution, she greatly improved by Day +196 (Figure I-C). At this point she was ready for hospital discharge, playful and interactive. Effect of antimicrobial agents on tachyzoites. The ICT is useful at many steps in management in this setting including pre transplant, during clinical course, along with conventional serologies because of its WHO ASSURED properties.

FIG. 17 shows prevalence and payor mix for women of childbearing age status In these studies we compared selective spatial prevalence of Toxoplasmosis in the USA and its association with socioeconomic in this snapshot of seroprevalence in the USA. In this study we predominantly detected with ICT and other tests in epidemiologic studies, is not always associated with source laboratory payor mix or socioeconomic status of patient. Some of the data in the text are not added to this poster but the conclusions were informative, consonant with earlier observations of the NCCCTS, different from NHANES analyses and thus striking and therefore included herein.

FIG. 18. shows results for Apolipoprotein A measurements in acutely infected pregnant women and severely infected infants. Apolipoprotein A is a biomarker that can be used in conjunction with the ICT test described herein in various embodiments, and can be used as a test, or multiplex test, that when elevated indicates acute infection in adults. However, in congenitally infected infants, or recrudescent disease in young children, that are severely involved Apolipoprotein A level is diminished. Another example where the ICT could be used, in conjunction with another to test is the antibody to sporozoites. Such antibody to sporozoites is present for only the duration of the half life of the antibody over time. This is because it is initially formed only at the time of a point source exposure without sustained presence of the antigen from this life cycle stage of Toxoplasma. An additional example of combining the ICT with another test that provides information about the infection is the use of serotyping assays to determine the genetic identity of the parasite. Further in a similar sample of whole blood, the parasite sequences can be obtained for more detailed analysis of parasite genotype and pathogenicity factors as in the patient described in FIG. 16. Further because multiple infections appear to function in an additive matter to increase cognitive loss in older adults the ICT can be used in conjunction with single or multiplexed tests for other pathogens.

Further the ICT is an important part of a tool kit to build systems to optimize healthcare for Toxoplasma infection in its many manifestations and consequences. These include differentiating maternal and infant antibodies at birth with comparative western blots of sera of mother and infant when the ICT identifies an infant with antibody to Toxoplasma. In addition, this tool kit, including the ICT, can be used for the development of preventive and curative vaccines, and medicines both small molecule and antisense.

### DETAILED DESCRIPTION

All references cited are herein incorporated by reference in their entirety.

Terms used in the claims and specification are defined as set forth below unless otherwise specified. In the case of direct conflict with a term used in a parent provisional patent application, the term used in the instant specification shall control.

As used herein, the singular forms “a”, “an” and “the” include plural referents unless the context clearly dictates otherwise. “And” as used herein is interchangeably used with “or” unless expressly stated otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, the term “about” is understood as within a range of normal tolerance in the art, for example within 2 standard deviations of the mean. The term “about” is understood to refer to within 5%, 10%, 9%, 8%, 7%, 6%, 5%, 4%, 3%, 2%, 1%, 0.5%, 0.1%, 0.05%, or 0.01% of the stated value. When particular values or compositions are provided in the application and claims, unless otherwise stated, the meaning of “about” or “comprising essentially of” should be assumed to be within an acceptable error range for that particular value or composition.

All embodiments disclosed herein can be used in combination, unless the context clearly dictates otherwise.

In one aspect, this disclosure provides methods for diagnosing a Toxoplasma infection in a subject at risk of a Toxoplasma infection, comprising contacting a whole blood sample from the subject to a sample well of a lateral flow immunochromatography device to diagnose a likelihood of the subject having a Toxoplasma infection. In some embodiments, the about 20 to about 30 μL of the whole blood sample is added to the sample well. In some embodiments, the whole blood sample is obtained from a finger prick.

In certain embodiments, the method is used to diagnose whether the subject is suffering from a T. gondii infection. In such an embodiment, the subject is suspected of suffering from a T. gondii infection based on the presence of one or more symptoms, and the methods can be used to assist in providing a more definitive diagnostic, along with all other factors to be considered by attending medical personnel. The methods may be used to determine infection by any wild type, mutant, or recombinant Toxoplasma strain. In an embodiment, the Toxoplasma comprises any wild type, mutant, or recombinant Toxoplasma gondii strain. For example, the methods and kits as disclosed herein may be used to determine infection by genetically polymorphic strains from Argentina, Austria, Colombia, France, Germany, Lithuania, Morocco, Panama, Uruguay, and/or the United States. In certain embodiments, the Toxoplasma infection is the result of an infection by Toxoplasma gondii in North America, Central America, South America, Hawaii, Caribbean Islands, France, Eastern Europe, North Africa, New Zealand, and Philippines.

Additionally, in some embodiments, the methods and kits provided herein can be used to detect an acute infection. In certain embodiments, the methods and kits provided herein can be used to detect a chronic infection.

In certain embodiments of the methods disclosed herein, the lateral flow immunochromatography device comprises:
(a) providing a cassette, wherein the cassette comprises:
   (i) a nitrocellulose strip comprising a Toxoplasma antigen immobilized on a test band; and
   (ii) a conjugate pad in fluid communication with the nitrocellulose strip impregnated with test latex particles coupled with a Toxoplasma antigen; and
   (iii) a sample well in fluid communication with the conjugate pad;
(b) adding the whole blood sample to the sample well to initiate migration of the whole blood and the test latex particles along the conjugate pad, under conditions to promote binding of anti-Toxoplasma antibodies in the whole blood sample to the Toxoplasma antigen on the test latex particles to form positive complexes; and
(c) detecting an interaction between the positive complexes and the test band.

In one embodiment, the test is run by successively dispensing the whole blood sample and an eluting solution (called the eluent) in the “sample well” of the cassette. Adding the eluent starts the concomitant migration (chromatography) of the whole blood sample and the test latex particles and the control latex particles. This migration is completed in 20-30 minutes. If anti-Toxoplasma antibodies (IgG and/or IgM) are present in the whole blood sample, then a complex is formed between the test latex particles coupled to Toxoplasma antigens and the patient's antibodies which is then captured by the test band. For detection, the latex test particles may be colored (such as black), in which case results in the appearance of a black line at the T-band if the test is positive. The control latex particles may be optically distinguishable from the test latex particles (for example, blue colored latex particles) such that direct capture of the control latex particles by the control antigens on the nitrocellulose strip C-band results in the appearance of a blue line, meaning that the chromatography performed well.

The binding of black latex particles to the test line is done by the bivalent property of antibodies (or pentavalence of IgM), as the same antigen is used in both the coating of latex test particles and the test line. In some embodiments, the methods and kits provided herein can be used to detect false positive IgM antibodies from the subject. The binding of blue latex particles to the control line is done by the direct antibody-antigen reaction between the anti-rabbit goat antibodies (that act as antibodies) and the rabbit antibodies (that act as antigens).

As used herein, the term “subject” refers to any animal, including mammals, but not limited to, mice, rats, other rodents, rabbits, dogs, cats, swine, cattle, sheep, horses, or primates, and humans. In some embodiments, the subject is pregnant. In certain embodiments, the subject is a human. In an embodiment, the subject is a pregnant human. In certain embodiments, the pregnant human is tested within the first two weeks of pregnancy. In some embodiments, the pregnant human is tested before the third week of pregnancy. In some embodiments, the pregnant human is tested before the fourth week of pregnancy. In some embodiments, the pregnant human is tested before the fifth week of pregnancy. In some embodiments, the pregnant human is tested before the sixth week of pregnancy. In some embodiments, the pregnant human is tested before the seventh week of pregnancy. In some embodiments, the pregnant human is tested before the eighth week of pregnancy. In some embodiments, the pregnant human is tested before the eighth week of pregnancy. In some embodiments, the pregnant human is tested before the eighth week of pregnancy. In some embodiments, the pregnant human is tested before the eighth week of pregnancy. In some embodiments, the pregnant human is tested before the eighth week of pregnancy. In some embodiments, the pregnant human is tested before the eighth week of pregnancy. In some embodiments, the pregnant human is tested before the tenth week of pregnancy. In some embodiments, the pregnant human is tested before the twelfth week of pregnancy. In some embodiments, the pregnant human is tested before the fourteenth week of pregnancy. In some embodiments, the pregnant human is tested before the sixteenth week of pregnancy. In some embodiments, the pregnant human is tested before the eighteenth week of pregnancy. In some embodiments, the pregnant human is tested before the twentieth week of pregnancy. In some embodiments, the pregnant human is tested before the twenty-second week of pregnancy. In some embodiments, the pregnant human is tested before the twenty-fourth week of pregnancy. In some embodiments, the pregnant human is tested before the twenty-sixth week of pregnancy. In some embodiments, the pregnant human is tested before the twenty-eighth week of pregnancy. In some embodiments, the pregnant human is tested before the thirtieth week of pregnancy. In some embodiments, the pregnant human is tested before the thirty-second week of pregnancy. In some embodiments, the pregnant human is tested before the thirty-fourth week of pregnancy. In some embodiments, the pregnant human is tested before the thirty-sixth week of pregnancy. In some embodiments, the pregnant human is tested before the thirty-eighth week of pregnancy. In some embodiments, the pregnant human is tested before the fortieth week of pregnancy. In an embodiment of the method, the pregnant human is tested by week eight of the pregnancy.

Suitable whole blood samples can be in liquid form to allow interaction with the sample well of the device. In certain embodiments, only approximately twenty to thirty microliters (˜20-30 μL) is added to the sample well. In certain embodiments, approximately twenty microliters (˜20 μL) is added to the sample well. In certain embodiments, approximately twenty-five microliters (˜25 μL) is added to the sample well. In certain embodiments, approximately 30 microliters (˜30 μL) is added to the sample well. As only approximately twenty to thirty microliters (˜20-30 μL) is required for the methods as disclosed herein, a simple finger stick can provide more than enough whole blood, obviating the need for venipuncture (since the whole blood can be obtained, for example, by finger stick) and sample processing infrastructure (required to separate serum from whole blood), making the methods and kits as disclosed herein an extremely efficient, low-cost, point-of-care (POC) test for Toxoplasma infection. Lancets can be used in combination with capillary tubes to collect and dispense the whole blood sample. Lancets of varying depths can be used, depending upon the desired blood flow and patient age and weight. When the tip of a capillary tube touches a whole blood droplet drawn by a lancet, blood will flow into the capillary tube via capillary action. After whole blood collection, pressure can be applied to the puncture site with a dry sterile swab until bleeding stops. If an infant's heel was punctured, then the foot should be elevated above the body. Once clotting has occurred an adhesive bandage can be applied if desired.

In certain embodiments, a nitrocellulose strip is used in the lateral flow immunochromatography device. Nitrocellulose membrane layers for lateral flow assays are well-known in the art and are composed of interconnected nitrocellulose fibers. There are multiple benefits to using nitrocellulose for the primary membrane: low cost, capillary flow high affinity for protein biding, and ease of handling. Nitrocellulose has high protein binding. In other embodiments, any substrate capable of providing liquid flow for lateral flow membranes can be used. Such materials are known in the art and include nylon, cellulose acetate, glass fibers, cross-linked dextran and other porous polymers.

In certain embodiments, the nitrocellulose strip comprises a test band (T-band) or test line (T line) and a control band (C-band) or control line (C line). In an embodiment, the test line comprises Toxoplasma antigens. Thus, if anti-Toxoplasma antibodies are present in the whole blood sample from the subject, then the anti-Toxoplasma antibodies in the whole blood sample will bind the Toxoplasma antigens coupled to the test latex particles and to the Toxoplasma antigens in the test line and the test line (“T”) will appear in black. The control line (“C”) will appear regardless of the presence of anti-Toxoplasma antibodies in the sample. The absence of this control line demonstrates the failure of the test and results can't be interpreted.

As used herein, the term “conjugate pad” refers to an area the whole blood first moves through after the sample well and comprises a moveable conjugate of a detectable marker (i.e. a black latex particle conjugated to a Toxoplasma antigen). In certain embodiments, the conjugate pad is a fiberglass pad. When the whole blood to be analyzed flows from the sample well through the conjugate pad, the conjugate (test latex particle coupled to a Toxoplasma antigen) binds to the anti-Toxoplasma antibody present in the whole blood sample, and the complex flows with the whole blood sample to the nitrocellulose strip. Thus, in certain embodiments, the device comprises a conjugate pad in fluid communication with the nitrocellulose strip. In the nitrocellulose strip, the ligand-conjugate complex binds to immobilized binding agent (i.e. Toxoplasma antigen). In some embodiments, the conjugate pad comprises test latex particles coupled with a Toxoplasma antigen. In certain embodiments, the test latex particles are black. In some embodiments, the conjugate pad comprises control latex particles coupled with control antigens. In certain embodiments, the control latex particles are blue.

As used herein, the terms “sample well” or “sample pad” refer to the area of the device where the whole blood sample and the eluting buffer are first dispensed on the device. In certain embodiments, the sample pad of the fluid cassette is in fluid contact with the conjugate pad of the cassette. In some embodiments, the sample pad is a cellulose pad.

Any suitable control can be used, including but not limited to a reference value obtained from one or more subjects that either do not have a T. gondii infection, or that are known to have a T. gondii infection, a previous blood sample obtained from the same subject, or any other suitable control. It is well within the level of those of skill in the art to determine an appropriate control for an intended use in light of the teachings herein. The change in level from control that correlates with T. gondii infection in the subject may be a difference of 10%, 25%, 50%, 75%, 100%, or more. In an embodiment, the difference is a statistically significant increase as judged by standard statistical analysis.

In certain embodiments, the method has a sensitivity of at least about 90%, at least about 91%, at least about 92%, at least about 93%, at least about 94%, at least about 95%, at least about 96%, at least about 97%, at least about 98%, or at least about 99%.

In certain embodiments, the method has a specificity of at least about 90%, at least about 91%, at least about 92%, at least about 93%, at least about 94%, at least about 95%, at least about 96%, at least about 97%, at least about 98%, at least about 99% or at least about 100%.

In certain embodiments, the method further comprises treating those subjects that are identified as likely to have a Toxoplasma infection with a therapeutic for treating Toxoplasma infection. As used herein, the terms “treat,” “treatment,” or “treating” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness. In some embodiments of the method, the subject has toxoplasmosis, and the treating comprises reducing severity of one or more symptoms of toxoplasmosis, and/or reducing recurrence of symptoms of toxoplasmosis. In some embodiments of the method, the treating comprises reducing parasitic load in the subject. In some embodiments of the method, the treating comprises reducing the bradyzoite form and/or the tachyzoite form of the parasite in the subject.

In some embodiments of the method, the method further comprises administering to the subject one or more additional compounds in an amount effective to treat the infection. In some embodiments of the method, the one or more additional compounds are selected from the group consisting of pyrimethamine, sulfadiazine, cycloguanil, inhibitors of calcium kinases or dense granules or vacuolar ATPases, atovoquone, and bulky cytochrome Qi inhibitors, itraconazole and other inhibitors of T. gondii. Therapies may include, but are not limited to pyrimethamine, sulfadiazine, sulfapyrizine, sulfamethazine, sulfamerizine, azithromycin, clarithromycin, atovaquone, dapsone, cotrimoxazole, and combinations thereof (also see WO2017/112678, which is incorporated by reference herein in its entirety). In a further embodiment, the methods may comprise modifying a course of treatment if the subject is not responding appropriately, as determined by attending medical personnel.

In another aspect, the disclosure provides a kit comprising the lateral flow immunochromatography device as disclosed herein. The assembly of the kit can be packaged for use at a single facility where individual components can be grouped to make its use efficient, self-contained and disposable as self-contained for blood borne pathogens or for single use by patient. In the later, in a single small container the following disposable materials are placed: non-breakable capillary tube, finger prick device (e.g. lancet), alcohol wipes, timer for 20-30 minute time alert for reading, link to educational materials, and instructions for use.

In an embodiment, a kit can comprise components for 60 ready to use tests. In such an embodiment, the kit comprises 6 bags (sealed and with zip closure) of 10 devices including a desiccant packet, 3×3 ml of the eluent (dropper bottle), and instructions for use.

Cassettes can be packed in sealed aluminum, PE, or PET bags with a silica gel bag (desiccant). The use of such desiccants is intended to avoid humidity and condensation, due to the very low air permeability of the sealed bag and the silica gel that can capture any residual humidity. The eluting solution is stored in plastic dropper. All batches of dropper are controlled before use to ensure that they do not present a risk of leakage.

In some embodiments, the kit can comprise additional materials, for example, such as a micropipette and disposable tips for dispensing volumes of 15 to 45 μL, and a timer. In certain embodiments, additional materials can include capillary tubes and lancets for fingertip whole blood sampling. In yet additional embodiments, the kits can comprise supplies for general safety precautions, such as gloves and appropriate apparel for protection against exposure to blood borne pathogens. Dry sterile gauze and/or adhesive bandages can also be included in the kits as disclosed herein.

Each kit should be stored in the original sealed bag between 2 and 8° C. (do not freeze). Cassettes can be used until the expiry date written on the bag label. Kits and enclosed devices should not be used after the expiration date. Bags comprising the tests should be allowed to reach room temperature before opening in order to avoid condensation in the bag (e.g. allow at least 15 minutes for the bag to reach room temperature). Keep the bag, after opening at room temperature (18-30° C.), and carefully close (zip closure), with the desiccant packet inside. After opening, the cassettes can be used for up to 2 months. The eluting solution (eluent) is stable up to 2 months at room temperature (18-30° C.) and until expiration date (as written on the kit) if kept between 2 and 8° C.

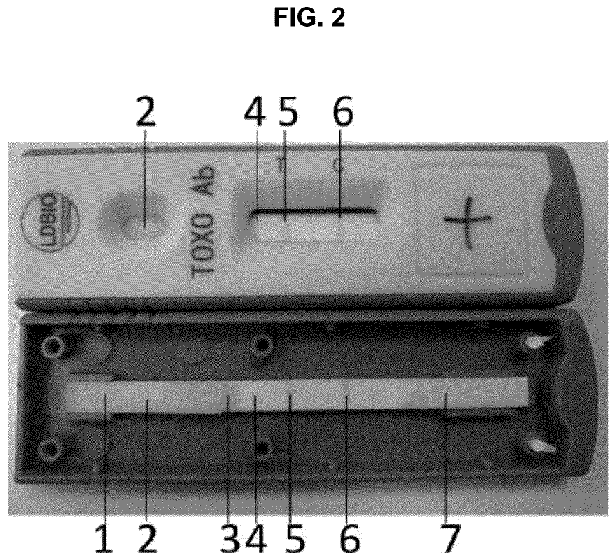

In yet another aspect, the disclosure provides a device. In certain embodiments, the device can be a lateral flow immunochromatographic test strip included in a cassette, which can be composed of a succession of pads allowing a continuous migration of a liquid from one end to the other (chromatography), as shown in FIG. 2. Inside the cassette, the device is composed of: a nitrocellulose strip on which are spread two reactive bands: the antigens (Toxoplasma gondii) of the “test” band (T band) and the rabbit gamma globulins of the “control” band (C band), and a fiberglass support (conjugate pad) which is impregnated of black latex particles coupled with Toxoplasma gondii antigens (“test” latex=T latex) and blue latex particles coupled with goat anti-rabbit IgG (“control” latex=C latex). The test is run by successively dispensing the whole blood sample and an eluting solution (called the eluent) in the sample well of the cassette. The liquid eluent solution, can be composed of solvents (<5% polar ionic solvents) and detergents (<1%), and ensures the chromatographic migration of the sample. Its composition is designed to optimize sensitivity and specificity of the test. Adding the eluent starts the concomitant migration (chromatography) of the serum and the latex particles. This migration is completed in about 20-30 minutes. If specific anti-Toxoplasma antibodies (IgG and/or IgM) are present in the whole blood sample, then a complex is formed between the test latex particles and the patient's antibodies which is then captured by the test band, which results in the appearance of a black line at the T band (i.e., the test is positive). The direct capture of the control latex particles by the C band results in the appearance of a blue line, meaning that the chromatography performed well. The appearance of this blue line is systematic and independent of the anti-Toxoplasma antibody status of the patient. Both letters “T” and “C” can be printed on the cassette, materializing the position of the corresponding reading area.

From left to right (see FIG. 2), the strip can be composed of a cellulose pad called “sample pad” (1) where are dispensed the whole blood sample and the eluent in the “sample well” (2). Then there is a fiberglass pad, called the “conjugate pad” (3) loaded with blue latex particles coated with anti-rabbit goat antibodies and black latex particles coated with Toxoplasma antigens (obtained from in vivo culture). After the conjugate pad, there is a nitrocellulose sheet (4) on which are dispensed two lines of reagent, one of Toxoplasma antigens, the “test line” (5) and one of rabbit antibodies the “control line” (6). If anti-Toxoplasma antibodies are present in the whole blood sample, the test line (“T”) will appear in black. The control line (“C”) will appear regardless of the presence of anti-Toxoplasma antibodies in the sample. The absence of this blue control line demonstrates the failure of the test and results can't be interpreted. Finally, a second cellulose pad at the right-end, the “absorbent pad” (7) completes the strip (see FIG. 2).

The binding of black latex particles to the test line is done by the bivalent property of antibodies (or pentavalence of IgM), as the same antigen is used in both the coating of latex test particles and the test line. The binding of blue latex particles to the control line is done by the direct antibody-antigen reaction between the anti-rabbit goat antibodies (that act as antibodies) and the rabbit antibodies (that act as antigens).

Quality control of the test can be monitored and ensured as follows:
Batch certificate of conformity (homogeneity, analytical performances, stability).
Quality insurance at production and control site (ISO 13189:2016 certified)
Presence of a control line for each test. In the absence, the test must be considered invalid.
Daily/Weekly routine control of a positive and negative control samples, on the same batch number as the one used with patients.

### Reading and Interpretation

An exemplary procedure for the methods, and/or kits and devices as disclosed herein is provided here. Administer approximately 20 to about 30 l of whole blood in the sample well, and then dispense 4 drops of the eluent, keeping the dropper vertical while dispensing. Do not use the eluent of another lot number. Close the dropper after use. Start the timer. The reading must be done near a window or under direct light (example: a desk lamp), and avoid shadows on the reading area. The reading must be done between 20 and 30 minutes after starting the timer, and do not take into account the results from readings after 30 minutes. A positive test is 2 lines, a black “T” line and a blue “C” line appear in the corresponding areas. Every “T” line must be considered positive, even of very weak intensity. For very weak lines, make the reading with the eye vertically above the reading area. For a negative test: no black line appears. Only the blue “C” line is visible. For an equivocal test: in very rare cases, a faint, diffuse, grey line can appear on the “T” band. This result should be considered negative, but controlled on another sample or technique. For an invalid test: the “C” line does not appear. Read once again the instructions and repeat the test. This is a qualitative test. Intensity of the black line does not reflect the quantity of anti-Toxoplasma antibodies in the sample. Positivity of the test is proof of the contact of the patient with the infectious agent, but doesn't prejudge the contact date or the clinical status of the patient. For quality control, the blue “C” line allows the validation of the good running of the test. However it is recommended to incorporate from time to time a known weak positive sample in a run. The positivity can be caused by the presence of IgG and/or IgM directed against the infectious agent, the test does not distinguish the type of antibodies present.

### Manufacturing Process

All steps are guided by procedures and forms, and performed by trained technicians, with a bachelor degree in life science or equivalent and trained to the procedure. Forms are designed to ensure traceability of all steps of manufacturing process and batch number and quantity of materials used. Time spent on all incubation steps is also recorded.

The first step of a production is to purify antigen obtained from in vivo culture of Toxoplasma gondii. At the end of purification, antigen concentration is adjusted by 280 OD to a desired level. The purified antigen is then coupled on black latex particles, using a crosslinker.

The coupled latex particles are then distributed on glassfiber papersheet that are then dried then cut in strips 30 cm long. Purified antigen is also dispensed on 30 cm nitrocellulose cards by a dedicated automated dispenser. Finally nitrocellulose cards, glassfiber strips and two cellulose pads (one sample pad and one absorbent pad) are aggregated together then cut in 3.9 mm width strips. Those strips are then put into the plastic frame.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### EXAMPLES

### Methods:

At each monthly obstetrical visit a finger stick sample with about 20 to about 30 microliters of whole blood was collected following cleansing the fingertip with an alcohol wipe and applying pressure so the fingertip was suffused with blood. The 30 microliters of blood, half the height of the capillary tube, was drawn into the capillary tube. The blood in the capillary tube was applied to the sample well in the lateral flow immunochromatography device, and 4 drops of the eluting solution (eluent) was added. This was performed by a nurse who then cleaned the finger again with an alcohol wipe and applied a small bandage. The medial lateral aspect of the fingertip was used as it is generally less calloused. The lateral flow immunochromatography cassette was placed in a small plastic bag with a timer attached and brought to the appointment with the physician with the timer set for 20 minutes. Two movies were provided to illustrate its use as well as the photographs in the manuscripts provided with the kit. The obstetrician or nurse midwife and his or her nurse read the result, photographed it with a camera and sent it to the central monitor and to the Epic chart in the site at The University of Chicago. Two independent readers documented their observations and there was a photographic record. This can also be performed by heel stick for an infant or with whole blood obtained by phlebotomy in certain circumstances where rapid diagnosis was important. Any such test would always have standard of care test backup so this study would be exempt.

Back up testing was with serum by using either Sabin Feldman dye test/IgM ELISA or Abbott Architect IgG and IgM performed in USA and French reference laboratories. There was 100% concordance when it was tested in the USA in research studies. It was introduced into the clinical setting where it also performed extremely well. The POC test as described herein, has met all the WHO criteria for an ideal POC test including high performance, sensitive, specific, easy to use, rapid, low technology, and inexpensive. It outperformed two other POC tests one made in the US, and one made in Ireland. The lateral flow immunochromatography device also detects false positive IgM results in standard tests used commercially in the US.

### Volunteer Recruitment and Obtaining Samples

Samples were obtained from consenting volunteers, in the U.S. including seropositive individuals affiliated with the NCCCTS and obstetrical patients in Chicago, and obstetrical patients in Morocco (Table 1A-1C). No incentives were provided for participation. Each person of unknown serologic status underwent venipuncture, and status was confirmed either with ARCHITECT Toxo-IgG and IgM system in the Lyon, France Reference Laboratory (n=95 persons), or for Moroccan patients, with Platelia™ Toxo IgG and IgM system (n=39).

TABLE 1A
Methods, Test Results, and Parameters for Sensitivity
and Specificity and Confidence Intervals

| | | | Serology | Serum & | United | |
|---|---|---|---|---|---|---|
| | | | Known | POC Tested | States | Morocco |
| | Patients | Samples | Already | Concurrently | Patients | Patients |
| Seronegative * | 105 | 143** | 8 | 135 | 99 | 6 |
| (IgG and IgM | | | | | | |
| negative in | | | | | | |
| Reference tests) | | | | | | |
| Seropositive * | 100 | 101*** | 64 | 37 | 67 | 33 |
| (IgG and/or IgM | | | | | | |
| positive in | | | | | | |
| Reference tests) | | | | | | |

TABLE 1B
Totals by Country

| | Seronegative | Seropositive | Acute Samples |
|---|---|---|---|
| | Samples Tested | Samples Tested | (IgG + IgM |
| | (by POC and | (by POC and | Tested with |
| Country | Reference Test) | Reference Test) | Reference Test) |
| United States | 137 | 68 | 3^{#} |
| Morocco | 6 | 33 | 2^{#} |
| Total | 143 | 101 | 5^{#} |

TABLE 1C
Test Results, Sensitivity, Specificity, Confidence Intervals

| | Seropositive | Seronegative |
|---|---|---|
| Test Result | (Reference Test) | (Reference Test) |
| Seropositive | 101⁺ | 0 |
| (Whole Blood- POC Test) | | |
| Seronegative | 0⁺⁺ | 143 |
| (Whole Blood- POC Test) | | |

| Test | Diagnostic | 95% Confidence |
|---|---|---|
| Parameter | Performance | Interval |
| Sensitivity | 100% | 96.41-100%⁺⁺ |
| Specificity | 100% | 97.45-100%⁺⁺ |

* For sera samples, pink-line and black-line POC tests were 100% concordant. For whole blood samples, it was determined that the pink-line test kit was not useful when testing whole blood, as the lighter indicator was obscured by the whole blood
** 19 pregnant women were tested a total of 3 times each in an ongoing pilot program for screening during gestation
*** 1 person was tested twice to confirm accuracy of test across and at differing times
^{#}Also included in seropositive person totals. The U.S. persons with IgM antibodies were sometimes followed over time, and all had adjunctive testing such as anti-T. gondii IgA, differential agglutination and avidity tests [2].
⁺Confirmatory tests allow a provider to distinguish acute from chronic infection. Acute infection during gestation requires anti-Toxoplasma medicines to prevent or reduce vertical transmission. Chronic infection requires no further testing during gestation. The first test should be performed by 12 weeks gestation to facilitate distinction of acute and chronic infection [1].
⁺⁺A very faint grey Toxoplasma (T) band was noted transiently for one person tested prospectively. This band was not visible when photographed at 20 and 30 minutes. In accordance with the manufacturer's instructions, this result was designated “equivocal or indeterminate.” Any such equivocal result requires back up testing, as does any first positive result for a pregnant woman.

### Finger Stick Protocol, Whole Blood-POC Test Kit, and Comparison to Serum-Variant Test Kit

Participants provided whole blood via finger stick. Participants' fingers were compressed, suffusing the tip, and cleaned with an alcohol wipe. A standard lancet was used for finger stick. Capillary tubes allowed collection of about 20 to about 30 μL of blood, which was directly applied to the lateral flow immunochromatography device, followed by application of 4 drops of buffer, provided in the kit. Tests were interpreted at 20-30 minutes by the individual performing tests and photographed for later interpretation by two individuals unaware of the subjects' identity and serologic status.

### Ethics

All participants provided written, informed consent. This study was performed in accordance with rules and regulations of the University of Chicago Institutional Review Board under protocol #8793, and/or IRB-approval in Morocco.

### Results:

### Obtaining Samples

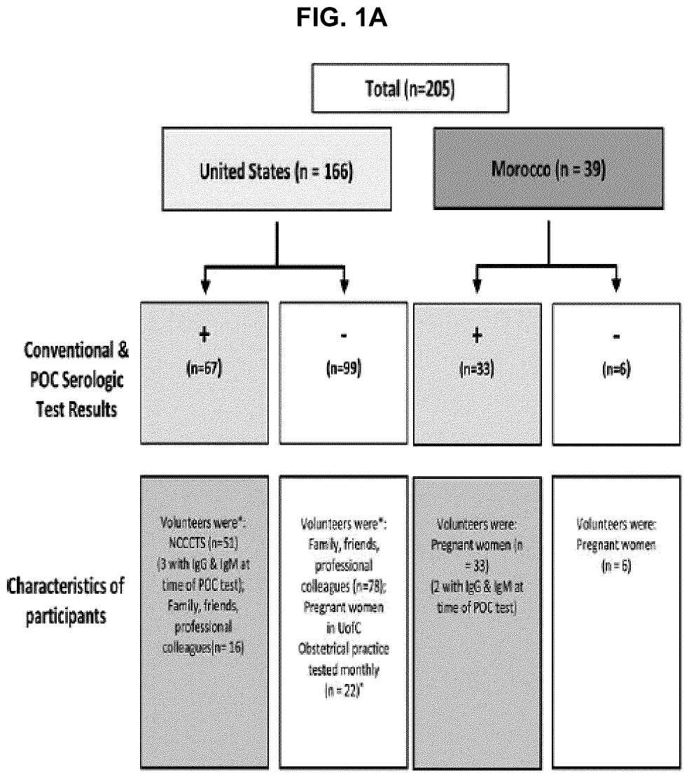

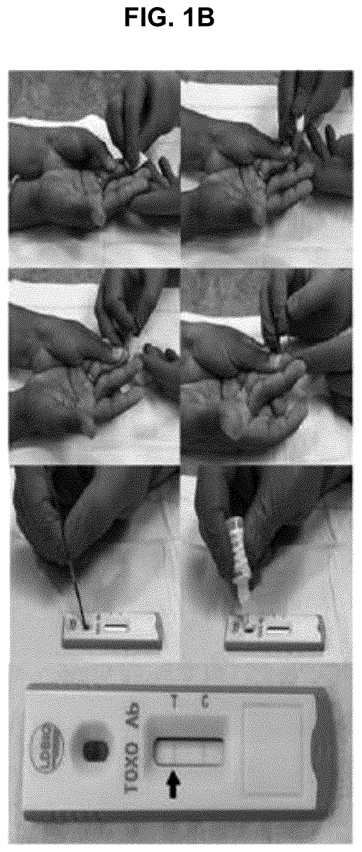

A total of 205 persons (FIG. 1A) (244 samples) had their serologic status for T. gondii assessed using the whole blood test and confirmed using their NCCCTS records (n=71), or concurrent standard laboratory testing (n=134). Nineteen seronegative pregnant women had fingerstick and venipuncture performed each month for the first three months, and three for the first month (two with pregnancy loss) in an ongoing pilot gestational screening program, adding 41 samples, making 244 total samples. Overall, 101 samples proved seropositive, including five from acutely infected individuals, who had IgM/IgG antibodies against T. gondii, while 143 were seronegative. Seropositive is defined here as having detectable anti-Toxoplasma IgG and/or IgM. For a small subset of pediatric patients (n=7, ages 3 weeks to 7 years) who were undergoing venipuncture as part of routine clinical care, whole blood was obtained from the needle tip to avoid a second, potentially distressing fingerstick.

### Sensitivity and Specificity of Whole Blood and Comparability to Serum-Variant Toxoplasma ICT IgG-IgM Test Kit

The whole blood test proved highly sensitive and specific, with sensitivity of 100% (95% CI: 96.41-100%) and specificity of 100% (95% CI: 97.45-100%) (Table 1A-1C). Whole blood, serum-variant, and reference testing demonstrated 100% concordance. Of note, individuals with lower levels of anti-Toxoplasma antibodies infected at remote times and with lower titers were positive in the POC test in the range detected in gold-standard tests.

Viability of Finger stick as Testing Modality

No participant refused a second finger stick, although they were informed they could. Participants did not report any significant discomfort associated with finger stick. Patients and providers enthusiastically accepted the monthly gestational screening program.

### Confirmatory Data

An additional 86 samples were tested for T. gondii using the methods and whole blood-POC test as disclosed herein, and these 86 samples were also assessed using concurrent standard ELISA laboratory testing. Overall, 18 samples proved seropositive, and of the 86 samples tested, there was only 1 discrepancy between the two tests (see Table 2).

The inventors have tested 64 additional people, 43 who were known to be seropositive for T. gondii infection and all were found to be positive in the whole-blood POC test; 21 people were negative in the whole-blood POC test, and were confirmed to be negative by a second standard predicate method (sensitivity 100%, specificity 100%; data not shown).

Additionally, 20 women were tested monthly through 9 months of gestation. The testing consistently showed 100% sensitivity and specificity (data not shown).

TABLE 2
Summary of confirmatory data of whole blood POC test.

| Sample | Whole-blood | |
|---|---|---|
| Number | POC test result | ELISA test result |
| 1 | Negative | Negative |
| 2 | Negative | Negative |
| 3 | Negative | Negative |
| 4 | Negative | Negative |
| 5 | Positive | 70 |
| 6 | Negative | Negative |
| 7 | Negative | Negative |
| 8 | Negative | Negative |
| 9 | Negative | Negative |
| 10 | Negative | Negative |
| 11 | Negative | Negative |
| 12 | Negative | Negative |
| 13 | Negative | Negative |
| 14 | Negative | Negative |
| 15 | Negative | Negative |
| 16 | Negative | Negative |
| 17 | Positive | 60 |
| 18 | Negative | Negative |
| 19 | Negative | Negative |
| 20 | Negative | Negative |
| 21 | Negative | Negative |
| 22 | Negative | Negative |
| 23 | Positive | 48 |
| 24 | Negative | Negative |
| 25 | Negative | Negative |
| 26 | Negative | Negative |
| 27 | Negative | Negative |
| 28 | Positive | 48 |
| 29 | Negative | Negative |
| 30 | Positive | 48 |
| 31 | Negative | Negative |
| 32 | Negative | Negative |
| 33 | Negative | Negative |
| 34 | Positive | Negative |
| 35 | Positive | >240 |
| 36 | Negative | Negative |
| 37 | Negative | Negative |
| 38 | Positive | 60 |
| 39 | Negative | Negative |
| 40 | Negative | Negative |
| 41 | Negative | Negative |
| 42 | Negative | Negative |
| 43 | Negative | Negative |
| 44 | Negative | Negative |
| 45 | Negative | Negative |
| 46 | Positive | * |
| 47 | Positive | * |
| 48 | Negative | Negative |
| 49 | Negative | Negative |
| 50 | Negative | Negative |
| 51 | Negative | Negative |
| 52 | Negative | Negative |
| 53 | Positive | 82 |
| 54 | Positive | 90 |
| 55 | Negative | Negative |
| 56 | Positive | 240 |
| 57 | Positive | 194 |
| 58 | Negative | Negative |
| 59 | Negative | Negative |
| 60 | Negative | Negative |
| 61 | Negative | Negative |
| 62 | Negative | Negative |
| 63 | Negative | Negative |
| 64 | Negative | Negative |
| 65 | Negative | Negative |
| 66 | Negative | Negative |
| 67 | Negative | Negative |
| 68 | Negative | Negative |
| 69 | Negative | Negative |
| 70 | Negative | Negative |
| 71 | Negative | Negative |
| 72 | Negative | Negative |
| 73 | Negative | Negative |
| 74 | Negative | Negative |
| 75 | Negative | Negative |
| 76 | Negative | Negative |
| 77 | Positive | >240 |
| 78 | Negative | Negative |
| 79 | Negative | Negative |
| 80 | Negative | Negative |
| 81 | Positive | 158 |
| 82 | Negative | Negative |
| 83 | Negative | Negative |
| 84 | Positive | 194 |
| 85 | Negative | Negative |
| 86 | Positive | 110 |

* confirmed positive with BioRad serology test

### DISCUSSION

Point-of-care testing for Toxoplasma infection has potential to markedly change clinical approach to this infection by detecting seroconversion, using small volumes of whole blood, obviating venipuncture and expensive equipment for serum separation, and performing with high sensitivity and specificity.

The point-of-care testing for Toxoplasma infection using whole-blood as disclosed herein meets all of the WHO criteria for POC testing. It is an Affordable, Sensitive, Specific, User friendly, Rapid/robust, Equipment free, and Deliverable to end users (A.S.S.U.R.E.D.).

Adoption of this whole-blood POC test would reduce costs further substantially and require significantly less infrastructure than conventional testing. Value also arises from bringing pregnant women into care, ensuring screening for other preventable and treatable conditions, particularly in developing countries [6]. This whole blood POC test was not used to distinguish seropositivity for IgG and IgM, and therefore did not distinguish acute from chronic infection. Confirmatory testing and guidance concerning additional testing and treatment will benefit patients and their physicians. In countries with high seroprevalence, this benefit would be particularly important. This POC test demonstrated superb diagnostic performance in countries in the developed and developing world, with genetically distinct patients and parasites.

### Advantages:

True point-of-care test, useful in a standard obstetrician's office and less invasive than conventional serologic testing (requiring materials to perform venipuncture, electricity and a centrifuge for serum separation, and skilled technicians). POC testing has potential to significantly expand access to screening during gestation for this serious, potentially fatal infection, with spillover benefit in facilitating screening programs for other congenital infections and improvements in maternal fetal health and well-being and care. The high performance of this whole-blood POC test as demonstrated herein, and the test's strong functionality at the point-of-care, which has not been previously demonstrated, provides the proof of principle of its potential utility and widespread applicability in clinical settings. This new test also fulfills the World Health Organization criteria for the ideal POC test (Affordable, Sensitive, Specific, User-friendly, Rapid/robust, Equipment-free, and Deliverable to users).
Performance and interpretation of test takes less than two minutes of operator time, with results available to be interpreted within 20-30 minutes, facilitating appropriate clinical intervention so that it is ideal for following those who are seronegative to detect seroconversion.
There is no need for expensive equipment/electricity, decreasing cost and enhancing utility, especially in developing countries.

### REFERENCES

1. McLeod R, Wheeler K, Levigne P, Boyer K. Toxoplasma gondii. In: Read J S, Schleiss M R, eds. Congenital and Perinatal Infections. Oxford University Press, 2018: 223-239.
2. McLeod R, Lykins J, Noble A G, et al. Management of Congenital Toxoplasmosis. Curr Pediatr Rep 2014; 2:166-194.
3. Stillwaggon E, Carrier C S, Sautter M, McLeod R. Maternal Serologic Screening to Prevent Congenital Toxoplasmosis: A Decision-Analytic Economic Model. PLoS Negl Trop Dis 2011; 5:e1333.
4. McLeod R, Boyer K, Karrison T, et al. Outcome of Treatment for Congenital Toxoplasmosis, 1981-2004: The National Collaborative Chicago-Based, Congenital Toxoplasmosis Study. Clin Infect Dis 2006; 42:1383-1394.
5. Wallon M, Peyron F, Cornu C, et al. Congenital Toxoplasma Infection: Monthly Prenatal Screening Decreases Transmission Rate and Improves Clinical Outcome at Age 3 Years. Clin Infect Dis 2013; 56:1223-1231.
6. Begeman I J, Lykins J, Zhou Y, et al. Point-of-care testing for Toxoplasma gondii IgG/IgM using Toxoplasma ICT IgG-IgM test with sera from the United States and implications for developing countries. PLoS Negl Trop Dis 2017; 11:e0005670.
7. Chapey E, Wallon M, Peyron F. Evaluation of the LDBIO point of care test for the combined detection of toxoplasmic IgG and IgM. Clin Chim Acta 2017; 464:200-201.
8. Maldonado Y A, Read J S, Diseases C on I. Diagnosis, Treatment, and Prevention of Congenital Toxoplasmosis in the United States. Pediatrics 2017; :e20163860.
9. Prusa A-R, Kasper D C, Sawers L, Walter E, Hayde M, Stillwaggon E. Congenital toxoplasmosis in Austria: Prenatal screening for prevention is cost-saving. PLoS Negl Trop Dis 2017; 11:e0005648.
10. Mahinc C, Flori P, Delaunay E, et al. Evaluation of a new ICT test (LDBIO Diagnostics) to detect Toxoplasma IgG and IgM: comparison with the routine Architect technique. J Clin Microbiol 2017; :JCM.01106-17.
11. Toxoplasma Gondii—The Model Apicomplexan—Perspectives and Methods, 2nd Edition —Aug. 10, 2013, Academic Press, Editor: Louis M. Weiss—Chapter 4.

### Accurate Monthly Gestational Screening to Prevent Congenital Toxoplasmosis and More

We asked whether high performance of an Immunochromatographic-test (hereinafter “ICT”) could enable accurate, rapid diagnosis/treatment, establishing new, improved care-paradigms at point-of-care and clinical laboratory.

Toxoplasmosis is a major health burden for developed and developing countries, causing damage to eyes and brain, loss of life and substantial societal costs. Prompt diagnosis in gestational screening programs enables treatment, thereby relieving suffering, and leading to >14-fold cost savings for care. Herein, we demonstrate that using an ICT that meets WHO REASSURED-criteria identifying persons with/without antibody to Toxoplasma gondii in sera and whole blood with high sensitivity and specificity, is feasible to use in USA clinical practice. We find this new approach can help to obviate the problem of detection of false positive anti-T. gondii IgM results for those without IgG antibodies to T. gondii when this occurs in present, standard of care, predicate USA FDA cleared available assays.

Thus, this accurate ICT facilitates gestational screening programs and a global initiative to diagnose and thereby prevent and treat T. gondii infection. This minimizes likelihood of false positives (IgG and/or IgM) while maintaining maximum sensitivity. When isolated IgM antibodies are detected, it is necessary to confirm and when indicated continue follow up testing in ˜2 weeks to establish seroconversion. Presence of a positive ICT makes it likely that IgM is truly positive and a negative ICT makes it likely that IgM will be a false positive without infection. These results create a new, enthusiastically-accepted, precise paradigm for rapid diagnosis and validation of results with a second-line test. This helps eliminate alarm and anxiety about false-positive results, while expediting needed treatment for true positive results and providing back up distinguishing false positive tests.

A test that fulfills all of the WHO ASSURED (Affordable, Sensitive, Specific, Robust, Equipment free, Deliverable to users) criteria for persons who are infected due to exposure to T. gondii parasites of the many haplogroups in persons of varying genetics in 7 countries on 5 continents and multiple island nations and states including Canada, the USA mainland, Hawaii, Mexico, Panama, Colombia, French Guiana, Guadeloupe, Brazil, Philippines, multiple locations in France, Morocco and New Zealand in more than 6000 persons' samples, in many varied demographics, documented in studies with multiple testers and types of testers, in multiple settings including automobile drive by, home outdoor, physician offices, nurses' offices, employee health, phlebotomy laboratory, research laboratory, clinical laboratory, conference room, and office waiting areas, and other settings, and with line data identifying those persons as immunocompetent or immune compromised having eye or brain disease, being a mother of a congenitally infected child, being pregnant or not.

The test disclosed herein successfully detects seropositivity during seroconversion in acute infection as well as chronic infection and recognizes seronegative persons on multiple continents and countries when compared with comparator settings in persons in their own countries, it is easy to understand its use in multiple settings by testers with multiple skill sets in all these countries the universal novel test that includes documentation of each and any of the above properties and functionalities which will save the lives and well-being of members of hundreds of thousands of families saving large amounts of money as documented by cost benefit analyses for individuals and governments while preventing lifelong morbidity and suffering.

The ICT disclosed herein was compared with gold-standard or predicate-tests. Overall, the ICT performance for 1093 blood/4967 sera was 99.2%/97.5% sensitive and 99.0%/99.7% specific. However, in clinical trial, FDA-cleared-predicate tests initially caused practical, costly problems due to false-positive-IgM results. For 58 persons, 3/43 seronegative and 2/15 chronically infected persons had false positive IgM predicate tests. This caused substantial anxiety, concerns, and required costly, delayed confirmation in reference centers. Absence of false positive ICT results contributes to solutions: Lyon and Paris France and USA Reference laboratories frequently receive sera with erroneously positive local laboratory IgM results impeding patient care. Therefore, thirty-two such sera referred to Lyon's Reference laboratory were ICT tested. We collated these with other earlier/ongoing results: 132 of 137 USA or French persons had false-positive local laboratory IgM results identified correctly as negative by ICT. Five false positive ICT results in Tunisia and Marseille, France, emphasize need to confirm positive ICT results with Sabin-Feldman-Dye-test or western blot. Separate studies demonstrated high performance in detecting acute infections, meeting FDA, CLIA, WHO REASSURED, CE-Mark criteria and patient and physician satisfaction with monthly-gestational-ICT-screening.

### Conclusions Significance

This novel paradigm using the ICT identifies likely false positives or raises suspicion that a result is truly positive, rapidly needing prompt follow up and treatment. Thus, the ICT enables well accepted gestational screening programs that facilitate rapid treatment saving lives, sight, cognition and motor function. This reduces anxiety, delays, work, and cost at point-of-care and clinical laboratories.

This ICT lateral immunochromatography test with antigen on beads that captures IgG and/or IgM antibodies, Toxoplasma ICT IgG-IgM test (LDBIO Diagnostic, Lyon, France, hereafter called ICT) is a promising candidate NRL test that satisfies REASSURED criteria [23-5].

Nonetheless, positive results influencing clinical care still require clinical reference laboratory confirmation.

### Objectives

In 12 sequential studies and analyses we aimed to learn how well the ICT met WHO REASSURED criteria and guidelines for achieving FDA clearance and CLIA waiver in the USA and CE Mark in Europe. This work also was designed to determine whether it is feasible to use the ICT, maintaining its high performance, with users in multiple clinical circumstances and settings. We also intended to determine acceptability of the use of the ICT to patients, families, and front-line health care personnel. In so doing we intended to learn how the ICT also might be used optimally in the clinic and in the laboratory for rapid back up testing to address whether false positive results in standard commercial tests might have occurred. If these criteria were met, this test could help to enable serologic screening to detect, diagnose and thereby promptly treat to prevent this infection and its adverse sequelae providing an improved paradigm for care to prevent or ameliorate the manifestations of this infection. When we encountered difficulties in the first 6 tests with false positive IgM results with the commercial, predicate comparator compared with gold standard tests in our feasibility clinical trial, but not the ICT, we were faced with the unanticipated constraint of cost of positive confirmation of a number of tests at PAMF-TSL, and recognition that this type of cost from the frequent false positive IgM results could de-rail screening programs in the United States. Given the true negative ICT results when compared with gold standard tests in our setting, we queried whether the ICT could be part of a paradigm to rule-out false positive IgM results with NRL test, both at the point-of-care and in the hospital clinical laboratory. Further, we tested samples with the ICT that were suspected false positives from local laboratories that had been referred to reference laboratories.

### Implications of Unexpected Findings Leading to Next Steps

Our unanticipated findings of false positive NRL commercial predicate tests led us to identify a solution for our clinical problem: We placed these data in the context of practical clinical problems we encountered and collated our results with ongoing and reported similar studies to define whether this could be an approach helpful in addressing false positive predicate NRL test results. Solving this problem emphasized how the ICT can be used in screening programs to benefit pregnant women and their families, creating a new model to approach the problem of need for accurate screening and of false positive NRL tests. This highly accurate test may help enable screening for acquisition of T. gondii in gestation [23-39], as well as being useful in other settings, and thereby contribute to saving sight, cognition, motor function and lives and improve quality of life [1-10,12-15,17,24,35].

### Methods

### Ethics

The study was conducted with ethical standards for human experimentation established in the Declaration of Helsinki. Research received approval from the University of Chicago Institutional Review Board (University of Chicago IRB Protocols 20-0442, 19-0505, 21-1446, 8793, 8794, 8795, 8796, 8797, 8798, 16708A and met the standards of the Health Insurance Portability and Accountability Act).

### Overall Goals

These methods were structured to meet our initial specific objectives to determine the performance of the ICT. When we found high performance in initial studies objectives were then to carry out studies to assess whether the test met WHO REASSURED criteria for a point of care test, to meet all CE Mark, FDA and CLIA testing requirements, and to determine feasibility of implementation of use of ICT in multiple “real-life” clinical settings.

### Design

We performed a series of studies with a lateral immunochromatography test (ICT) we previously found met WHO REASSURED criteria with serum and whole blood from a finger prick. This new work performed herein included prospective real time studies of feasibility and acceptability in the USA, France, and Colombia. In so doing, we discovered paradigm shifting approaches and utility, proving our original hypotheses and extending beyond these hypotheses. These studies used methods that follow. Box 1 lists and provides a “roadmap” to and milestones for 12 studies/analyses in this decades-long work. An overview of methods follows:

### Box 1. Roadmap and Milestones Create a Flow Chart of Studies, Analyses, Content, Supporting Figures, Boxes, and Tables

Line data Links in Tables 1, 2

Box 1. Roadmap and Milestones Create a Flow Chart of Studies,
Analyses, Content, Supporting Figures, Boxes, and Tables

| Study No. | Topic | FIGS. | Boxes | Tables |
|---|---|---|---|---|
| 1 | Feasibility Clinical Trial | 1, 2A | 1, 2 | 1, 2, A^{a} |
| 2 | False positives a-Lyon, b-Paris | 2B, 2C | 1, 2 | 1, B^{a} and C^{a} |
| 3 | Acceptability Monthly screening, USA | 4 | 1, 2 | 1, C^{a}, 3 |
| 4 | Pink/Black, All studies analysis, Novel Paradigm | 3 | 1, 2 | 2, 3, 4 |
| 5 | Time/Cost Analysis | | 1 | 4 |
| 6 | Quick Information/Limits of Detection (LOD) [symbol] | 2F, 2G. | 1, 3 | |
| 7 | Acute Infection Colombia, USA | 2E | 1, 2 | 5 |
| 8 | Additional Chronic USA | | 2 | |
| 9 | Epidemiology studies | 5 | 2 | |
| 10 | False positive/negative AD Bio, Colombia, USA | 2D | 1, 2 | 5, 6 |
| 11 | Commentary: Case summaries | | A^{a} | |
| 12 | Commentary: Chronology/Context | A [symbol] , B [symbol] | 1, 2 | 1^{b}, 2^{b}, 6 |
| Summary | Summary of Results on Roadmap | 6 | 1 | |

[symbol]

[symbol]

Links in Tables 1 [symbol] 2 [symbol]
indicates data missing or illegible when filed

S1 commentary is in PLOS Neglected Tropical Diseases, Zhou Y, Leahy K, Grose A, Lykins J, Siddiqui M, Leong N, et al. (2024) Novel paradigm enables accurate monthly gestational screening to prevent congenital toxoplasmosis and more (hereinafter Zhou et al.). PLOS Negl Trop Dis 18(5): e0011335. Hereinafter S1 refers to the supplement section of this citation. The numbers for the figures boxes and tables are from Zhou et al. and in this specification and figures we keep these and have numbered them consecutively starting with FIG. 4 and Table 3 in this specification.

### Study 1: Feasibility Clinical Trial Study

Overall. The design of this Study 1 (Clinical Trials.gov number NCT04474132) and how it is related to earlier work is shown in FIG. 4A-4E. To begin to implement a reasonable, feasible, low cost workflow for USA gestational screening programs, which has currently and historically been problematic, a series of 12 studies described herein were performed. A formal clinical trial feasibility study at the University of Chicago Medical Center (UCMC) began in July 2020. The goal was to evaluate a sufficient number of verifiable ICT results to complete the FDA (510(k) clearance and CLIA regulations waiver process. This study involves comparing results of the ICT to an already cleared serum test, also called predicate test (Bio-Rad Bioplex 2200 Toxo Enzyme Immunoassay). Serologic samples for the UCMC feasibility study 1 (ongoing as of February 2024) were obtained from 41 pregnant women, 40 undergoing regular prenatal appointments at the UCMC (23 in first trimester, 12 in second trimester, four in third-trimester) and from seventeen non-pregnant volunteers. Each participant's whole blood and sera were tested with ICT; Participants' sera were also tested at the UCMC's CLIA-approved Clinical Laboratory, which uses an automated Bio-Rad Bioplex 2200 Toxo Enzyme Immunoassay as its FDA cleared standard predicate test to detect IgG and IgM anti-Toxoplasma antibodies. For this ICT feasibility clinical trial, there were three testers in 3 different sites in the University. All discrepant results between ICT and predicate were sent to Remington Specialty Laboratory-PAMF-TSF or Lyon Reference laboratory for confirmation immediately using a panel of tests described elsewhere [27,37]. Specifically, as the FDA guidelines specified, this was carried out to determine whether this new ICT could be used successfully within formal, usually structured, clinical care systems in a University outpatient obstetrical and in-field practice settings. It was compared with the simultaneously performed FDA cleared, standard, predicate test. The ICT with whole blood was read by three readers independently and with sera by two independent readers also at the same times. Three different healthcare providers performed the point of care (POC) test. Each provider tested a minimum of five sero-positive persons. Results were entered into the University hospital EPIC medical record, standard clinical laboratory, and Clinical Research Redcap systems of the University of Chicago Hospitals with ICT results also documented by smart phone photograph.

This was tested to determine whether this novel, simple test could perform well even during a time with the constraints posed by the SARS CoV-2 pandemic. As the FDA clearance, CLIA waiver and Institutional Review Board (IRB) processes specified must occur, this was a formal Clinical Trial included in Clinical Trials.gov NCT04474132, that would mimic ways the test would be used to identify serologic positivity or seroconversion during pregnancy in the University of Chicago Medicine practice or in other clinical settings that would be suitable to apply for dual FDA clearance and CLIA waiver. This was to formally determine whether this test could perform well and be feasible and easy to use within a clinical trial in a standard obstetrical and other care and research settings in accordance with FDA and CLIA requirements. We were expected to use a standard, comparator, “predicate” test that the FDA had already cleared for use with serum in the USA to detect and confirm presence of antibodies to T. gondii. There were only three such cleared tests in the USA and we selected the test our clinical laboratory utilized regularly, herein designated “predicate comparator test” so it could exactly replicate the academic practice setting in accordance with FDA/CLIA guidelines. The test cost was lowered for the clinical trial from $650 for the IgG and IgM clinical laboratory test to $13 to facilitate the study. The cost of the predicate testing was paid for by the Seed Fund Award from the Susan and Richard Kiphart Family Foundation, the A.K. Thrasher Children's Charity and other charitable sources of funds. US Reference laboratory confirmation of test results could not be subsidized and remained over $1600 for two consecutive tests with a full “adult panel” when a positive IgM test was identified. Back up testing was performed for two patients in the Remington Toxoplasma Specialty Reference Laboratory and also was performed in the Lyon reference laboratory without charge using Abbott ELISA IgM/IgG. In August 2022 the Abbott IgG reagents also became FDA cleared.

### Whole Blood Sample Testing Protocol for ICT.

For each ICT, about 20 to about 30 μL of whole blood was collected using a finger prick and a 60 μl glass micro hematocrit tube filled to half full (by visual estimate) and placed into the well of the device. After placing four drops of diluent buffer in the well and waiting for 20 to 30 minutes, each ICT test yielded results that were interpreted by the tester and two additional readers independently reading photographs of the results. All individuals who interpreted results were blinded to the Bio-Rad IgG and IgM test results. Test interpreters determined whether the ICT result was positive (black, positive line and blue, positive control line) or negative (absence of the black, positive line and presence of blue, positive control line). According to the test Instructions for Users (IFUs) its performances are 97.5% sensitivity and 99.7% specificity when used with whole blood.

### Predicate Test Protocol.

Toxoplasma IgG or IgM (Bio-Rad) (Bioplex 2200 automated assay) detects IgG or IgM antibodies against T. gondii via capture of IgM in solid phase. Microplate wells coated with anti-human antibody chains, a mixture of antigens and monoclonal anti-T. gondii antibody labeled with peroxidase, the conjugate, are used. Values ranging from 0.8 to 1 were “equivocal”, values >1 were considered positive for IgM. These tests were used in accordance with the manufacturer's instructions. When the first false positive IgM tests results were detected Bio-Rad validated proper functioning of the test materials and automated machine at UCMC and re-calibrated and re-standardized the machine. This did not eliminate the false positive results which seemed to be associated with the reagents and system intrinsically. Confirmation of discrepant results for Clinical Trial. All discrepant results between ICT and predicate were sent to Remington Specialty Laboratory- PAMF-TSF or Lyon Reference laboratory for confirmation immediately using a panel of tests described elsewhere [27,37].

Study 2a: Samples from Lyon Reference Laboratory that had been referred when reported/referred by local laboratories with erroneous positive IgM A set of 32 samples obtained at the Parasitology Laboratory of the University Hospital of Lyon, France (Institut des agents infectieux, Hospital de la Croix-Rousse, Lyon, France) were selected for being reported as false positive with at least one first-line, NRL automated assay and confirmed to be negative by a panel of additional tests in the laboratory including Abbott Architect G, M, Vidas G and Bio-Rad Platelia IgM ELISAs. They were in some cases additionally tested at LDBIO Diagnostics using ICT and WB ToxoII IgG which is a gold standard in France [40-45].

Study 2b. Testing of Other Samples at Hospital Bichat, Paris

A total of 558 US serum samples that had been tested in a one week time period at the University of Chicago Medicine Clinical Laboratory, then stored for one week, that would otherwise be discarded were tested at Ho\hat{ }Pital Bichat, Paris (Bichat-Claude Bernard Hospital, Laboratory of Parasitologie, Paris, France [30]). Another set of French samples also was tested at Hospital Bichat in Paris. Results are being presented in detail in a separate report describing a variety of tests from Ho\hat{ }Pital Bichat [30]. Twenty-two false positive NRL IgG and 6 false positive NRL IgM samples were tested in this part of the study. Follow-up without seroconversion and other reference tests were used to confirm that positive results were false positives.

### Study 3: Testing of US Samples in a Study to Determine Feasibility and Acceptability of Finger Stick in Monthly US Gestational Screening Program 2017-2022

Practice setting and patient recruitment. This separate study was to determine whether this ICT testing could be performed monthly for pregnant women in an academic obstetrical setting in the USA and whether it would be acceptable for patients and their physicians. This study took place in the outpatient Obstetrics and Gynecology Practice at an urban academic medical center between September, 2017 and September 2018. Patients were identified at their first outpatient obstetrical visit, between 8-12 weeks gestation, by their primary obstetrical care provider.

Patients not infrequently attended their obstetrical visit with their partners. They were provided an educational pamphlet [33] and were able to ask any questions. Patients then were offered an opportunity to participate in the monthly screening study and if they wished to do so to sign an informed consent. If the patient indicated interest in participating in the study, voluntary consent was obtained by the research team. All patients who were asked expressed interest and willingness to participate. Original intent of the study was to follow 20 women to term with monthly testing through the sixth week post-partum obstetrical visit.

Testing. Each month, at the patients' regularly scheduled appointment or shortly thereafter the patient was tested with the whole blood-variant Toxoplasma ICT IgG-IgM POC test. Methods for testing have been discussed in our previous work [23] and above. Serum was tested with another high-functioning test, i.e., with the ARCHITECT-(Abbott, United States), and/or as an automated enzyme-linked fluorescent immunoassay (ELFA, (VIDAS Immuno-Diagnostic Assay System, Biomerieux, France)) and/or Western Blot-Toxo-IgG and IgM systems (LDBIO diagnostics) performed in Lyon, France and/or Quindio Colombia Reference Laboratories [10,25]. One hundred fifty-five total tests of each type, i.e., ICT on serum and blood and reference lab G and Mtests in Lyon were performed. Eighty-eight of these were also tested with Vidas IgG and IgM in Quindio. We also tested an additional 25 participants in the National Collaborative Congenital Toxoplasmosis Study (NCCCTS) and our other studies during this time frame who wanted to participate.

Provider participation and patient satisfaction surveys. Providers joined the study as collaborators following an Obstetrics Department Grand Rounds and Obstetrics Sectional Educational informational meeting for those who missed the Grand Rounds. Both informational meetings were presented by RMc. Providers were given the same educational pamphlet that their patients also received. All had the opportunity to ask questions of RMc and learned about various related educational resources [1-3,6,7,32-35,38,46]. As described above under “Patient Recruitment”, providers then mentioned the study to their patients. At the initial and subsequent visits the medical student (JL), Maternal Fetal Medicine Nurse (KL), or PI (RMc) obtained the samples after coordinating with the patient and practitioner at the time of a subsequent monthly obstetrical visit. Providers were told the results they could discuss with their patients. Surveys, designed to assess patient satisfaction with the gestational screening program were created to use at the end of the study. Responses were based on a 5-point Likert scale, ranging from “strongly disagree” to “strongly agree.” There was also an opportunity for free response regarding strengths and potential areas of improvement for the screening program. The details of the questions are in the results section. Surveys were provided by the research nurse or others working in the study to the study participants at the 6-week postpartum visit or shortly before this visit. Contact with provision of the brief questionnaire was missed for five study participants at the 6-week postpartum visit. All five were asked and two of those five completed the questionnaire at a later time. Surveys were anonymized, so correlation of survey data to individual respondents was not possible. As part of this intent-to-study, as above, we had enrolled 22 pregnant women, and 19 continued monthly. In the Lyon, France reference laboratory, the 155 samples were tested with Abbott ELISA IgG/IgM. When Abbott Architect (France) IgG/IgM had either an IgG or IgM that was positive, backup testing was performed with VIDAS in Lyon laboratory, and LDBIO Western Blot IgG/IgM (IgM performed for three tests at LDBio). In the Quindi\acute{ }o, Colombia reference laboratory that uses VIDAS family (Immuno Diagnostic Assay System) as an automated enzyme-linked fluorescent immunoassay (ELFA) test, the last 88 of the 155 samples were tested in parallel. Additionally, results of evaluations presented in Medical/Scientific congress that were known by the authors including those in submission to another journal was added to the totals in this analysis.

Study and Analysis 4. Comparison of pink bead used earlier and black bead used in later studies and herein (Study 4) and Collation of Earlier Testing, Bibliographical search, and development of novel paradigm (Analysis 4) Study 4. A comparison of the performance of the pink line/red bead test kits used in early studies and black bead test kits were performed to determine that the performance of the test kits using these different beads were comparable: 1074 serum sample were tested with the pink line/red bead and black line test kits and Abbott Architect in Lyon (N =200; Wallon), Marseille (N=200; L'Ollivier), Ste Etienne (N=374; Mahinc, Flori), LDBIO, (N=300; RP). Any discrepancies were resolved with Western blot. This is part of the formal CE Mark, French Reference Laboratories, and FDA/CLIA file submissions of line data that also were reviewed in Chicago.

Analysis 4. We collated results of all of our earlier work, both published already [23-27,29-31,35] and other separate studies ongoing at present on related topics that were published separately concurrently [30], and our current work herein. Our earlier work and some of the work presented herein was used to apply for European CE mark which was granted in October 2020. Now that the ICT is CE marked and available in Europe, to determine whether we had overlooked any other study we were not otherwise aware of, we performed a bibliographical search on Pubmed using terms for evaluations of the Toxoplasma ICT IgG-IgM test. This was to make certain that we had included all reported tests in our analysis. Only English literature was reviewed. For evaluations found, full text was retrieved and searched for potential false positive samples. Additionally, results of evaluations presented in congress that were known by the authors including those in submission to other journals were added to the totals in this analysis. Earlier and ongoing studies and reports were arranged chronologically and the total collated data are reported herein.

Summary diagram of novel algorithm the work presented herein develops. Difficulties we encountered initially in our clinical trial inspired organizing the algorithm we created to prevent problems like those we had to address.

Study/analysis 5: Time cost analysis. We found a number of approaches including reference laboratory tests with varying costs, ease of use, and considered whether they meet WHO REASSURED criteria. Advantages and disadvantages of those approaches are summarized in tabular format including a time cost analysis.

Study 6: Evaluation of instructional materials for ICT with whole blood at point of care in limit of detection/quality of instructions (LOD/QI) study in accordance with FDA/CLIA guidelines. The following study was performed to determine the precision of the ICT with samples at the limit of detection and whether never experienced testers could read, understand, perform and interpret instructional material for use of the ICT with whole blood.

### Sample Preparation

Samples were prepared in accordance with FDA/CLIA requirements and guidance for instructional material for CLIA waiver for a point of care test (Recommendations for Clinical Laboratory Improvement Amendments of 1988 [CLIA] Waiver Applications for Manufacturers of In Vitro Diagnostic Devices ([version of Jan. 30, 2008-in force and updated in 2020]). A serum sample that had T. gondii antibody, that was positive at the limits of detection for the Toxoplasma ICT IgG-IgM test was used as positive sample. The sample was prepared according to CLSI EP12-A2 User Protocol for Evaluation of Qualitative Test Performance guideline, with the objective of obtaining a sample that would be positive around 95% of the time (defined as positive between 36 to 39 times out of 40 trials, as per guideline instruction). The sample used was a citrate-based plasma obtained from the Etablissement Frangais du Sang (the French national blood bank) with both IgG and IgM for Toxoplasmosis (38 UI/ml of IgG and 63.89 ratio for IgM according to Roche Toxoplasma kits) prior dilution into seronegative heparinized blood obtained from venipuncture of a known seronegative person the day prior to dilution. The selected dilution was the 90th (1:89 ratio) as the test was read positive 37 times by an untrained user and 39 by an experienced user at that dilution (LDBIO Diagnostics). Assuming linearity of dosage for the Roche kit, the IgG titer of the final sample was therefore 0.42 UI/ml, and 0.71 ratio for IgM, both below the threshold of 3 and 1 for the Roche IgG and IgM kit, respectively. Knowing that this limit of detection was determined to occur when this positive serum was diluted 1:89 with whole blood, serum or plasma from a seronegative donor, the study proceeded as follows: Prior to being tested by nine testers, the whole blood from another seronegative donor in Chicago was confirmed to be negative with ICT from finger stick. Thirty ml of whole blood was drawn from this second seronegative donor by venipuncture and placed into three ten ml tubes with EDTA anticoagulant and gently mixed by repeatedly inverting the tubes. This blood was divided into two parts, one part was to be used to create the negative samples and the second part was to be used to create the “limits of detection positive” sample. Half the blood from the seronegative donor was diluted 89:1 with the previously tested positive serum and gently mixed to create the “positive at the limits of detection whole blood sample”. The ability of an experienced tester to distinguish the negative and positive samples was tested with the ICT and was confirmed. Two hundred microliters of the negative whole blood was placed into each of sixty-three 1.5 ml Eppendorf tubes used in this study for the negative samples. Two hundred microliters of the “positive at the limits of detection whole blood sample” was placed in each of sixty-three 1.5 ml Eppendorf tubes. The samples were assigned and labeled with random numbers between 1 and 14. The code was known only to the scientist who diluted the samples and prepared the labeled tubes and created the unknowns to be tested. A set of unknown blood samples labeled from #1 to #14 was prepared for each of the nine testers along with ICT cassettes labeled with the number between 1 and 14. Each set had the testers initials on each cassette.

Each tester had the same samples labeled 1 to 14. The limits of detection, “Quick Instructions” (“QI”) study was then performed as follows: Nine testers were identified: Testers: The testers were asked to read and sign the informed consent if they concurred. They were asked to read the information (Quick information, QI) document and complete the top of the data sheet documenting they had read the QI and indicate if they believed they understood its content. They also answered questions about their educational and other ICT experience. The cassettes with initials and numbers along with capillary tubes, laboratory coat, gloves, and data sheet were provided in a work space separate from other testers and with guidance from the written instructions. Informed consent, and signature into a study log were obtained.

Testers were three practicing physicians, three nurse/nurse practitioners, two medical students and one medical resident. They were not experienced with this type of ICT using whole blood or this cassette. None worked as a certified laboratory technician. They were selected to reflect categories of potential users of this test who were unfamiliar with and unskilled with this test. Each tester took the University of Chicago blood-borne pathogens and universal precautions training to work with whole blood, and their competence in understanding and using this material currently was formally documented for the study, as was TRB-required. The ability of three groups of testers with different clinical roles to read the instructions, to perform the test in accordance with the instruction, and to distinguish negative and positive results at the pre-established limits of detection were evaluated.

### Test and Analysis

The ability of three groups of testers with different clinical roles to read the instructions, to perform the test in accordance with the instruction, and to distinguish negative and positive results at the pre-established limits of detection were evaluated. This was done to determine whether the instructional material was adequate to teach unskilled users in three different practice type settings. Familiarity with universal precautions and biosafety instruction precautions was documented as described above. The fourteen samples, half positive and half negative were tested by each tester in a blinded manner. The tester and one other reader read the tests. The testers read the cassette at 20-30 minutes after sample followed by buffer were applied to the cassette, and recorded their interpretations on a data sheet. Photographs of the cassettes were taken using smart phones with photography in an area with some natural light. The photographs were provided to the tester to read in a blinded manner. One of the testers was a second reader for 7 of the photograph sets and a laboratory scientist was the photograph reader for the remaining two tester's photographs including the tester who was the photographer and second reader for the first 7 testers/readers. A skilled physician scientist with familiarity with the test was a third independent photograph reader. The tester, another unskilled reader (other than the laboratory reader who was experienced), and experienced reader all independently read the photographs of the cassette in a blinded manner and their results were recorded. Data from the data sheet then were analyzed.

Study 7. Detection of acute infection and seronegatvity in Quindio, Colombia by using ICT. Sera from 22 patients who had recently acquired their T. gondii infection in Quindio Colombia and 12 patients who were seronegative had sera that were tested with Vidas ELIFA IgG, IgM and with ICT in their Quindio Reference laboratory.

Study 8. Additional NCCCTS patients and their families had testing with ICT while at follow up visits in Chicago to add data to determine that antibody present for many years is still detectable by ICT. Between March and December 2018 20 participants in the NCCCTS whose serologic status was known from earlier reference laboratory testing and family members traveled to Chicago. Number of tests were 20 seropositive and 8 family members or other controls for separate studies such as multimodal neuroimaging studies were found to be seronegative but did not have other reference laboratory testing. Time from acquisition of infection was noted, in a similar approach to earlier studies of Begeman et al [24] and Lykins et al [23].

Study 9. Use of ICT for Epidemiologic study in Cincinnati. Sera and demographic data from a maternal-infant cohort in Cincinnati were available for 265 women; 264 had data on the variables of interest. Variables of interest included residential address (longitude and latitude), age, education, race, income and pet ownership as part of the original cohort study. Sera were tested with ICT and if positive then were tested with IgM and IgG western blots at LDBIO. A logistic regression model on the results for the 264 samples was used to estimate the ICT Toxoplasma infection positive status by including independent variables such as, maternal age, marital status, Neighborhood Deprivation Score (a higher value means more deprived and missing values are extrapolated from 5 nearest neighbors), latitude, longitude, race, i.e., White or not, and an interaction term between maternal age and Marital status.

Study 10. Evaluation of lateral chromatography test AdBio that detects anti-T. gondii IgM and IgG separately. To determine whether a USA made immunochromatography test (called ADBio) would function as well as the ICT or whether samples from Colombia that had very high performance with ICT would function as well with a different USA made test, an additional set of known IgM positive or IgM negative samples was tested. The tests that were used were the VIDAS (Quindi\acute{ } o, Colombia) test and another commercially available but not FDA cleared or CLIA waived test that detects Toxoplasma specific IgM in the Colombian Reference laboratory.

A total of 147 serum samples were included, selected from the biobank of past studies at the University of Quindio in Armenia, Quindio, Colombia. All samples were previously tested using the reference test VIDAS (VIDAS Toxo-IgG Avidity kit; BioMerieux, Marcy-l'Etoile, France). Samples were divided into the following three groups as defined by VIDAS testing: (1) IgG negative and IgM negative (n=65), (2) IgG positive and IgM negative (n=55), and (3) IgG positive and IgM positive (n=27). These groups corresponded to seronegative samples, chronic Toxoplasma infection, and acute Toxoplasma infection, respectively. VIDAS TOXO IgM (TXM) assay, is an enzyme-linked fluorescent immunoassay—ELFA—(Biomerieux, Marcy-l‘E’toile, France) performed in an automated instrument. In VIDAS IgM test, a value is generated for each sample by forming a ratio from the relative fluorescence of the sample to that of the calibrator or a stored calibrator result (“stored standard”). Test values from patient and control samples are compared to a set of thresholds stored in the computer.

The thresholds and interpretations are given as follows according to the values of the cutoff index (COI): <0.8 COI=non-reactive; 0.8≤COI <1.0=gray zone and ≥1.0 COI=reactive. The AdBio Test (CTK-Biotech, San Diego, CA) uses lateral-flow immunochromatography with a test and control band and goes further with separate bands to differentiate between Toxoplasma-IgG and Toxoplasma-IgM antibodies. Tests were performed and results were read according to manufacturer guidelines and confirmed by three investigators. Sensitivity, specificity, positive predicted values (PPVs), and negative predicted values (NPVs) were calculated against VIDAS IgG and IgM test results, respectively, using the Vassar Stats Clinical Calculator.

Analysis 11: Representative Case Summaries illustrative of practical clinical problems where solutions are needed and potential utility of ICT. Representative case vignettes with concepts they illustrate were collected and were summarized to illustrate impact and need of this method and its historical context (as illustrated in “Studies 2a, b, and 10”).

These brief case summaries are from The National Collaborative Chicago-Based Congenital Toxoplasmosis Study and Consultations to the Toxoplasmosis Center and Toxoplasmosis Research Institute. They illustrate representative, frequent clinical problems incurred from false positive IgM tests. Representative examples of benefit and novel utility of ICT in addressing this problem are also presented. A case summary also presents use of ICT for early detection confirming pre-conception infection when sequential samples obtained in the context of in vitro fertilization (IVF) were available.

### Results

Study 1, Feasibility Clinical Trial Study Performed Exactly as the Test would be Used in Practice, 2020 to 2021, Demonstrates Feasibility, Identifies False Positive Predicate Test Results and Develops New Paradigm to Help to Obviate that Problem.

A flow chart showing the design and historical context of this study is in FIG. 4.

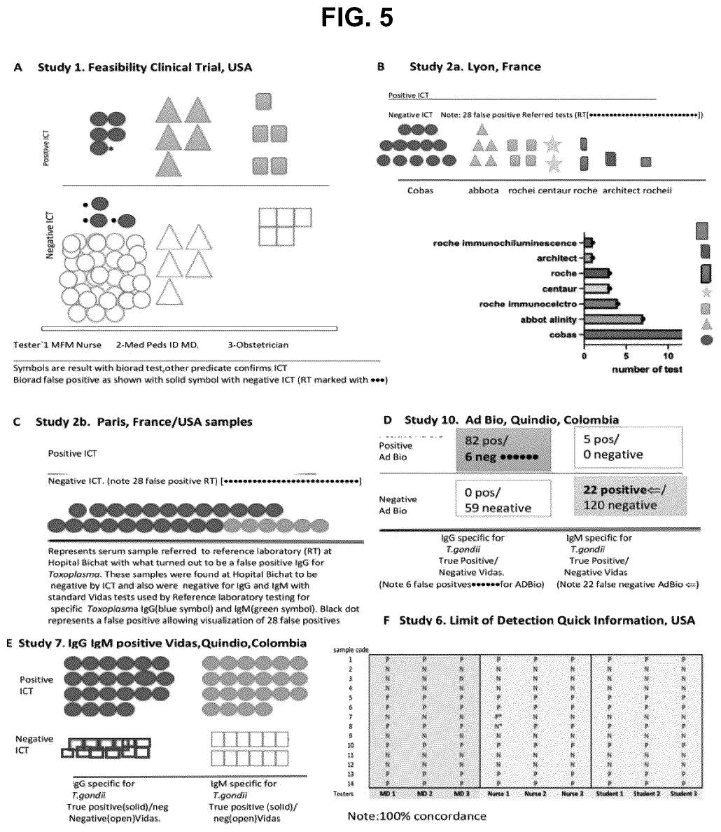

Individual results in the ICT and the predicate test in this ongoing clinical trial study in the context of this design with each of the testers represented by a different color or symbol are in FIG. 5A and Table 1. In addition to Study 1, Table 1 summarizes Studies 2, 3, and 4. Study 4 detail is also in Table 2. Between August 2020 and December 2021, we performed this prospective clinical feasibility trial, Study 1, in which 43 seronegative and 15 seropositive persons were tested with the ICT using whole blood and sera. The 58 sera were also tested with the predicate test used by The University of Chicago Medicine Clinical Serology laboratory, the Automated Bio-Rad assay.

TABLE 3
(called Table 1 in the original PNTD paper and as in this section of this specification). Overview of Chicago formal feasibility
implementation, Lyon referred false positives study, and Lyon Architect and Quindio Vidas G and M back up testing studies
of Chicago monthly screening study, and comparison of red bead (pink line) and black bead (black line) test kits

| | | | | | | Line data |
|---|---|---|---|---|---|---|
| Title | Participant | Positive/ | | POC | | Study no. |
| (Study No.) | number | Negative | Testing site | tester | Laboratory | and/or FIG no. |
| Chicago formal | 1-38 | 5/33 | Hyde Park, Chicago while waiting for | K L | A A, V T, Y Z, back up | Table A in S1 |
| implementation | | | MD, Outdoors drive by car, home visit, | | for US, and Lyon | Commentary *; |
| feasibility | | | Pediatric and Obstetrical practice, | | Reference | Study 1 |
| clinicaltrials.gov (1) | | | Examination areas | | laboratories | FIG. 2A |
| | 39-49 | 5/5 | Hyde Park home outdoor site, Pediatric | S S | A A. V T, Y Z | |
| | | | and Obstetrical practice, Examination | | | |
| | | | areas, Conference rooms | | | |
| | 50-60 | 5/6 | Obstetrician office in obstetrical practice | M S | A A (V T) Y Z, back up | |
| | | | | | for US and Lyon | |
| | | | | | Reference | |
| | | | | | laboratories | |
| Croix Rousse, False | L1-32 | 0/32 | Community local Referring Laboratories | local lab | Croix Rousse | Table B in S1 |
| positives referred to | | | | | Reference | Commentary **; |
| Lyon (2) | | | | | | Study 2A |
| | | | | | | FIG. 2B |
| Chicago monthly | C-S 1-20 | 0/188 | Chicago Medicine Obstetrics Practice | J L, R Mc, | Croix Rousse | Table C in S1 |
| screening study, Lyon | 188 tests | | | K L, Y Z | Reference | Commentary ***; |
| architect, Quindio Vidas | | | | | | Study |
| IgG, IgM (3) | | | | | | |
| Pink/Black (4) | 1074 tests | 623/451 | Ste Etienne, Marseille, Lyon Laboratories | | Ste Etienne, | Table 2****; |
| | | | | | Marseille, Lyon | Study 4 |
| | | | | | Laboratories | |

Legend for Table 1.
* Table A in S1 Commentary. Study 1. Design and Data for 3 Testers with 5 Sera-Positive Persons for each tester, Each in Three Settings with Results Showing Their Primary Data in the Chicago Clinical Feasibility Implementation Trial 2020 to 2021. Corresponds to FIG. 2A. This Study is Performed in Accordance with FDA and CLIA Guidelines and Regulations.
** Table B in S1 Commentary. Study 2 Part 1 Lyon Reference Laboratory ICT Test Results for Sera from Pregnant Patients Referred by Local Physicians for T. gondii IgM with Predicate Tests in Local Laboratories and Negative Western Blot as Gold Standard Comparator. Organization and data correspond to FIG. 2B.
*** Table C in S1 Commentary. Study 3 Shows Concordance of ICT Results in Chicago Acceptability of Monthly Testing In Testing of Sera in Lyon Reference Laboratory Using Abbott Architect and for one person VIDAS IgG ELISA, and VIDAS G and M ELFA in Quindio Reference Laboratory. Initial tests in earlier months were all concordant with Abbott Architect and reported in Lykins et al [28]. This study corresponds to FIG. 4 which shows results of USA Acceptability, Study 3.
****Table 2. Study 4 Shows comparability and high performance of pink and black line (red and black beads) test kits. Line data are at: DOI: 10.17632/dkbzbntpbw.1. Abbreviations are the initials of testers; fp (false positive when compared to gold standard testing).

TABLE 4
(called Table 2 in original PNTD paper and in this
part of specification shown below.
Table 2 Study 4 shows validation of the black version of the
kit in head-to-head comparisons with pink version studied earlier.

| | |
|---|---|
| N of samples | 1074 [symbol] |
| Nature | Chosen, French |
| Predicate | Pink variant |
| Pos/Neg | 623/451 |
| Sensitivity | 99.0% [95CI 98.2-99.8%] |
| Specificity | 100% [95CI 98.9-100%] |

[symbol]

1074 samples included samples from Lyon (N = 200, Wallon, Chapey), Marseille (N = 200, [symbol] ), Ste Etienne (N = 374, [symbol] ), LDBIO Lyon (N = [symbol] )
There was separate additional comparative testing taking place concurrently in Chicago. There was [symbol] concordance. Any discrepancies were resolved with Western Blot.
Line data are at DOI [symbol] . This is part of formal CE Mark, French Laboratories and [symbol] DA/CLIA submissions of line data that also were reviewed in Chicago with concordance similarly documented. Architect G [symbol] M [symbol] also performed. [symbol]
indicates data missing or illegible when filed

All negative results for the ICT and Bio-Rad predicate test were concordant. This is illustrated by the open symbols below the horizontal line in FIG. 5A. Any ICT positive results resulted in testing of the sera in Reference Laboratories.

Results obtained by all readers for the ICT were uniformly concordant with true positive results found in the Reference laboratories. The specificity, sensitivity, NPV, and PPV were 100% o.

AUC was ˜1. Integration into workflow and acceptability were strong. However, a study related Bio-Rad Bioplex 2200 automated IgM EIA problem arose with this NRL predicate test.

Specifically, results of the ICT, as shown in FIG. 5, always had at least two independent observers to insure objectivity in their interpretation as well as to avoid any possible bias. Readings were documented by photographs 20-30 minutes after applying the sample and the buffer immediately after the sample. RMc always read the results independently after initial readers and never influenced the readings of the initial readers. Results of all readers were uniformly concordant. Any positive results were re-tested in Reference Laboratories.

As specified by the FDA, and as shown in FIG. 5A the three testers of different professional backgrounds who might be among the categories of users of the test included an obstetrician, an infectious diseases specialist, and an obstetrics maternal fetal medicine nurse. All who tested participants were blinded to the participants serologic status.

As shown in FIG. 5A and Table 2, each tester tested at least 5 persons who turned out to be seropositive and at least 5 persons who turned out to be seronegative. The different sites for testing in Chicago shown in FIG. 5A and Table 1 included triage area and nurses examining area, the obstetricians examining area/office and sometimes in outdoor field settings during the SARS CoV-2 pandemic. Feasibility, accuracy, and one hundred percent correlation between the whole blood point of care test, the ICT with serum performed by a laboratory member, and a predicate and when needed gold standard test were identified. Practical problems with the predicate test and USA health care system that arose in this USA Clinical Trial feasibility Study 1 demonstrate a compelling need for novel, practical solutions to enable gestational screening to be implemented into the workflow of USA obstetrical practices.

Within testing the initial 6 pregnant participants, we encountered false positive results for two participants in the IgM predicate test and three others later revealing a false positivity rate of 10%. Contacting Bio-Rad about their cut off value for a positive test, re-standardization of the automated machine, checking all reagents did not correct these false positive values above the cut off requiring reporting of a positive predicate test in this practical setting. Further, finding these frequent false positive predicate test results was disturbing for patient participants, providers, and investigators. Correcting the erroneous predicate test data in this Study 1 with follow up gold-standard testing was time consuming, costly, and would result in delays in care in a real-world setting. The prior 1998 FDA advisory was cautionary but the confounding false positives were initially unexpected. This was because we had earlier used US and French reference laboratory backup testing. We had incorrectly considered that the FDA clearance of the predicate test that we were required to use for this clinical trial would have addressed and solved the problem of false positives.

In this real-life practical model in Study 1 that the IRB and FDA required the resource Reference laboratories in the USA and France provided high quality results providing an excellent solution to the problem of the false positive results, we encountered but they had associated substantial delays, cost and inconvenience. In this Study 1 to define practical functioning of the ICT, this problem with false positives occurred while we as investigators promptly saw the negative ICT result in whole blood and sera testing. We knew the results in the context of our earlier data showed very high performance, sensitivity, and specificity of the ICT with Reference laboratory back up. Our earlier work had demonstrated all negative results were accurate with the ICT.

NRL erroneous predicate test results considered to be positive occurred earlier when samples were referred to the USA reference laboratory (27). This occurred when 33 patients with 60 sera were tested in our earlier work [27]. As our Study 1 enrollment reached the first 6 participants with 2 false positives identified by the US and Lyon France Reference laboratories, we recognized that if we could not solve this problem of false positive predicate tests, this de-railed the research study and its longer-term goal of proper testing in systematic gestational screening programs for the USA, and as a model for other countries. For the USA, along with their present high costs in the USA, false positive tests also are substantially harmful.

New Paradigm Elucidated by this Experience

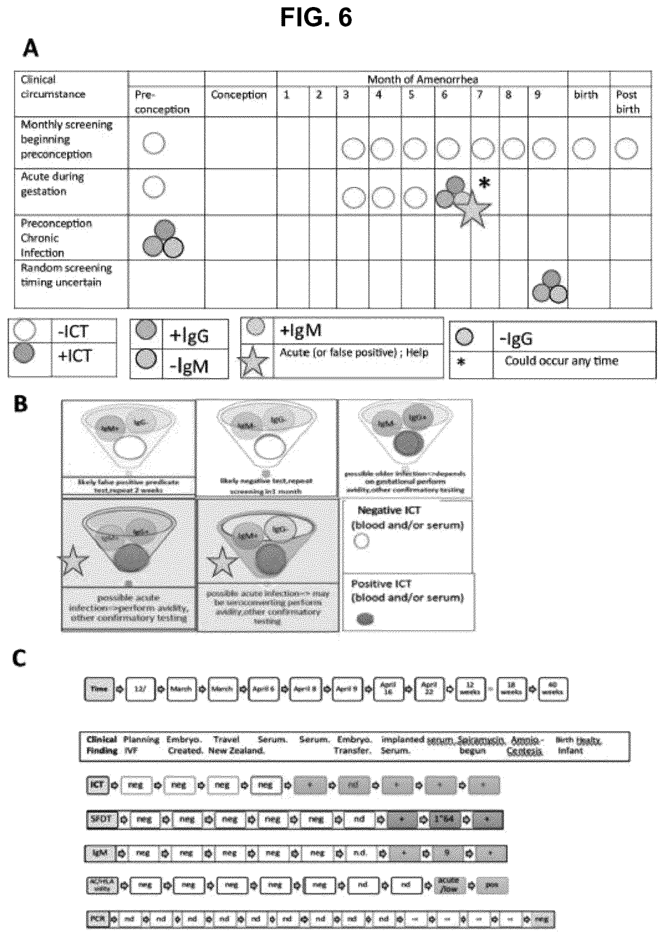

Thus, as we tested the whole blood and sera with the ICT in parallel with testing the sera with the Bio-Rad test, we developed an easy, inexpensive, paradigm shifting approach to solve this problem of false positive tests for the USA. This approach showed the ICT could help to eliminate the problem of false positives both in the clinic and the clinical laboratory. This model (FIG. 6) was to have a method for diagnosis with a test that meets WHO REASSURED criteria available promptly at the time the test was performed and to have a first backup of positive results in serum rapidly in the local laboratory. Our experience shown demonstrates that this ICT performs properly in clinical practice and field studies. We noted that it could be used correctly by previously untrained professional observers, meeting WHO REASSURED criteria.

We found that this also could help to obviate the difficulties caused when a commercial predicate test has false positive result. This was while introducing a novel test that could be low cost and easy to use in the clinic. It became clear from the experience and data presented that this novel test and paradigm could be a useful new method for the clinical laboratory to identify true positives rapidly for this emergent problem/disease. This could help to determine whether there was need for further screening. It could help to clarify whether there was need for emergent care with life, sight, cognition saving medicine that should be initiated promptly while waiting for backup reference laboratory confirmation of a suspected true positive test (FIG. 6).

Study 2 with additional testing of erroneous false positive local predicate tests with ICT and gold-standard testing in reference laboratories demonstrates utility of the novel approach with ICT. Then, Lyon and Paris Reference laboratories' identified erroneous false positive results reported for samples referred for testing from local private laboratories that used commercial tests.

Thirty-two samples that were referred to the Lyon reference laboratory from September 2021 to February 2022 from private laboratories because of the detection of isolated IgM in the course of monthly prenatal retesting, which the main system used in France in this context (FIG. 5B and Table 1): The tests that had been used included Cobas Roche (n=21), Abbott (Architect n=1 or Alinity n=7), Siemens (n=3) (FIG. 5B and Table 1). None of the additional 32 samples gave positive results with ICT or in the reference laboratory with Abbott Architect despite the erroneous reports of positive IgM results (Table 1). Further, in Lyon France, none of the 32 false positive IgM tests with the predicate local laboratory tests used had false positive test results with the ICT or gold standard Western Blot (Table 1). This was using the same referred serum that was tested and reported to be positive from the local private laboratory. These results are included in Table 1, and Box 2 and FIG. 5. Data summarized in Box 2 and Table 3 place the results in the Clinical Trial and monthly screening acceptance studies (Study 3), in the context of other ongoing studies and our earlier published work. Box 2 and Table 3 address details of studies in the USA and elsewhere. Box 2 and Table 3 also collate and address studies of false positive IgMs referred to reference laboratories in the USA and France. Box 2 collates all these studies including those of other countries as a summary of all available results. Sensitivity, specificity, confidence intervals and details of studies are in Box 2 and, Table 3. Performance of the ICT is high, sensitivity 97.5%, specificity 99.4% (serum and/or blood).

Additional testing of a set of samples with Architect, Vidas and other tests was performed in an additional matrix analysis in Study 3 and is pertinent to consideration of false positive test results. Back up testing of another set of sera from a monthly screening and acceptability of the monthly screening program was performed in the Lyon France and Quindio Armenia Colombia Reference laboratories. In these reference laboratory settings as well as in the Paris Hospital Bichat reference laboratory [30] false positive test results were less problematic (FIG. 5C and Box 2, [30]) than in the clinical trial. There were no false positives in the Quindio laboratory Vidas testing and only one patient with multiple consecutive IgG false positives in the Lyon Architect tests.

Study 2 with additional testing of erroneous false positive local predicate tests with ICT and gold-standard testing in reference laboratories demonstrates utility of the novel approach with ICT. Then, Lyon and Paris Reference laboratories' identified erroneous false positive results reported for samples referred for testing from local private laboratories that used commercial tests. Thirty-two samples that were referred to the Lyon reference laboratory from September 2021 to February 2022 from private laboratories because of the detection of isolated IgM in the course of monthly prenatal retesting, which the main system used in France in this context (FIG. 2B and Table 1, and Table B in S1 Commentary): The tests that had been used included Cobas Roche (n=21), Abbott (Architect n=1 or Alinity n=7), Siemens (n=3) (FIG. 2B and Table 1, and Table B in S1 Commentary). None of the additional 32 samples gave positive results with ICT or in the reference laboratory with Abbott Architect despite the erroneous reports of positive IgM results (Table 1). Further, in Lyon France, none of the 32 false positive IgM tests with the predicate local laboratory tests used had false positive test results with the ICT or gold standard Western Blot (Table 1, and Table B in S1 Commentary). This was using the same referred serum that was tested and reported to be positive from the local private laboratory. These results are included in Table 1, and Table B in S1 Commentary, and Box 2, and FIG. 2.

Box 2. All Studies with ICT

Box 2 (part 1)
Box 2. All Studies with ICT

| | | | | | Number of | | |
|---|---|---|---|---|---|---|---|
| | | | | Nature | samples | | |
| | | | Place of | of | Total | | |
| Study | Author 1 | Year [symbol] | Study | samples | (pos, neg) | Sensitivity | Specificity |
| [symbol] | [symbol] | 2023 [symbol] | USA/ | serum/ | [symbol] | 100%/ | 100%/ |
| | | | France | [symbol] | | 100% | 100% |
| | | | | | | [symbol] | [symbol] |
| [symbol] | Abraham | 2023 | France | serum | Total: 3384 | 98.2% | 99.8% |
| | | [symbol] | | | [symbol] | | |
| [symbol] | Zhou, | 2023 | USA/ | serum | Total: [symbol] | NA | 100% |
| | [symbol] | [symbol] | France | | | | |
| [symbol] | [symbol] | 2023 [symbol] | USA, | serum/ | Total: | 100%/ | 100%/ |
| | | | [symbol] | [symbol] | 196/ [symbol] | 100% | 100% |
| | | | France | | | | |
| [symbol] | [symbol] | 2022 [symbol] | Colombia, | serum/ | Total: | NA* | NA* |
| | | | Panama | [symbol] | [symbol] | | |
| | | | [symbol] | | | | |

| | | | | | | Specificity |
|---|---|---|---|---|---|---|
| | | | | Number of | | [symbol] |
| Study | PPV | NPV | AUC | false IgM | Test used | IgM |
| [symbol] | 100%/ | 100%/ | [symbol] | [symbol] | [symbol] | 100% |
| | 100% | 100% | [symbol] 0.991 | | | |
| | [symbol] 99.8% | [symbol] 97.6% | | | | |
| [symbol] | 98.2% | [symbol] | 0.99 | [symbol] | [symbol] | 100% |
| [symbol] | NA | 100% | NA | [symbol] | Architect [symbol] | 100% |
| [symbol] | 100%/ | 100%/ | 1 | [symbol] | Architect, VIDAS, | 100% |
| | 100% | 100% | | | [symbol] W3 | |
| [symbol] | NA* | NA* | NA* | NA* | [symbol] | [symbol] |

[symbol]

indicates data missing or illegible when filed

Box 2 (part 2)

| | | | | | | | | | | | | | |
|---|---|---|---|---|---|---|---|---|---|---|---|---|---|
| [symbol] | [symbol] | 2021 | Morocco | serum/ | Total: | [symbol] / | 99.5%/ | 99.1%/ | 98.3%/ | 0.98/ | NA | [symbol] | [symbol] |
| | | [symbol] | | [symbol] | 349/ [symbol] | [symbol] | [symbol] | [symbol] | [symbol] | [symbol] | | | |
| [symbol] | [symbol] Abdallah | 2021 | [symbol] | serum | [symbol] | 100% | 73.3% | 85.7% | 100.0% | 0.867 | [symbol] | [symbol] | [symbol] |
| | | [symbol] | | | | | | | | | | | |
| [symbol] | [symbol] | [symbol] | [symbol] | serum | Total: [symbol] | 100% | 98.3% | 99.4% | 100.0% | 0.98 | [symbol] | [symbol] | 100% |
| [symbol] | [symbol] | 2018 | Chicago | Serum/ [symbol] | Total: | 100%/ | NA/NA | 100%/ | 100%/100% | [symbol] | NA | [symbol] | NA |
| | | [symbol] | Morocco | | [symbol] | 100% | | 100% | | | | | |
| [symbol] | [symbol] | 2017 | France | serum | Total: 1002 | 100% | 98.7% | 97.4% | 100.0% | 0.993 | [symbol] | [symbol] | 91.3% |
| | | [symbol] | | | [symbol] | | | | | | | | |
| [symbol] | [symbol] | 2017 | Chicago | serum | Total: [symbol] | 100% | 100% | 100% | 100% | 1 | NA | [symbol] | NA |
| | | [symbol] | | | | | | | | | | | |
| [symbol] | [symbol] | 2017 | France | serum | Total: 400 | 97% | 98% | 98% | [symbol] | [symbol] | NA | Architect | NA |
| | | [symbol] | | | [symbol] | | | | | | | | |
| [symbol] | [symbol] | 2023 | [symbol] | [symbol] | [symbol] | 99.2% | 99.0% | 99.0% [symbol] | 97.5% | 0.991 | [symbol] | see above | see above |
| | | | | | | [symbol] | [symbol] | | | | | | |
| [symbol] | [symbol] | 2023 | [symbol] | [symbol] | [symbol] | [symbol] | [symbol] | [symbol] | [symbol] | [symbol] | [symbol] | see above | see above |

[symbol]

indicates data missing or illegible when filed

Box 2. (continued). Legend. ^{a} n this column, the numbers in the green font in parentheses indicate the Study number in this manuscript. In this column, the numbers in the blue font in brackets indicate the Reference number in the literature already published.^{b} In this column, the numbers in the black font indicate data for ICT with serum. In this column, the numbers in red font indicate data for ICT with whole blood from finger prick.^{c} For calculations: TP=true positives, FP=False positives, FN=False Negatives, TN=True Negatives; Sensitivity=probability that a test will give a correct answer, TP/(TP+FN)×100; Specificity=TN/(TN+FP)×100; Positive Predictive Value, PPV=TP/(TP+FP)×100; Negative Predictive Value, NPV=TN/(TN+FN)×100; AUC=(sensitivity+specificity) divided by 2 (where sensitivity and specificity range from 0-1); NA, data not available.*In Colombia, in this acceptability study, in this published manuscript, the comparison was between result in whole blood obtained by finger prick ICT and serum tested with ELECYS-CLIA performed in clinical practice by the clinics. Total:783 (366; 417) whole blood, compared to results from 413 (199, 214) sera tested for those women who returned for serum test. Sensitivity was 96.3% and specificity was 95.9% for ELECYS IgG in this subset of sera. Serum was not tested with ICT. IgMs in sera were also tested with ELECSYS-CLIA IgM performed in clinical practice. Three discordant results between sera and whole blood samples were also tested with VIDAS ELFA G and M. Two acutely infected pregnant women were identified and treated. Using these tests and prior testing results available from earlier testing in clinical practice and follow up of the pregnant women and infants, persistent IgM from infections pre-conception and natural IgM antibodies also were identified. These data are not included in the tabulated overall data analyses because of the differences in study purposes, approaches and methodology, and the absence of serum ICT. Studies in Cincinnati and Panama/USA also had differences in purpose, without any or full confirmation with another Reference test method or substantial methodologic differences. Thus, they also could not be included in the overall calculations. The bolded total numbers are from those samples included in the overall calculations for sensitivity, specificity, NPV, PPV and AUC in Box 1. The performance in the USA hospitals/Reference Laboratory, including field settings, were slightly higher (˜100%, 100%, 100%, 100%, 1) and also in the two French Reference laboratories.* In the south of France tabulated with Study 1, concordance for pink line, black line and Abbott Architect IgG and IgM for 774 sera and additional comparison of testing using pink-line and black-line kits for the same additional 300 samples, without knowledge of standard lab serologic results, was >99% (please see foot note to Table 2 for 2×2 Table). Sera from 44 persons who were seronegative for T. gondii with standard tests and who had malaria, had no false positives test results with ICT.

The following lists tests utilized which have links to full details in Google. All other than LDBIO tests CEMark with applications pending FDA review are FDA cleared other than Architect IgM: Palo Alto Medical Foundation Remington Specialty Laboratory Adult and Infant panels, PCR, and Avidity; ABBOTT Architect IgG, IgM; Biomerieus Agglutination (IgG only), BIORAD PLATELIA ELISA IgG, IgM: BIORAD 2200 Bioplex ToRC; ELECYS ROCHE IgG, IgM; LDBIO IgG/IgM ICT; LDBIO Western Blot II IgG, IgM; LIASON IgG, IgM; VIDAS IgG, IgM, ELFA; VIDAS IGG IGM ELISA; with the manufacturers performance evaluations included. The use of these tests with high correlation with ICT is shown by region and test used in that region in the inset into this footnote that follows. The hand-written inset was written at a meeting with the FDA providing the instructions for feasibility efficacy studies in different settings with numbers of participants required for 510K “clearance” and CLIA “waiver” to be achieved.

Overall, backgrounds of testers included in the 6 nurses, 7 physicians with varying backgrounds 4 medical students, 1 medical resident, as well as laboratory personnel. Earlier studies involved additional testers, including those in other countries besides the US. These included many nursing and medical students, nurses, obstetricians, other students and skilled laboratory scientists. Settings included the following locations: obstetrical triage, standard general obstetric and Maternal Fetal Medicine practice examination settings, pediatric specialty clinic settings, laboratory conference room and office settings, in employee health office settings, in ophthalmology private office setting used by NCCCTS for follow up visits, and during the SARSCovi2 pandemic outside (home backyard, car drive by) settings nearby the University that could resemble a home visit setting. The ICT with serum was read by physicians, laboratory personnel, undergraduate, graduate, and medical students. This was in the Toxoplasmosis research laboratory in Chicago, in reference laboratory settings at Stanford, in reference clinical labs in the US, in Morocco, at field sites, and in France in Lyon, Marseilles, Ste Etienne, and in Paris. In some cases, as in the Cincinnati epidemiology study, the ICT was performed in the LDBIO laboratory in Lyon by RP.

TABLE 5
(original Table 3 in PNTD paper and in text of this
specification below). ICT in US and false positives with comparators.
Table 3. ICT in US and false positives with comparators.

| | Begeman et al. | |
|---|---|---|
| Study | (2017) (1) [symbol] | Lykius et al. (2018) (2)⁵ |

A Summary of Studies that have employed the LDBIO Toxoplasma ICT IgG/IgM
test (ICT) on U.S. sera or whole blood samples, with performance results

| | | |
|---|---|---|
| Comparator test(s) | Sa [symbol] Feldman IgG Dye Test (gold | Abbott ARCHITECT IgG and IgM assays |
| for LDBIO | standard for Toxoplasma [symbol] IgG is the | Abbott North, Chicago, IL, USA) P [symbol] |
| | [symbol] | persons also with earlier dye test and igMi Elisa [symbol] |
| Serum | Yes | Yes [symbol] |
| Whole blood | No | Yes |
| Number of U.S. patients | Infected mother and their newborns, | Positive: 67 [symbol] Negative: 99 |
| represented in samples and | N = 129⁴ Negative IgG and IgM [symbol] 51 | Total: 166 |
| serology results² | Total 180 | |
| | Only 13 were acutely infected at the | Only 3 were acutely infected |
| | time of the analysis in this study. | at the time of the |
| | | analysis in this study |
| ICT Results for | [symbol] P [symbol] NA | [symbol] P [symbol] |
| IgM [symbol] | [symbol] N [symbol] | [symbol] N [symbol] 99 |
| | Sensitivity [symbol] | [symbol] P [symbol] 0 |
| | Specificity [symbol] 100% | [symbol] N [symbol] 0 |
| | | Sens [symbol] 100% |
| | | Spe [symbol] 100% |

| | |
|---|---|
| Overall performances | IgG+/IgM+ 100% (88/88) [symbol] 94.8-100% [symbol] ( [symbol] done |
| of ICT in the US | using Wilson's method with correction of continuity) |
| | IgG+/IgM [symbol] 100% (164/164) [97.1-100%] |
| | IgG [symbol] /IgM [symbol] 99.7% ( [symbol] 06/307) [97.9-99.9%] |

B. Comparison of Toxoplasma [symbol] and comparator predicate tests with [symbol] positive results

| | | |
|---|---|---|
| Source of Sample | Test that generate | Number of FPs People |
| | FP or [symbol] | |
| | “ [symbol] ndeterminate” result [symbol] | |
| PAMF-TSL [symbol] Palo Alto, | IgM ELISA | 33 people |
| California | | |
| H [symbol] spital de l [symbol] | IgM [symbol] see Table [symbol] and [symbol] and [symbol] | [symbol] people [symbol] |
| Lyon, France | [symbol] IgM [symbol] Abbott | |
| | Architect | |
| University of Chicago | Bio-Rad Bioplex 2200 IgM | 3 people |

| | Gomez et al (2018) | Mcleod et al. (2020-present [symbol] see [symbol] website |
|---|---|---|
| Study | (3)⁸ | for clinical trials [symbol] gov NCT044 [symbol] 4132 [symbol] |
| A Summary of Studies that have employed the LDBIO Toxoplasma ICT IgG/IgM | | |
| test (ICT) on U.S. sera or whole blood samples, with performance results | | |
| Comparator test(s) | Sabin [symbol] Feldman IgG Dye | Automated Bio- Rad immuno-assay [symbol] IgG |
| for LDBIO | T [symbol] IgM ELISA | and IgM assays (Bio-Rad Laboratories, Inc., |
| | | Herci [symbol] , CA, USA) Bioplex [symbol] 00 [symbol] |
| Serum | Yes | Yes |
| Whole blood | No | Yes |
| Number of U.S. patients | PAMF-TSL Chronically | Positive: [symbol] |
| represented in samples and | infected: [symbol] | Negative: 43 ( [symbol] ) |
| serology results² | Acutely infected: [symbol] | Total: 58 |
| | Negative: 80 | |
| | IgG [symbol] /IgM+ | |
| | false positives | |
| | from ELISA | |
| | (PAMF-TSL) [symbol] 33 | |
| | Total: 283 | |
| ICT Results for | [symbol] P [symbol] 85 | [symbol] P [symbol] 0 |
| IgM [symbol] | [symbol] N [symbol] | [symbol] N [symbol] 43 |
| | [symbol] P [symbol] 1 | [symbol] P [symbol] 0 |
| | [symbol] N [symbol] 0 | [symbol] N [symbol] 0 |
| | Sens [symbol] 100% | Sens [symbol] NA |
| | Spe [symbol] 99 [symbol] % | Spe [symbol] 100% |
| Overall performances | IgG+/IgM+ 100% (88/88) [symbol] 94.8-100% [symbol] ( [symbol] done | |
| | using Wilson's method with correction of continuity) | |
| of ICT in | IgG+/IgM [symbol] 100% (164/164) [97.1-100%] | |
| the US | IgG [symbol] /IgM [symbol] 99.7% ( [symbol] 06/307) [97.9-99.9%] | |
| B. Comparison of Toxoplasma [symbol] and comparator predicate tests with [symbol] positive results | | |
| Source of Sample | Number of FPs tests | Toxoplasma IgG/M [symbol] CTresult/ |
| | | con [symbol] matory test |
| PAMF-TSL [symbol] Palo Alto, | 60 tests | [symbol] false positive [symbol] |
| California | | |
| H [symbol] spital de l [symbol] | [symbol] M [symbol] G [symbol] | M [symbol] G [symbol] |
| Lyon, France | | |
| University of Chicago | [symbol] | M [symbol] |

[symbol]

indicates data missing or illegible when filed

Legend Table 3. A. ¹Prior to this study in the U.S., two studies in France, one by Chapey et al. [24]* and the other by Mahinc et al., [25] tested the LDBIO test and used the Abbott ARCHITECT IgG and IgM assays as comparator. Both studies were conducted before Begeman et al. [23], but Mahinc et al. [26] was published afterward. Chapey et al. [24] found 13 discrepant results between ICT and Architect, 3 being positive IgM Architect and negative ICT without future seroconversion, hence false Architect IgM results and 13 IgM being positive ICT with negative IgG and IgM Architect but those sera were not controlled using a confirmatory test so they could either be false positive for ICT or false negative for Architect. Such false negative Elisa (Architect and others) results with low titer of IgG below threshold has been described elsewhere [25,39-44]. Positive likelihood ratio and negative likelihood ratio were 97% (95% confidence interval (CI): 91-99%) and 96% (95% CI: 92.5-97.5%), respectively. Mahinc et al. [25], worked with 1002 samples mixing prospective (n=767) and selected samples (n=235). Among the 1002 samples, 13 were false positive with ICT, as proven by confirmatory Toxo II western blot and Isaga (for IgG and IgM, respectively) and patient's follow-up. On the other hand, 32 were false positive for IgM (including 25 selected for false IgM). No false negative for ICT while 2 samples were false negative for Architect IgM.²Samples with either IgG+/IgM−, IgG+/IgM+, IgG−/IgM+according to reference technique as positives. Samples with IgG−/IgM− were considered as negative.³As Toxoplasma ICT IgG-IgM does not discriminate IgG and IgM, IgG+/IgG- samples were not included in this analysis. All IgG+/IgM+, IgG−/IgM+samples (according to reference tests) were considered as positive. All IgG−/IgM− were considered negative.⁴The Begeman [24] paper used duration since birth of an infected child to determine chronic (>2.7 month after birth, N=116) and acute infection (<2.7 months after birth, N=13). IgG and IgM results of positive patients were not available. For positive patients, proof of positivity was obtained because of positive samples (IgG and/or IgM) obtained before ICT. Therefore, sensitivity regarding IgM could not be assessed.⁵ Lykins et al. study [23] also tested 39 Moroccan sera and whole blood samples (33 positive and 6 negative), using the Bio-Rad Platelia Toxo IgG and IgM assays (Bio-Rad Laboratories, Inc., Hercules, CA, USA) as comparators. The ICT (LDBIO) also performed perfectly on these sera, with 33 TP, 0 FN, (false negatives) 6 TN (true negatives) and 0 FP.⁶ 78 patients were tested for both serum and whole blood with 100% correlation. Others were only tested with whole blood.⁷The Lykins paper [23] had 67 positive patients (68 samples) and 99 negative patients (137 samples). However, 64 patients (65 samples) were IgG+/IgM- and not included in IgM analysis. The 3 remaining were IgG+/IgM+.⁸The Gomez et al. study [27] tested a total of 310 patient serum samples. Of these samples, 100 came from the Centers for Disease Control and Prevention Toxoplasma 1998 Human Serum Panel (CDC-HSP). The precise source of these samples is not available. The other 210 samples, from 183 patients, came from patients referred to the Palo Alto Medical Foundation Toxoplasma Serology Laboratory (PAMF-TSL), now known as the Remington Specialty Laboratory. Number breakdown by location is as follows: from CDC-HSP, there were 35 chronically infected patients, 35 acutely infected patients, and 30 negative patients represented. From PAMF-TSL, there were 50 chronically infected patients (50 samples), 50 acutely infected patients (50 samples), 50 negative patients (50 samples), and 33 IgG−/IgM+patients (60 samples).⁹A11 chronically infected samples had IgG+/IgM- (N=85) and were therefore not included in analysis. All acute (N=85) samples were IgG+/IgM+. All 60 false+Samples (33 patients) were IgG- when initially tested with the Sabin-Feldman dye test, whereas they were positive when tested with an in-house IgM ELISA test (Table 3 part B). Follow-up of those patients showed no IgG seroconversion, proving false positive IgM¹⁰Table A in S1 Commentary. Results are as of 31 Dec. 2021.11 All positive were IgG+/IgM−. Four samples were IgG−/IgM+with BioRad, BioPlex 2200 and negative with ICT. They were all proven seronegative at PAMF-TSL and/or Toxoplasmosis laboratory in Lyon, France In Tunisia there were also ˜2 false positives with the ICT.B. +Sabin-Feldman Dye Test/IgM ELISA/PAMF, ACHS, Avidity, IgA, follow-up ++Four test Lyon Panel: BioRad Platelia, Abbott ARCHITECT, BioMerieux [X], [Siemens] Additional data from another site and reported separately. These confirm these results and independently at another site add an additional 30 French patient samples making a total of 99 persons with negative IgG and with one person with a false positive IgM in the presence of IgG. This is a total N of 99 persons whose sera had false positive IgM by other commercial tests where ICT IgG-IgM was negative.

### Current Chicago Study:

Patient 3 (26 Aug. 2020) had CLIA test result IgG “negative” IgM “indeterminate”
Patient 6 (28 Aug. 2020) had CLIA tests IgG “negative” IgM “positive”
Patient 21 (1 Feb. 2021) had CLIA tests IgG “negative” and IgM “positive (1.1)”
Initial outside Siemens IgM test was done in another site and was positive.

Study 4 Comparing kits with pink and black bead and Analysis 4 placing US ICT in the context of other ongoing studies, including Study 3 and previously published studies, demonstrates high performance One thousand seventy-four serum samples were tested with the pink (red bead) line and black line test kits and Abbott Architect in Lyon (N=200; Wallon, Chapey), Marseille (N=200; Lollivier), Ste Etienne (N=374; Flori, Mahinc), LDBIO (N=300; Piarroux, Limonne) with >99% concordance. Any discrepancies were resolved with Western blot. (Links to line data). This is part of the formal CE Mark, French Reference Laboratories, and FDA/CLIA file submissions of line data that also were reviewed in Chicago with concordance similarly documented (Table 2).

Analysis 4 Bibliographical search confirms high performance and that data analysis herein includes all published studies As the ICT is now commercially available following CE Mark approval in Europe, we also used a bibliographic search to attempt to identify results with which we might have been unfamiliar or with inferior performance of the ICT. Box 2 details all studies performed to date: Number of persons (N), Sensitivity (Se)/Specificity (Sp), country of samples, N of false positive IgM, ICT results on false positive IgM and confidence intervals, testing for serum and whole blood are in this Table. To date all studies have involved the authors of this manuscript. There were no additional studies identified that have been reported to date. Table 3A and 3B highlight the data from the USA where multiple sequential studies consistently showed very high performance of the ICT with both sera and blood from finger stick, and studies addressing Mpositivity.

Overall, a total of 4967 sera, 1685 positive and 3282 negative, and 1093 whole blood tests, 362 positive and 731 negative tests have been performed, including all published, ongoing studies and those herein. Additional testing in an acceptability study in Colombia added another 413 whole blood paired with serum samples with high performance not included in the total. 4967 total sera (1685 positive and 3282 negative) and 1093 total (362 positive and 731 negative) whole blood finger prick samples were tested in matrix analyses correct for calculating sensitivity, specificity, NPV, PPV and AUC. There was high sensitivity and specificity, 99.2%/99.0% for serum and 97.5%/99.7% for whole blood (Box 2). Overall NPV is 99.7% (serum), 98.78% (whole blood), PPV is 97.5% (serum), 99.44% (whole blood). AUC is 1 for USA and 0.99 for serum and 0.99 for whole blood for all the tests calculated worldwide. Study 2 a and b and earlier overall performance of ICT with NRL tests demonstrates that false positive IgM also is high Overall, including our own results herein, we found 137 samples with false positive IgM in at least one NRL technique also tested with ICT, among which 132 were found negative in ICT. The specificity of ICT for false positive IgM was 96.4%. Three samples were from IgG negative pregnant persons in Chicago. Two seropositive persons also had false positive NRL IgM, was not found in Reference laboratory IgM tests. In addition 22 false positive IgG and later 6 false positive IgM results were correctly identified by ICT at Bichat-Claude Bernard Hospital, Laboratory of Parasitologie, Paris, France [30](FIG. 5C and Box 2) and 5 false positive IgG identified in the predicate Abbot Architect in the University of Chicago medicine samples tested in Institut des agents infectieux, Hospital de la Croix-Rousse, Lyon, France (Table 1, and Box 2).

Study 5 comparing time, cost of tests and approaches demonstrates ICT time/cost savings and aids in eliminating delays, Approach and an analysis of relative time and cost is in Table 4. The ICT is substantially time and cost saving, Representative case summaries illustrate problems in care that false positive tests can cause and utility in identifying seroconversion in infection acquired prior to conception, called study 4b and “analysis 11” in methods). These provide further evidence of problems that false positive test results cause, and additional practical operational difficulties encountered in the USA.

FIG. 6 (Study 4b) shows this new approach diagrammatically and its exquisite sensitivity and therefore utility in identifying infection prior to conception. In FIG. 6C the ICT detected seroconversion a day earlier than the Sabin Feldman Dye test and IgM ELISA in the USA Reference laboratory. There are a number of examples of patients who developed M alone then M and G [26]. In the Mahinc et al study ([26], Box 2 and Table 3] there were 50 serum samples from 24 women for whom there were 17 samples with IgM only and 33 samples with IgM and IgG; ICT was positive for all samples except one that had a borderline IgM ISAGA of 5 for a patient who later was found to have acute Toxoplasma infection. There were also another 144 acutely infected persons identified in the USA, France, Morocco and Colombia all identified as positive with the ICT [24,25,27,29,30] Box 2 and Table 3. It was unusual, however, to watch seroconversion with as much precision in narrow time intervals so early in infection as shown in FIG. 6C.

Box A contrast the current status and consequences of congenital toxoplasmosis at earlier times in France and continuing to the present in the USA. This illustrates that the ICT and gold standard back up testing can help to solve a substantial health care problem. This is both in a historical context and at present, with potential spillover benefit for care for pregnant women and their families.

TABLE 6
(original Table 4 in PNTD paper and in text of this specification below).
Table 4 Approach using ICT to gestational screening is cost and time saving.
A. Approach to screening

| Serology and timing | Result | Course of action |
|---|---|---|
| Serologic tests pre-conception | Negative | Screen beginning in first trimester before |
| | | 14 weeks [symbol] monthly |
| Serologic tests positive pre- | Positive | Gestational screening not needed |
| conception | | |
| Serologic tests see [symbol] | Negative to Positive | Treat without delay |
| in gestation | | |
| Wild screening positive result | Positive | Expert consultation, often [symbol] |

B. Cost and Time saving just for fingerprick, all sample handling,
reporting and billing for ICT at Point of Care versus Predicate testing

| Parameter | Point of care test | Predicate test |
|---|---|---|
| Time for test | <2 minutes to perform | Days |
| | 20 minutes to read | |
| Location | POC | Requires Lab |
| Time to report | Minutes | Over days Multiple Levels |
| Cost | <$10 | >$500 per test is the USA |
| Participant preference | Yes | [symbol] Ok, but POC is preferred by some [symbol] |
| False positive results | Very rare: high | Not infrequent; problematic |
| | sensitivity and | |
| | specificity | |
| Solves problem of false | Can be very helpful [symbol] also | Can cause problems in clinical lab |
| positives | as back up test in lab | |

[symbol]

indicates data missing or illegible when filed

Study 6, testing ability of written instructional materials to facilitate clinical use of the ICT by healthcare practitioners not skilled in using the ICT in a limit of detection quick information study per FDA and CLIA instructions, demonstrates high performance.

Moving toward implementation, ability of written instructional material to be used in clinical practice with samples at limits of detection for positive whole blood samples and negative whole blood samples was found to have perfect performance. This perfect performance after they read the Quick Information (QI) materials was for all the 9 “blinded” readers and testers (3 nurses, nurse practitioners, resident and 2 medical students, and 3 practicing physicians) who were not previously trained to use this ICT (FIG. 5F and Box 3). It was for all users and all samples when the cassette was evaluated in real time and when the smart phone photographs were evaluated for all 14 unknowns at the limits of detection samples (FIG. 5F and Box 3). There was reproducible, replicable, robust, perfect performance in Study 6, with additional un-trained users reading the Quick information page of instructions as their learning material. In this setting, the ICT also worked perfectly with whole blood at the limits of antibody detection by the 9 users reading test kits in real time and photographs of those tests subsequently.

Legend for Box 3. The Objective of Study 6 was to determine whether multiple relevant users could effectively learn from a “Quick Information (QI)”, single page with two sides, in accordance with FDA/CLIA regulations. A.QI, regulatory processes, and participant eligibility. QI shown in A is not only an instruction page about how to perform a finger prick but part of a specific required experiment germane to Federal regulations. These regulations require a study with experimental testing at the “cut off” limit of detection using this brief stand-alone QI (A). The FDA and CLIA required creation of and testing this QI in 2020, and iteratively reviewed the versions of this QI until it complied with all their guidelines. This review culminated in creation of (A) that was ready to test whether it could be an effective stand-alone learning tool for use of the ICT. Then multiple levels of IRB and research office approvals of this page and study were required. To comply with IRB approval, testers were asked to complete a suite of safety courses and tests providing information about blood borne pathogens and Toxoplasma. Certifications of completion of this course and testing had to be received by the IRB before beginning. Volunteer participants in 3 relevant categories of care providers who might use an ICT in the future were sought by word of mouth. No compensation was provided. An informed consent was signed. A log page with specific information and questions about eligibility was completed. (B) i. Preparation of samples: A serum from a French blood bank was identified after testing the initial serum to know its IU. Then, in France the “cut off limit of detection” dilution was defined. This was a dilution that had to be found when tested to be incorrect one of a hundred times it was tested when characterized at multiple dilutions. Dilutions were tested 100 times each by an experienced (RP) and untrained user. A dilution of 1:89 was defined as the “cut off” dilution to be used to create the positive whole blood samples for the testers. Then, this serum was sent to the United States where the test had to be performed on 14 samples by each of the relevant US care provider personnel in the following study: Whole blood samples, either negative or the negative containing a 1:89 dilution of the French serum were prepared at the “cut off” dilution (called “positive”). Then the samples were aliquoted into separate tubes. Then a random number table was used to assign a number from 1 to 14 to each tube of blood. There was one set of numbered tubes for each tester. Each numbered tube contained either a negative or positive sample. Nine identical sets of tubes with the assigned numbers were prepared with one set for each tester. B,C. i. Testing. After signing an informed consent pre-approved by the IRB (ethics committee), answering a set of questions concerning their background and confirming eligibility, being given a data sheet approved by the TRB, FDA/CLIA to complete, signing a log book, the testers each received a container with materials to use in a designated work space. In this container they received appropriate PPE, a barrier for the work space, a container to dispose their work materials, their set of samples in a small test tube rack, their own data sheet, a pen to record their results, a QI page, cassettes, buffer, marked capillary tubes, and a timer. They were then each able to begin testing these samples in sites approved for this testing and with appropriate PPE for handling human blood samples. The testers did not receive instructions other than their QI instructions. Testers were separated in time and space from each other and could not talk with or watch each other. When they completed their readings and recording of their results, a photographer documented the appearance of the cassettes at 20-30 minutes after loading the cassette with sample according to instructions. In this test, per FDA/CLIA instructions, there was no fingerstick performed. The testing first included two testers who checked the negative and prepared positive initially. They found discrimination of negative and positive samples was readily accomplished. Then, the blinded 9 testers, testing the replicate samples, performed the testing. This was to address who can learn to perform this test easily, reliably and how well with the QI, questions about reproducibility, repeatability, testing of learning and an objective, rigorous proof of results. B, C. ii. Evaluation of photographs: The same blinded reading process of photographs provided to each tester also took place. The only person who knew code for labeling of tubes was the scientist who prepared them. No tester knew the code or what tubes contained. D. Data and analysis: Concordance of tube contents and tester readings of cassettes in real time and photographs was 100%. This provides evidence that multiple users of various relevant backgrounds can learn to use this test with samples with IU at the established limit of detection. This is the US CLIA FDA process, and without it, this test cannot be available in the US. This testing demonstrated that physicians in training, in practice, and nurses can learn to use the ICT in 3 physically separate settings from reading the QI. This study also demonstrates reproducibility and repeatability of testing, in the results at the point of test with the ICT. It is also included to show the relevant user learning and testing in this mandated study, in addition to the QI showing how to do the finger-prick. Additional information in the new “Perspectives” section also presents data from over 1500 finger stick tests with close to 100% accuracy in many diverse settings by many testers in 4 countries with varied backgrounds. In addition to this testing in Study 6, we include information about the wide variety of testers, settings, locations of testing, levels of education of testers. In Conclusion, we demonstrate that it is easy for physicians, physicians in training, and nurses to learn to use a simple carefully designed description of use of the ICT and that this QI is an effective teaching tool. Also, we find that results with the ICT are robust, repeatable, reproducible, sensitive and specific in multiple settings. Box 2, Perspectives section of Discussion contain additional pertinent information and data.

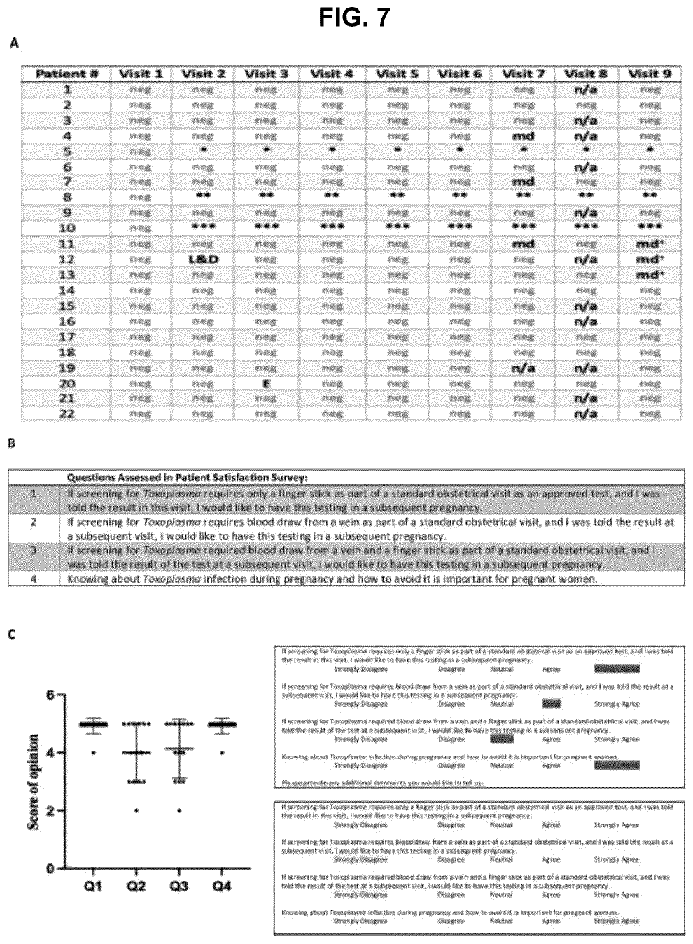

Study 3 demonstrates feasibility and acceptibility of monthly gestational screening with ICT Overview. Early in the work with the ICT in 2017 to 2018 we performed this study to determine whether monthly gestational screening would be feasible in a research study setting (FIG. 7 and Table 1). Results of the testing did not enter standard medical records or the EPIC system at that time and the testing took place earlier than Study 1 but was completed after that study. This study was initiated before the clinical trial and led to the initial meeting with the FDA when a program officer from the Thrasher Foundation emphasized the importance of FDA clearance for the test to be useful to help patients in the USA. We also asked participants at its completion whether these participants felt it was important and comfortable to have knowledge about Toxoplasma in pregnancy and whether they would want serologic testing and/or the finger stick point of care test in subsequent pregnancies if it were approved in the USA. The intent was to determine whether screening might be acceptable in standard academic obstetrical USA practice: Some parts of the study, e.g., the questionnaire and additional backup testing were performed in 2020. Results for the initial tests for the participants' visits were included in the earlier 2018 publication [23]. Thus, numbers included for this study in the cumulative total of tests were subtracted from tests shown in FIG. 7 and for this entry in Box 2.

### Patient Characteristics and Testing Details

Patients were all identified at between 8-12 weeks gestation. Patients had a median age of 31 years (range: 24-40 years). Seven of the 22 participants were nulliparous, while the remainder had been pregnant once or twice before. None reported having been tested for T. gondii infection in the past. Participants were enrolled in the study between September and November, 2017. The study initially concluded in September, 2018 with the birth of the last participant's child and 6-week post-partum visit. Because five mothers were missed by our study group at their 6-week postpartum visit, an anonymized questionnaire was provided in 2022 for those participants. This was considered separately in our analyses. Patients were tested at monthly intervals after their initial enrollment and tested until their 6-week postpartum visit. A small subset of patients (3/22) were withdrawn from the study: One individual underwent elective termination due to fetal anomalies. One participant suffered a spontaneous abortion. The third patient chose to withdraw from the study due to traditional beliefs about dangers associated with venipuncture. No patients (0/22) had evidence of prior infection with T. gondii upon their initial testing with the whole blood POC test, and none seroconverted during gestation. One participant had a faint band in the ICT suggesting the possibility of a positive test on one test, but this was only visible to the naked eye and could not be independently confirmed with photography. Per manufacturer instructions, this test was interpreted as negative. There was 100% concordance between testing interpretations of the POC test and confirmatory testing, including the ARCHITECT/Vidas/Western blot systems and the serum-based POC test variant, commercially available and now CE mark approved in France. The course of gestational screening for each participant is presented in FIG. 7.

Patient perceptions of POC gestational screening are favorable Initial response of patients and their families to educational materials screening was positive. No patient declined to participate. Patient participants asked whether other pregnant and non-pregnant family members and friends could join. Some fathers asked to be tested to know their own serologic status and if they might be at risk of retinal disease if infected. At the end of the consecutive screening tests, we administered the patient preferences survey to an available subset of the cohort (14 in total) at their 6-week postnatal visit. Two of those participating women who were missed completed the questionnaire in 2022. The POC testing and screening for acquisition of T. gondii in gestation was well received by all participants. In the post testing survey 100% strongly agreed with the value of education and testing and viewed the point of care finger stick testing favorably. 100% agreed they would have testing in subsequent pregnancies and advise a friend or family member to do so. Testing by venipuncture and send out testing was viewed less favorably in contrast (p<0.05 at 6 weeks). Specifically, on average, patients appeared to prefer finger-stick POC screening to venipuncture, (POC-4.93±0.27 vs venipuncture- 4.00±1.04) (FIGS. 7B and 7C). More than 100% (14/14) of enrolled patients “strongly agreed” or “agreed” with Question 1 that they would like to have POC testing in a future pregnancy if it involved ICT by finger stick if this did not require monthly venipuncture. In contrast for Question 2, only 9 of 14 (64%) “strongly agreed” or “agreed” with screening with venipuncture (Chi squared equals 6.087 with 1 degree of freedom. The two-tailed P value equals 0.0136). 100% of patients “strongly agreed” that knowledge of T. gondii infection and ways to avoid exposure were important for pregnant women. The two participants who responded in 2022 also agreed that screening was worthwhile but only would want to do this by finger stick and not venipuncture, a similar pattern. (FIG. 7C right panel). In summary, in this post testing survey at 6 weeks post-partum or several years later, 100% (16 of 16) of participants “strongly agreed” or “agreed” with Question 1, having and recommending other pregnant women have finger stick ICT monthly during gestation. In contrast, for Question 2, 56% (9 of 16) of survey respondents “strongly agreed” or “agreed” with having venipuncture and send out testing in subsequent pregnancies and recommending this for other pregnant women (Chi squared equals 8.960 with 1 degree of freedom. The two-tailed P value equals 0.0028). All agreed that education and testing are valuable for obstetrical patients with their providers.

### Provider Perceptions of POC Gestational Screening are Favorable

There was not a formal questionnaire for providers. Rather, level of interest and enthusiasm was reflected by the following: All providers remained involved in the study with their patients. Additional providers in the practice noting and learning about the ongoing study with the initial providers asked to join with their patients. Those still practicing at the University of Chicago at a later time collaborated in the subsequent clinical trial Study 1 presented herein. These objective measures demonstrate provider satisfaction. All providers indicated verbally that they found the finger-stick testing and monthly screening a constructive addition to their practice. The rapidity of obtaining the results was viewed positively. In Studies 4b, 7 and 8, ICT detects early seroconversion and distinguishes additional seropositive and seronegative samples in USA, France and Colombia.

We noted, as shown in FIG. 6, the ability of the ICT to detect very early seroconversion in a study of sera obtained at narrow intervals to monitor hormone levels during in vitro fertilization (IVF), that happened to occur during very early seroconversion. This was a USA patient whose in vitro fertilization had occurred 6 months prior to implantation of their embryo at a time that she was seronegative. In the interval between in vitro fertilization and implantation she had traveled to New Zealand where she likely acquired T. gondii infection in the weeks before implantation as shown in FIG. 6C.

Our earlier studies (Box 2 and Table 3) have all identified perfect performance in detecting sera from those with acute infection in the USA. It was, therefore, of interest to determine whether the same would be found in sera from patients with acute infection with the genetically different parasites found in Colombia. FIG. 5E and Table 5 A-B shows perfect ability, sensitivity, and specificity of the ICT to also identify acute infection (IgG, IgM) in Colombia (N=22) and those who are seronegative (N=12), (p<0.0001)(sensitivity 100% and specificity 100%).

TABLE 7
(original Table 5 in PNTD paper and in text of this specification below)
Table 5. Results of the studies in Colombia with [symbol] G and M [symbol] .
A. Contingency [symbol] of the results between [symbol] IgG [symbol] and IgM [symbol] and [symbol]
samples [symbol] patients [symbol] results in these patients [symbol] A

[symbol]

| | [symbol] IgG/IgM [symbol] (Positive or Negative) | | |
|---|---|---|---|
| [symbol] IgG Results | Positive | Negative | Total |
| Positive | 22 | 0 | 22 |
| Negative | 0 | 12 | 12 |
| TOTAL | 22 | 12 | 34 |

A

[symbol]

| | [symbol] IgG/IgM [symbol] (Positive or Negative) | | |
|---|---|---|---|
| [symbol] IgG Results | Positive | Negative | Total |
| Positive | 22 | 0 | 22 |
| Negative | 0 | 12 | 12 |
| TOTAL | 22 | 12 | 34 |

B. Contingency analysis of results of positivity and negativity for [symbol] IgG [symbol] and IgM [symbol]
between VIDAS and rapid test [symbol] summary of diagnostice properties [symbol] including negative
predictive ( [symbol] ) and positive predictive ( [symbol] ) values. B

[symbol]

| | [symbol] IgG Results | | |
|---|---|---|---|
| [symbol] IgG Results | Positive | Negative | Total |
| Positive | [symbol] | [symbol] | [symbol] |
| Negative | [symbol] | [symbol] | [symbol] |
| TOTAL | [symbol] | [symbol] | [symbol] |

B

[symbol]

| | [symbol] IgG Results | | |
|---|---|---|---|
| [symbol] IgG Results | Positive | Negative | Total |
| Positive | [symbol] | [symbol] | [symbol] |
| Negative | [symbol] | [symbol] | [symbol] |
| TOTAL | [symbol] | [symbol] | [symbol] |

B

[symbol]

| [symbol] | Sensitivity % | Specificity % | NPV % (95% CI)/ |
|---|---|---|---|
| Anti- [symbol] | (95% CI) | (95% CI) | PPV % (95% CI) |
| IgG | [symbol] ( [symbol] ) | [symbol] ( [symbol] ) | [symbol] ( [symbol] ) |
| IgM | [symbol] ( [symbol] ) | [symbol] ( [symbol] ) | [symbol] ( [symbol] ) |

[symbol]

indicates data missing or illegible when filed

This brought the total to 144, as above. Further, serologic status was correctly identified for additional NCCCTS participants tested with finger-stick whole blood and serum between March and December 2018 herein (N=20 positive chronically infected persons [times after infection years were known to be greater than 17 years for all except 3 persons], and 5 negative). We found perfect correlation of testing of whole blood obtained by fingerstick and serum testing in the United States, herein, and almost perfect correlation in Morocco this ICT using whole blood accurately distinguishes seronegative and seropositive status as occurs in seroconversion. In study 6 in which we tested whole blood (that contained serum that originally had 38 UI/ml of IgG and 63.89 ratio for IgM according to Roche Toxoplasma kits, diluted 1:89 in whole blood from a seronegative donor) at the limits of detection in the “QI study”, there was high accuracy in distinguishing positive and negative samples (FIG. 5F). There were N=63 negative and 63 positive, making a total of 126 tests of samples performed. There was 100% accuracy both with the cassette and with photographs read by the tester and two additional readers. All readings were congruent and consistent. All were blinded for 9 testers with 3 testers in each of three settings (physician office, nurse health care setting, and laboratory conference room), and with testers differing professional backgrounds (3 nursing, 3 medicine in training, 3 licensed physicians in practice previously unskilled in use of such a test) (FIG. 5F and Box 3).

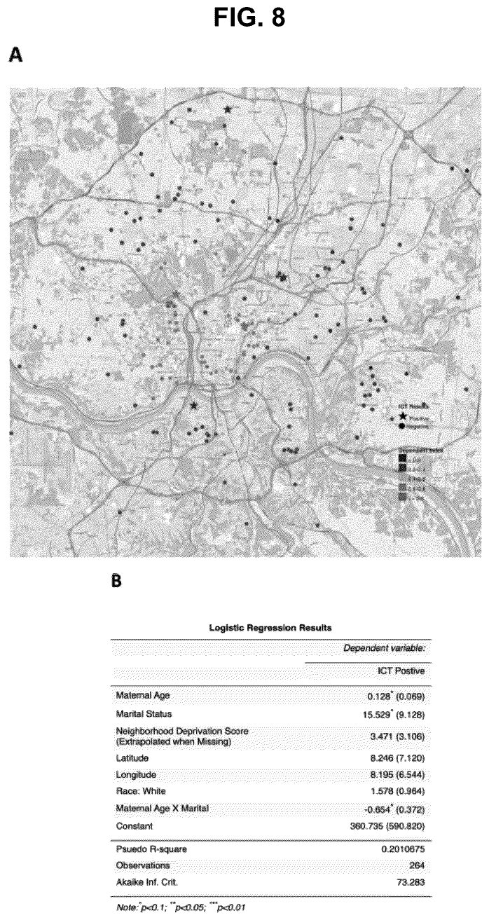

In Study 9, use of ICT for Cincinnati epidemiology study between 2017 and 2019 demonstrates that ICT is an efficient way to perform such studies and that prevalence is low in Cincinnati Of the 265 mothers tested, 8 (3%) had a positive IgG for Toxoplasma infection. None of these had a positive IgM. Variables of interest were available for 264 of the mothers including residential address (longitude and latitude), age, education, race, income and pet ownership as part of the original cohort study. There were no significant associations of testing positive for Toxoplasma infection and any of these variables (FIG. 8).

In Study 10, results with ICT AdBio test (Onsite POC) that detects anti-T. gondii IgM and IgG separately has both substantial false negatives and false positive IgG (9%) and false negative IgM (18.5% true positives detected) We had hoped that a test developed in the USA called ADBio that is purported to distinguish IgG and IgM specific for Toxoplasma might be useful in a field setting. This test had a high proportion of False negative and substantial number of False positive results (FIG. 5D and Table 5). A total of 147 samples were tested using the Onsite POC test (FIG. 5D and Table 5) in Quindio, Colombia. By using Toxoplasma-IgG detection by VIDAS as reference test (FIG. 5D and Table 5), the sensitivity of Adbio for Toxoplasma-IgG detection was 100% (95% confidence interval [CI], 94.4%-100%), and specificity for Toxoplasma-IgG detection was 90.8% (95% confidence interval [CI], 8.3%-96.2%). Of note, in 6 of 65 (9%) samples, the Onsite POC kit tested positive for IgG but were negative by VIDAS IgG. In 5 of these sera the bands observed in AdBio were visible. One serum with positive result for IgG in the AdBio but negative in the IgG VIDAS, was obtained from a boy with confirmed congenital toxoplasmosis who was being treated when the sample was obtained. For diagnostic accuracy of IgM detection, sensitivity for Toxoplasma-IgM detection was 18.5% (95% confidence interval [CI], 7.0%-38.7%; 5 of 27), and specificity for Toxoplasma-IgM detection was 100% (95% confidence interval [CI], 96.1%-100%; 120 of 120). Summary of the diagnostic properties found in this group of sera are shown in Table 5, including NPV and PPV values.

### Summary of Results

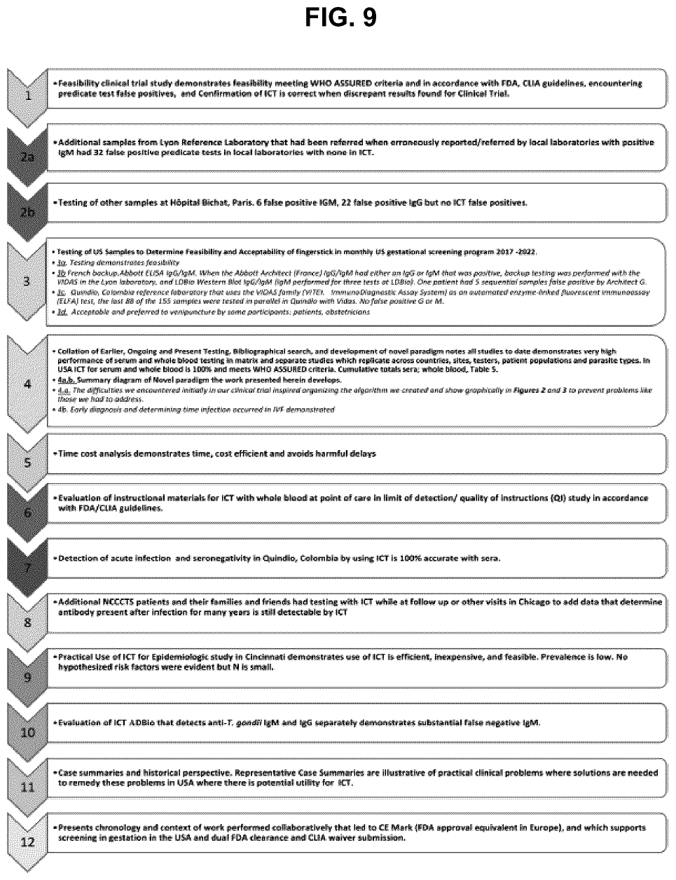

FIG. 9 shows a Summary and flow chart of all Results showing Milestones achieved in this work. This shows the complexity of this decades long work that occurred in stepwise studies. FIG. 9 summarizes the results that corresponds with the Design of the “road map” in Box 1.

### Discussion

Implications of main clinical findings. The results above demonstrate that the ICT has proven effective at identifying sera and whole blood samples of USA and non-USA patients with known T. gondii infection with high performance for each circumstance/patient/participant tested. The ICT recognizes both quadrivalent and pentavalent IgM and bivalent IgG bound by the antigens to the black beads and in a line at T on the chromatographic paper, functioning well for all persons in each site in north and Latin America, Europe and Africa. It detects seroconversion early in infection. It is also effective in identifying the false positive test results for T. gondii specific IgM of other currently FDA cleared tests of sera when no T. gondii specific IgG is present.

It was well-accepted in a monthly screening program that was shown to be feasible in a USA academic obstetrical practice. It also functioned with high precision while meeting WHO REASSURED criteria even in whole blood samples at the limit of detection of specific anti-Toxoplasma antibody. It was found to be straightforward for physicians, nurses and medical students and a medical resident to easily learn to use the ICT and accurately interpret the ICT results using the Quick Information in simple written instructions.

Up through and including the current stages of the clinical feasibility trial at the University of Chicago Medical Center, diagnostic sensitivity has exceeded 99% and specificity has stayed at 100% with all samples of U.S. patients regardless of parasite or patient genetics. In addition, across several of these studies, this ICT has outperformed other NRL screening tests. Herein, out of 99 IgM false positive sample results, across multiple consecutive different USA and French sets of data recently, there have not been false positives or false negatives using the ICT. In addition, in two countries (the USA and Morocco [29]), the ICT has not had false positive or borderline bands when testing serum and/or whole blood in the USA and only very rarely in Morocco with whole blood. While it was already known that this test could perform accurately, this present work also has evaluated the ability of ICT to correct the errors of other carefully tested, commercially available screening assays [27], using prospectively and retrospectively collected sera in the USA, France and Morocco. The high specificity is a particular strength for the ICT IgG-IgM device, especially when compared to other currently available commercial tests for anti-Toxoplasma IgM.

The data from Abraham, Houze' et al (ECMID and [30]) and Mahinc et al [26] increases the number up to 137 of such false positive IgM studied with the ICT. Mahinc et al also studied 23 false positive Architect and/or Bio-Rad Platelia IgM [26]. In the Mahinc study [26], false positive IgM in the Bio-Rad test were obviated by ICT testing 21/23 of the time. In Tunisia [31], recent results were similar adding additional data but with a higher proportion of false positives [31]. Ten of 13 false positives were negative in the ICT. Although there were no ICT false positives in these data sets in the US, the occasional false positives (5 of 36) in the work earlier in Marseilles and Tunisia emphasize the importance of confirmatory testing of positive results. The high-quality performance of some of the Reference tests emphasize that some tests seem to perform better than others, with greater or lesser sensitivity for IgM, when used in Reference laboratories.

Our studies, along with the earlier experience in the Palo Alto reference laboratory and collated recent results, demonstrate practical problems in the US with potential serious consequences for patient care using NRL tests [35] where the ICT can be helpful in a patient's management. This has been confirmed in France making a total of 132 of 137 IgM and 27 of 27 IgG false positive results were corrected. False negatives are uncommon but would be detected by repeat testing in gestational screening programs. Any positive ICT during gestation would have confirmatory testing to differentiate IgG and IgM. The occasional false positives would be detected by back up testing in the reference laboratory in the USA or use of multiple tests including the Western blot in France. Reference laboratory gold-standard testing and certain commercially available test reagents have higher performance than testing in local laboratories [27,30]. The ICT has high precision with samples at the limit of detection. That the test is easy for medical students, a medical resident, practicing board certified physicians, nurses/nurse practitioners, without familiarity with the ICT, to perform and interpret is congruent with a recent experience with 30 practitioners in Armenia, Colombia [47]. This experience was with patients infected with genetically distinctive Colombian parasites [47].

Acceptability in a Colombian patient and obstetrical practitioner group was high [47], similar to acceptability in our USA experience presented herein. Colombian sera also were tested in Colombia [47-50] with a different lateral chromatography test made in the USA called the ADBio. This test differentiates IgG and IgM and has a USA price more than ten times that expected for the ICT. Unfortunately, the performance of the USA manufactured ADBio test was problematic when compared in the Quindio Reference laboratory with Vidas IgG and IgM reference tests [47-50]. This is similar to our earlier results with this test with French, and USA [27], sera. For the Colombian sera specifically, there was a marked difference of the ICT and combined detection of IgG and IgM antibodies: The AdBio test resulted in lower sensitivity for IgM in stored samples from a biobank. ICT combined simultaneous detection of IgG and IgM can improve sensitivity for IgM because most of the IgM sera used for sensitivity analysis already have IgG [26,27,30} and the mechanism of the test with antigen coating the black bead reacting with both quadra/pentavalent IgM and bivalent IgG which react with the antigen placed in the line on the nitrocellulose. This combined detection of different isotypes also contributes to better specificity. The lysate antigen used in the ICT contains many proteins. The Western blot can accurately discriminate between and recognize IgG and IgM specific for T. gondii, as can the combination of other tests such as the Sabin Feldman Dye test which detects IgG and the double sandwich IgM ELISA or the IgM ISAGA. The IgM ISAGA is more sensitive and thus preferable for use for infants.

Interpretation in view of the literature, and additional strengths and limitations Interpretation. In the context of clinical protocols for prenatal Toxoplasma screening, the ICT insures that far fewer “false alarms” are generated and that less time and resources are spent on confirmatory testing for a pregnant woman who shows an isolated positive Toxoplasma IgM test. Risk that such sample may be a false negative IgM from the ICT test is very low, but cannot be excluded. To avoid any risk the patient should be retested for IgG and IgM 2 weeks later to ensure that IgG did not appear. This re-testing for isolated IgM, at this shorter interval, is also part of systematic gestational screening programs. It should be emphasized again that POC tests for anti-Toxoplasma IgG and IgM, such as the ICT, are merely a first step toward diagnosis, given that IgM antibodies can persist for up to several years after acute infection.

For any woman who receives a false positive IgM test result, the next step of an evaluation with other tests can involve weeks of waiting for a blood sample to be tested using technology that runs at much higher costs than the point of care test [1,2,6,7,27,33,35]. Persistence of IgM in subacute or chronic infection, results just above the cut off or more, and true seroconversion all cause positive IgM results. This test does not distinguish persistent IgM and that present with true seroconversion. Ideally, testing is performed initially pre-conception. If testing in gestational screening programs was not performed pre-conception to identify serologically positive persons who would not require monthly screening to detect seroconversion, testing in gestational screening programs should take place no later than during the initial 14 to 16 weeks of amenorrhea, and preferably earlier at the first pre-natal visit. This enables use of the avidity test to attempt to date infection when IgM is detected. As mentioned above, IgM can be present from an old infection or early in seroconversion. A high avidity test result can indicate infection older than 14 to 16 weeks. A low avidity can occur in infection that is recent, within 14 to 16 weeks but also can remain positive for extended times (even years). So, with a positive IgM, an avidity test may be helpful, and should be utilized, to establish timing of infection.

Extremely rarely, a woman infected before conception has transmitted infection to her fetus with reports of this being associated with an unusual strain of Toxoplasma that is different from the initial infection. There is not presently a serologic test that can identify this rare occurrence. A hook study using the Abbott Architect by Mahinc et al [26] showed that the ICT performed well across a range of IU, with samples from <1 IU to much higher levels, detected IgM alone, IgG alone and combined IgG and IgM. The study of Chapey et al [25] showed a measured AUC close to 1, using quantitation with scanning. The ICT performed better than or as well as every predicate FDA cleared test using the FDA, CLIA cleared Remington Specialty Toxoplasma Serology PAMF Reference laboratory tests with the CDC set of approved samples. Summary of additional strengths. Strengths include: (1) evaluation of a screening test for Toxoplasma infection that detects G, Mor G plusMantibodies with high precision; (2) that meets WHO REASSURED criteria; and (3) creates a new algorithm for screening for Toxoplasma infection. This test identifies infection using whole blood or serum with NPV 100%, PPV 100%, AUC 1 in all studies in the US with thousands of sera and whole blood tests by multiple testers and participants in varied settings. Results with an earlier pink line (red bead) then black bead did not differ for serum samples. This work demonstrates that previously published pink line kit testing of sera and currently used black line kit testing of sera are comparable. This was documented with 1074 sera from 4 sites in the south of France, with similar findings of comparability in Chicago studies. This black bead kit then was used for testing whole blood. The sensitivity and specificity did not differ between serum and whole blood samples in the Chicago, USA studies. It lowers costs and improves acceptability of testing. Serial studies were designed and carried out to address regulations in a manner that meets FDA and CLIA criteria for clearance and waiver in US and CE mark in Europe. This is so this test can be used easily with confidence in its efficacy, safety and reliability. This test creates a novel screening model for testing pre-pregnant and pregnant persons, for rapid confirmatory testing in a clinical lab. Adverse consequences of infection can progress rapidly in utero making prompt diagnosis to enable treatment of value [3]. As discussed below, this test is also useful for those at risk for or who have retinal disease, lymphadenopathy, in epidemic settings, for public health assessment, for testing medicines or vaccines that are curative or prevent infection and disease, and other clinical circumstances enabling prompt diagnosis and treatment, as also discussed under “Implications for Public Health” below.

Limitations include thefollowing: This test does not distinguish IgG and IgM, whether IgM is part of new, acute acquisition, residual, persistent IgM from earlier infection pre-pregnancy or late in gestation, but can distinguish a false positive result from a commercial test that is slightly or more above the cut off value in the commercial predicate tests. In low prevalence areas false positives can confound care more frequently. The performance in the US and Paris is so strong that it is not possible to present a proportion of positives with 1 primary infection per 1000 pregnancies. Differences in the percentages of sero-positives, reflects differing participant populations, with some whole blood tests for large numbers of participants occurring in high prevalence settings of Morocco and Colombia, and in some US studies in US enriched for seropositive persons. The Chicago/Paris and Cincinnati studies with very low prevalence with larger numbers of sera also influence those proportions. The sensitivity and specificity of the serum and blood testing did not differ significantly in any study with simultaneous testing in the USA in multiple studies. Test methodology names to obtain links on Google are in the Box 2 legend.

Experience in Tunisia is cautionary, and it may be more problematic as there were more false positive results. In the future there might be more variability in different field settings. Positive results that influence patient care should be confirmed in Reference laboratory or with Western Blot for this reason. Thirty three of 39 French Reference laboratories use the Western blot as the gold standard confirmatory tests but this is not available currently in the USA. The ICT is intended to involve medical personnel and should include those who are skilled in interpretation of serologic results and patient care for those with Toxoplasma infection and toxoplasmosis. Even a technically perfect test for these diseases requires clinically knowledgeable users to properly provide informed medical care for an infection and illnesses with serious consequences. Proper care can be life, sight, cognition and motor function saving lifelong. This is not a test for lay users for these reasons. Testing before conception, to identify seropositive persons, and then testing regularly monthly through pregnancy for those who are IgG seronegative initially, would be ideal as it helps to obviate problems of persistent IgM, testing at the initial visit and often later times in gestation, anxiety provoking delays that can result in irreversible fetal damage, as well as false positive test results. Such damage in congenital toxoplasmosis, as well as in ocular toxoplasmosis can occur in very short times of less than a week, making diagnosis and initiation of treatment urgent and emergent. With clear seroconversion, treatment can be initiated presumptively while waiting for reference laboratory results. Positive results should always be confirmed. Minimizing the likelihood of false positive IgM while maintaining maximum sensitivity is a top priority for any point of care test candidate.

### Implications for Clinical Laboratory Practice

The ICT also should be very useful in clinical laboratories testing with sera with a potential false positive IgM result without IgG as described herein. It could function as a second-line test to confirm or find IgM specific for T. gondii is not present before sending the sample to a reference center, while continuing to follow the patient while awaiting Reference laboratory results. This is a major advance as this will save time and reduce the need for gold standard tests. It can help reduce concern for patients and physicians. When the ICT test is used initially with whole blood the only predicate test for confirmation needed will be if the whole blood test is positive. ICT not only helped to obviate the problems with false positives but also can result in detection of true positives and very early seroconversion as described herein and also recently acquired infections described elsewhere [9,23,24,26,27,30].

Implications for Public Health for Large Scale Gestational Screening Programs to reduce burden of congenital toxoplasmosis, to reduce the burden of eye disease from post-natal infections, and in a variety of other settings

We placed this work in the context of ongoing problems for healthcare and potential for direct and spillover benefit for the care of pregnant women and their families (14]). We also placed these studies 1 to 12 herein in a historical context building parts of a toolbox working toward a role of screening using WHO REASSURED criteria compatible tests in the elimination of congenital toxoplasmosis.

As considered above for individual clinical circumstances and in the local clinical laboratory there are similar additional public health considerations in the same categories. There also are a variety of other clinical and epidemiologic circumstances where knowing T. gondii serologic antibody status can be of considerable clinical and public health utility and benefit [1,2,6,7,27,32-9,46-50]. Very high-quality, low-cost screening tests such as the ICT can improve infectious diseases care in gestation, eliminating perinatal infections. There are a variety of public health threats to pregnant women from wild-type Toxoplasma as well as parasites that can be infected with single stranded RNA viruses in fresh and sea water, venison, worsening with heavy rainfall, endemic and epidemic. Value extends beyond care for toxoplasmosis to other aspects of health care for pregnant women and other clinical and research settings. Large scale screening programs reduce the burden of congenital toxoplasmosis for individual suffering and societal costs.

### Implications and Perspectives for Further Research

Congenital toxoplasmosis is a treatable and preventable disease, and physicians and other obstetrical providers now have the tools, in-hand, to improve outcomes and reduce patient and familial suffering. This screening, the standard of care in other countries, is now increasingly feasible in countries like the United States, where the primary argument against screening has been its economic burden. In the development of this test and other high-functioning point-of-care tests, there is potential for transformation in the provision of obstetrical care to improve maternal-child health. These benefits are amplified in subpopulation demographics in the USA [28] and regions of the world where the burden of disease is even higher. Examples of this occur in the Lancaster Amish population in the USA [8,12,32,46], parts of Florida, are likely in other US subpopulations [28], and occur in Central [36] and South America [47], and parts of Africa [29].

Use of the ICT for the Cincinnati maternal cohort study found ICT to be efficient (Study 9). Due to the small proportion who were seropositive, we were unable to test for any clustering by known risk factors for exposure: none of the individual socio-economic or location factors in a regression analysis achieved statistical significance. A larger overall sample size will be needed to evaluate risk factors in this population. Reasons for relatively low prevalence in Cincinnati in this cohort remain to be discovered. Even with the low prevalence found, it is likely still that gestational screening would be worthwhile.

Our recent study in Colombia also demonstrated high acceptability of a single use of the POC on a large scale of 783 women and 30 providers [47]. Although Toxoplasma infections occur in all demographics it was a particular problem in those who had lower education and socioeconomic status [31,47]. To understand risk factors during gestation and to develop programs to prevent such infection will require monthly screening in areas of high to low prevalence.

The implementation of this study in the clinical trial and the QI limit of detection study demonstrated that it should be easy to introduce this test into obstetrical or other practice taking little extra time or causing inconvenience. For example, when patients are evaluated for vital signs, blood pressure, glucose including by finger stick, by a medical assistant or nurse, the test can be performed and the cassette can also be brought to the obstetrician or other health care practitioner for additional reading and entry into the medical record. Photography, using a smart phone for documentation, could easily be included for when the care provider requires assistance with interpretation and for additional documentation for the patient and incorporation into the medical record.

In France screening was mandated by law. In Austria those screened received additional health care benefits. In Colombia it was introduced through practice societies. In the USA those in advisory positions recommended that education, easy feasibility, low cost would result in those who would benefit choosing to have testing incorporated in medical practice and USA patient culture at many levels by personal preference. The acceptability study demonstrated that informed patients would want this and obstetricians could use this comfortably and without inconvenience in their practice. It could easily be introduced into family practice and adolescent pediatric care to identify seropositive patients at risk of this most common retina disease due to infection and loss of sight. Such screening in adolescence could also provide pre-pregnancy testing for young women to allow knowledge of who is seronegative and should be screened during pregnancy. Pre-marital/conception screening, as initially occurred in France, could also be helpful as families plan to have children. As Toxoplasma has been transmitted by organ donation and white blood cell transfusion and by sperm in domestic nonhuman animals, can relapse with immune suppression, and may be causative for epilepsy and some neurodegeneration, there are a number of other medical settings where knowledge of Toxoplasma serologic status may be useful.

A future possibility to obviate the potential limitation of use of saliva which does not perform well with ICT includes a test that might be used with saliva or serum or whole blood in conjunction with the ICT if developed further in the future. This nanogold NIRMIDAS test was studied with saliva, serum, and whole blood. It was found to have high sensitivity and specificity and dye test precision for the detection of IgG, and for IgM [37]. We had suggested earlier this might be an ideal test to use before conception or if cost were constrained and it was feasible for initial testing in gestation. Although finger stick for glucose is standard, easy, and familiar in obstetrical practices, obtaining saliva may be viewed as less difficult than whole blood. Thus, some might view testing using saliva a potential advantage. However, the nanogold for any sample, so far, would require transport to where the machine is, associated delays to reach a clinical laboratory, and electricity and a sophisticated machine for testing. Recently manufacture of this nanogold test was discontinued. NTRMIDAS has also used a gold bead ICT for SARS CoV-2, but nothing like this has been produced for Toxoplasma to date. The diagnosis and management of Toxoplasma infection requires a knowledgeable health care provider urgently making home testing of saliva less advantageous.

Another consideration for the future includes the evaluation of the potential for cost savings to individuals, health care providers and systems as well as limiting suffering for individual patients and their families. As a foundation for such analyses in a variety of settings, to understand if the ICT should be widely used in gestational screening programs, studies of cost efficacy were performed [14]. Initially, we, with economists, created a mathematical model which can be used for such analyses in various settings. We first demonstrated potential cost benefit using health care data in the US [14], which found a financial cost saving incentive for screening programs sponsored by government health care systems as took place in France. With this first adaptable mathematical model [14], we applied it to actual costs in Austria [15] and then in France [16,17] with economists Drs. Stillwaggon and Sawers. Cost/benefit was shown elegantly by Stillwaggon, Sawers, Prusa, and Kasper to occur even with low prevalence as in Austria. Gestational screening was found to be 14-fold cost saving for the health care system, as well as being good for people, saving life, sight, cognition and motor function in these countries [16,17]. The important study of Mandelbrot et al [3] demonstrated the benefit for improved gestation outcomes from prompt diagnosis and treatment, as a major consideration for a screening program. A well-functioning point of care test meeting WHO REASSURED criteria, adds time and cost savings as shown by our analyses herein. In the future this could be an additional cost savings and benefit consideration for gestational screening and treatment as well as in a variety of health care systems and in epidemiologic study.

In future studies, and perhaps ultimately in the future in clinical practice, the ICT also might be useful to demonstrate efficacy of candidate anti-parasitic agents [51]. This could be useful in establishing definitive cure of infection. This would be by demonstrating elimination of presence of any serum antibody to T. gondii with elimination of the three clinical forms of T. gondii (tachyzoites, bradyzoites in cysts, and “persister” organisms). This might be used to establish definitive cure in those treated in clinical trials, and with the possible goal of ICT use, ultimately, in clinical practice in this manner. It might be useful to establish vaccine efficacy [52]. The work herein provides part of the foundation for such studies. The ICT has also been utilized in studies of seroprevalence in wild-life and in characterizing epidemics taking place in zoos in primates and marsupials [53,54]. These latter studies are being performed in conjunction with studies of a new vaccine for zoo animals. In the future the ICT could be an efficient, low cost tool for one health and other epidemiologic studies of prevalence, and in epidemic as well as endemic settings.

Implications of Additional Considerations, and Perspectives, Concerning this Novel Test, Paradigm, Implementation, and Practical Use of this Approach

The following information addresses some of these special considerations: For context, we emphasize that our initial specific overall objectives of this study were to determine the performance of the ICT which we found to be sensitive and specific as described herein. When we found high performance in initial studies, our objectives were to carry out a series of well-controlled studies to assess whether the test met WHO REASSURED criteria for a point of care test, could meet all FDA and CLIA testing requirements to enable its use in the United States and elsewhere, and to determine feasibility of implementation and acceptability of use of the ICT in multiple real-life clinical settings and in an epidemiologic study. These objectives were achieved successfully as outlined above. Nonetheless, even such a high performing test was not developed and does not function well in a vacuum. Rather, this is part of a tool box beginning with knowledge of the diseases Toxoplasma causes and their consequences, and thereby whether, how, when and where to use the test. Certain special considerations concerning ease of use, learning to use the test, who could use it successfully, feasibility, ease of implementation, reproducibility, repeatability, and acceptability became evident in our studies. Herein, in all our studies in Chicago, per IRB regulation, 100% of our results were confirmed, and in practical use any positive result was also confirmed. Reference laboratory back up must be performed for any positive ICT result during gestation, especially seroconversion. We do not advocate home testing by patients, but believe testing should be performed with availability of a well-educated, knowledgeable physicians'/care givers' advice because of the substantial impact of this congenital infection on outcomes lifelong.

The FDA/CLIA regulations required multiple settings and multiple testers in these separate stepwise studies. The variety of settings for point of care finger prick for whole blood, as shown in the individual patient/participant tables varied and the results were robust in all. Among all our US studies herein, testers included those with a wide variety of relevant backgrounds and a wide variety of relevant settings listed in Box 2 legend. All US tests were read by the testers and additional medical and laboratory personnel, and readings were documented by photograph. All readings in the US were congruent among multiple readers who worked independently and who did not influence each-others' readings of the test kit or photographs of the test kits. Readings were consistently congruent by varied testers in different settings. In the US, every person who had a whole blood reading had serum results. The earlier pink line serum test was found to be congruent with the black line whole blood and serum tests herein. Photographic documentation was performed with all US study readings in Chicago, and at Stanford earlier, with multiple congruent readings both for whole blood and for serum in Chicago.

As we did in these studies, we suggest that the results influencing clinical care, for example seroconversion or a positive test during gestation, be confirmed in a reference laboratory, and also be documented by smart phone or other photograph. Concerning education, the test kit brochure has a 24/7 telephone number where photos can be reviewed for assistance. We have created a You tube movie linked to reference [23], and an instructional test set of photos we will place on our website. We have created a suite of educational materials for various audiences (high schoolers, when considering pregnancy, during pregnancy, medical students, residents, fellows, practicing physicians, in text book chapters) [6,7]. Educational materials have improved knowledge in all those settings [32-5]. Concerning educational materials, families of affected children (some of whom are now adults) want to introduce information at multiple levels. This was to try to build a robust system of informed persons and call systems when physicians, other care givers, or patients need advice. In the US, in June 2023, eight states (Hawaii, Arkansas, Nebraska, Kansas, Kentucky, Pennsylvania, Minnesota, Wisconsin and Delaware) have made it the law to report toxoplasmosis. They are creating a knowledgeable cohort of CDC epidemiologists in those state programs. They have created uniform reporting criteria which were approved by the full Council of CDC epidemiologists nationally and in all 50 states and territories in June 2023. To help to enable screening in those settings as noted above, we have made educational materials freely available [32-5]. We view the ICT as part of a “toolbox” to implement high quality care to prevent toxoplasmosis, where education and expertise in interpretation and advice are critical. Tables 1, 2, 3A and 3B and Box 2 illustrate feasibility, and successful implementation of testing. Although ICT result informs initial approach to care, a positive result should be confirmed and in certain clinical circumstances such as during gestation with differentiation of acute from chronic infection is essential. Exceptions for need for confirmation include epidemiologic prevalence studies where the test is not influencing direct patient care. The high performance of the ICT and development of this new paradigm for care (as shown in FIG. 6 and Table 6) can help to improve outcomes for congenital toxoplasmosis substantially and contribute to its elimination. As noted above, this neglected disease causes a significant health burden with 190,000 infants or more infected each year causing 1.2 million disability adjusted life years. Obstetricians, nurse midwives, family practitioners, obstetrical nurses, and other obstetrical providers, neonatologists, pediatricians, infectious diseases specialists, policy makers, public health officials, and others are uniquely positioned to intervene to prevent this disease and improve the health of both mother and child. POC test based monthly gestational screening of seronegative patients for T. gondii infection provides a valuable tool in the obstetric armamentarium to ensure maternal-child wellness and lower its health-care burden around the world. Inclusion of POC-ICT into Toxoplasma diagnosis brings considerable promise.

TABLE 8
(original Table 6 in PNTD paper and in text of this specification below)
Table 6. Summary of Role of ICT and Novel Paradigm in Approach to Screening and Actions Taken.

| Reason for | | | |
|---|---|---|---|
| screening | Time of ICT | Back up Te [symbol] t | Enables diagnosis for Treatment |
| Pre-pregnancy | [symbol] | [symbol] if positive [symbol] | Know serology |
| Screen to | Considering | | Eye [symbol] positive |
| establish | pregnancy | | Pre-pregnancy |
| serologic states | | | |
| To [symbol] | [symbol] | [symbol] G | [symbol] clinical findings |
| [symbol] other | [symbol] | [symbol] G | Know serology |
| | | | Eye [symbol] positive |
| Pregnancy | Before | Positive [symbol] | Diagnosis [symbol] |
| | Weeks | [symbol] > | [symbol] acute |
| | [symbol] and | [symbol] can be used [symbol] | [symbol] > |
| | [symbol] if | positive [symbol] with whole [symbol] sample and in [symbol] negative | Treat [symbol] |
| | negative | [symbol] > [symbol] | transmission to fetus or [symbol] |
| | | Consider [symbol] | |
| | | Management is [symbol] | |

[symbol]

The use of [symbol] are not gold standard testing in [symbol] [symbol]
documented with [symbol] timing of [symbol] This [symbol] can be used [symbol]
Co [symbol] tests are [symbol] determine whether [symbol] to [symbol]
All negatives would [symbol] follow [symbol] up [symbol] while all [symbol] would [symbol] would [symbol] and analysis of [symbol] estimate the [symbol] The [symbol] replace [symbol] standard testing [symbol] and that at the point of care with a highly [symbol] testing takes place [symbol] [symbol]
indicates data missing or illegible when filed

This new paradigm is to have a test that meets WHO REASSURED standards available promptly at the time the test is performed and to have a first backup of positive results in serum rapidly in the local laboratory in the context of knowledgeable medical care and reference laboratory backup testing. Table 6 contains a summary of approaches to testing in screening programs and some of the ways in which results for this novel paradigm are actionable for care providers. This includes and is especially focused for the pregnant woman and her fetus. Those who are seronegative are tested monthly until 6 weeks post-partum. Pre-conception testing is obtained when possible. Testing for JgG and IgM is obtained for those who are seropositive, and for anyone who has IgM an avidity and AC/HS test is potentially helpful to date infection. The ICT is also potentially useful in a variety of other clinical settings. When such tests have undergone appropriate evaluation by the FDA and CLIA, as they have undergone in CE-mark evaluation and approval in Europe, this testing can enable substantial improvements in management of risks associated with exposure to T. gondii.

### CONCLUSIONS

This ICT meets WHO REASSURED criteria, with extremely high performance in testing serum and/or whole blood at the point of care to rapidly provide evidence about whether a person is infected or not. The studies herein also demonstrate effective initial use of the ICT for rapid backup of positive results in the local laboratory thereby providing considerable benefit in concert with knowledgeable medical care and reference laboratory backup testing.

### REFERENCES

1. McLeod R, Kieffer F, Sautter M, Hosten T, Pelloux H. Why prevent, diagnose and treat congenital toxoplasmosis?Mem Inst Oswaldo Cruz 2009; 104:320-44. https://doi.org/10.1590/s0074-02762009000200029 PMID: 19430661
2. McLeod R, Lykins J, Noble A G, Rabiah P, Swisher C, Heydemann P, et al Management of Congenital Toxoplasmosis. Curr Pediatr Rep 2014; 2:166-194
3. Mandelbrot L, Kieffer F, Sitta R, Laurichesse-Delmas H, Winer N, Mesnard L, et al. Prenatal therapy with pyrimethamine+sulfadiazine vs spiramycin to reduce placental transmission of toxoplasmosis: a multicenter, randomized trial. Am J Obstet Gynecol 2018; 219(4): 386.e1-386.e9. https://doi.org/10. 1016/j.ajog.2018.05.031 PMID: 29870736
4. Pappas G, Roussos N, Falagas M E. Toxoplasmosis snapshots: global status of Toxoplasma gondii seroprevalence and implications for pregnancy and congenital toxoplasmosis. Int J Parasitol 2009; 39:1385-1394. https://doi.org/10.1016/j.ijpara.2009.04.003 PMID: 19433092
5. Torgerson P R, Mastroiacovo P. The global burden of congenital toxoplasmosis: a systematic review. Bull World Health Organ 2013; 91:501-508. https://doi.org/10.2471/BLT.12.111732 PMID: 23825877
6. McLeod R, Van Tubbergen C, Boyer K M. Toxoplasmosis (Toxoplasma gondii). In: Kliegman R, St. Geme J, eds. Nelson Textbook of Pediatrics. 20th ed. Elsevier, 2016: 1723-1733. New edition in press 2024.
7. McLeod R, Cohen W, Dovgin S, Finkelstein L, Boyer K M. Human Toxoplasma Infection. In: Weiss L, Kim K, eds. Toxoplasma gondii. 3rd ed. Elsevier, 2020:117-228.
8. El Bissati K, Levigne P, Lykins J, Adlaoui E B, Barkat A, Berraho A, et al. Global initiative for congenital toxoplasmosis: an observational and international comparative clinical analysis. Emerg Microbes Infect 2018; 7:165. https://doi.org/10.1038/s41426-018-0164-4 PMID: 30262847
9. Wallon M, Peyron F, Cornu C, Vinault S, Abrahamowicz M, Kopp C B, et al. Congenital Toxoplasma Infection: Monthly Prenatal Screening Decreases Transmission Rate and Improves Clinical Outcome at Age 3 Years. Clin Infect Dis 2013; 56:1223-1231. https://doi.org/10.1093/cid/cit032 PMID: 23362291
10. Gomez-Marin J E, Montoya-de-Londono M T, Castano-Osorio J C. A maternal screening program for congenital toxoplasmosis in Quindio, Colombia and application of mathematical models to estimate incidences using age-stratified data. Am J Trop Med Hyg. 1997; 57(2):180-6. https://doi.org/10.4269/ajtmh.1997.57.180 PMID: 9288813.
11. Carneiro A C, Machado A S, Be\acute{ }la S R, Costa J G, Andrade G M, Vasconcelos-Santos D V, et al. Reply to Gomez Man\hat{ }n. J Infect Dis 2016; 214(4):656. https://doi.org/10.1093/infdis/jiw211 PMID: 27357342.
12. McLeod R, Boyer K M, Lee D, Mui E, Wroblewski K, Karrison T, et al. Prematurity and severity are associated with Toxoplasma gondii alleles (NCCCTS, 1981-2009). Clin Infect Dis. 2012; 54(11):1595-605. https://doi.org/10.1093/cid/cis258 PMID: 22499837
13. Peyron F, McLeod R, Ajzenberg D, Contopoulos-Ioannidis D G, Kieffer F, Mandelbrot L, et al. Congenital Toxoplasmosis in France and the United States: One Parasite, Two Diverging Approaches. PLoS Negl Trop Dis 2017; 11:e0005222. https://doi.org/10.1371/journal.pntd.0005222 PMID: 28207736
14. Stillwaggon E, Carrier C S, Sautter M, McLeod R. Maternal Serologic Screening to Prevent Congenital Toxoplasmosis: A Decision-Analytic Economic Model. PLoS Negl Trop Dis 2011; 5:e1333. https://doi. org/10.1371/journal.pntd.0001333 PMID: 21980546
15. Prusa A-R, Kasper D C, Sawers L, Walter E, Hayde M, Stillwaggon E. Congenital toxoplasmosis in Austria: Prenatal screening for prevention is cost-saving. PLoS Negl Trop Dis. 2017; 11:e0005648. https://doi.org/10.1371/journal.pntd.0005648 PMID: 28692640
16. Binquet C, Lejeune C, Seror V, Peyron F, Bertaux A C, Scemama O, et al. The cost-effectiveness of neonatal versus prenatal screening for congenital toxoplasmosis. PLoS One. 2019; 14(9):e0221709. https://doi.org/10.1371/journal.pone.0221709 PMID: 31532766
17. Sawers L, Wallon M, Mandelbrot L, Villena I, Stillwaggon E, Kieffer F. Prevention of congenital toxoplasmosis in France using prenatal screening: A decision-analytic economic model. PLoS One. 2022 Nov. 4; 17(11):e0273781. https://doi.org/10.1371/journal.pone.0273781 PMID: 36331943; PMCID: PMC9635746.
18. Maldonado Y A, Read J D, Committee on Infectious Diseases. Diagnosis, treatment, and prevention of congenital toxoplasmosis in the United States. Pediatrics 2017; 139(2):e20163860. https://doi.org/10. 1542/peds.2016-3860 PMID: 28138010
19. Kosack C S, Page A L, Klatser P R. A guide to aid the selection of diagnostic tests. Bull World Health Organ. 2017; 95(9):639-45. https://doi.org/10.2471/BLT.16.187468 PMID: 28867844
20. Liesenfeld O, Press C, Montoya J G, Gill R, Isaac-Renton J L, Hedman K, et al. False-positive results in immunoglobulin M (IgM) Toxoplasma antibody tests and importance of confirmatory testing: the Platelia Toxo IgM test. J Clin Microbiol. 1997 January; 35(1):174-8. https://doi.org/10.1128/jcm.35.1.174-178.1997 PMID 8968902; PMCTD: PMC229533.
21. Garry D J, Elimian A, Wiencek V, Baker D A. Commercial laboratory IgM testing for Toxoplasma gondii in pregnancy: a 20-year experience. Infect Dis Obstet Gynecol. 2005 September; 13(3):151-3. https://doi.org/10.1080/10647440500148024 PMID: 16126500; PMCID: PMC1784571.
22. Dhakal R, Gajurel K, Pomares C, Talucod J, Press C, Montoya J G. Significance of a Positive Toxoplasma Immunoglobulin M Test Result in the United States. J Clin Microbiol 2015; 53(11):3601-05. https://doi.org/10.1128/JCM.01663-15 PMID: 26354818
23. Lykins J D, Li X, Levigne P, Zhou Y, El Bissati K, Clouser F, et al. Rapid, inexpensive, fingerstick, wholeblood, sensitive, specific, point-of-care test for anti-Toxoplasma antibodies. PLoS Negl Trop Dis 2018; 12:e0006536. https://doi.org/10.1371/journal.pntd.0006536 PMID: 30114251
24. Begeman I J, Lykins J, Zhou Y, Lai B S, Levigne P, El Bissati K, et al. Point-of-care testing for Toxoplasma gondii IgG/IgM using Toxoplasma ICT IgG-IgM test with sera from the United States and implications for developing countries. PLoS Negl Trop Dis 2017; 11:e0005670. https://doi.org/10.1371/journal.pntd.0005670 PMID: 28650970
25. Chapey E, Wallon M, Peyron F. Evaluation of the LDBIO point of care test for the combined detection of toxoplasmic IgG and IgM. Clin Chim Acta 2017; 464:200-201. https://doi.org/10.1016/j.cca.2016.10.023 PMID: 27765564
26. Mahinc C, Flori P, Delaunay E, Guillerme C, Charaoui S, Raberin H, et al. Evaluation of a New Immunochromatography Technology Test (LDBio Diagnostics) To Detect Toxoplasma IgG and IgM: Comparison with the Routine Architect Technique. J Clin Microbiol 2017; 55:3395-3404. https://doi.org/10.1128/JCM.01106-17 PMID: 28954897
27. Gomez C A, Budvytyte L N, Press C, Zhou L, McLeod R, Maldonado Y, et al. Evaluation of Three Pointof-Care Tests for Detection of Toxoplasma Immunoglobulin IgG and IgM in the United States: Proof of Concept and Challenges. Open For Inf Dis 2018; 5:ofy215. https://doi.org/10.1093/ofid/ofy215 PMID: 30393749
28. Lykins J, Wang K, Wheeler K, Clouser F, Dixon A, El Bissati K, et al. Understanding Toxoplasmosis in the United States Through “Large Data” Analyses. Clin Inf Dis 2016; 63(4): 468-75. https://doi.org/10. 1093/cid/ciw356 PMID: 27353665
29. El Mansouri B, Amarir F, Peyron F, Adlaoui E B, Piarroux R, El Abassi M, et al. Performance of a novel point-of- care blood test for Toxoplasma infection in women from diverse regions of Morocco. Microbes Infect 2021 December; 10(1):1675-1682. https://doi.org/10.1080/22221751.2021.1948359 PMID: 34165384
30. Abraham S, Piarroux R. Zhou Y, Tesic V, Abeleda A, Houhou-Fidouh N, et al European Journal of Clinical Microbiology & Infectious Diseases: Official Publication of the European Society of Clinical Microbiology, 26 Sep. 2023, 42(11):1327-1335 https://doi.org/10.1007/sI0096-023-04669-8, PMID: 37749274
31. Ben-Abdallah R, Kalboussi Y, Bellali H, Issaoui N, Souissi O, Maatoug R et al., Contribution of the Toxoplasma ICT IgG IgM test in determining the immune status of pregnant women against toxoplasmosis. J Laboratory Clinical Analysis. 35, 2021 e23749 https://doi.org/10.1002/jcla 23749 2021
32. Felin M S. Wang K. Moreira A. Grose A. Leahy K. Zhou Y, et al Building Programs to Eradicate Toxoplasmosis Part I: Introduction and Overview. Curr Pediatr Rep. 2022; 10(3):57-92. https://doi.org/10.1007/s40124-022-00269-w Epub 2022 Aug. 22. PMID: 36034212; PMCID: PMC9395898.
33. Felin M S. Wang K, Moreira A, Grose3 A. Leahy K. Zhou Y, et al. Building Programs to Eradicate Toxoplasmosis Part II: Education. Curr Pediatr Rep. 2022 September; 10(3):93-108. https://doi.org/10.1007/s40124-022-00267-y Epub 2022 Aug. 1. PMID: 36969368; PMCID: PMC10035399
34. Felin M S. Wang K. Raggi C, Moreira A, Pandey A, Grose A et al. Building Programs to Eradicate Toxoplasmosis Part III: Epidemiology and Risk Factors. Curr Pediatr Rep. 2022 September; 10(3):109-124. https://doi.org/10.1007/s40124-022-00265-0 Epub 2022 Jun. 22. PMID: 37744780; PMCID: PMC1051631
35. Felin M S. Wang K. Moreira A. Grose A. Leahy K. Zhou Y, et al Building Programs to Eradicate Toxoplasmosis Part IV: Understanding and Development of Public Health Strategies and Advances “Take a Village”. Curr Pediatr Rep. 2022; 10(3):125-154. https://doi. org/10.1007/s40124-022-00268-x Epub 2022 Aug. 16. PMID: 35991908; PMCID: PMC9379243
36. Flores C, Villalobos-Cerrud D, Borace J, Mcleod R, Fa\hat{ }brega L, NoreroX, et al. Prevalence and risk factors associated with T. gondii infection in pregnant women and newborns from Panama Pathogens. 2021 Jun. 17; 10(6):764.
37. Pomares C, Zhang B, Arulkumar S, Gonfrier G, Marty P, Zhao S, et al. Validation of IgG, IgM multiplex plasmonic gold platform in French clinical cohorts for the serodiagnosis and follow-up of Toxoplasma gondii infection.Diagn Microbiol Infect Dis. 2017 March; 87(3):213-218. https://doi.org/10.1016/j.diagmicrobio.2016.09.001 Epub 2016 Sep. 8. PMID: 28040304
38. Public Health Service, Department of Health and Human Services (US), Food and Drug Administration. (1997). FDA public health advisory: limitations of toxoplasmosis IgM commercial test kits (letter) Washington Department of Health and Human Services (US)
39. McLeod R., Sautter M, Rooney T, Morel J, Taub L, Taub D, et al Submission to the Committee on Public Health Illinois State Senate. Regarding: Prenatal and Neonatal Congenital Toxoplasmosis Prevention and Treatment Act, SB3667 in the context of the National Collaborative Chicago Based Congenital Toxoplasmosis Study. April 13. 2010.
40. Franck J, Garin Y J-F, Dumon H. LDBio-Toxo II Immunoglobulin G Western Blot Confirmatory Test for Anti-T Toxoplasma Antibody Detection. J Clin Microbiol. 2008 Jul. 1; 46(7):2334-8.
41. Maudry A, Chene G, Chatelain R, Patural H, Bellete B, Tisseur B, et al. Bicentric Evaluation of Six Anti Toxoplasma Immunoglobulin G (IgG) Automated Immunoassays and Comparison to the Toxo II IgG Western Blot. Clin Vaccine Immunol. 2009 Sep. 1; 16(9):1322-6. PMID: 19587151
42. Jost C, Touafek F, Fekkar A, Courtin R, Ribeiro M, Mazier D, et al. Utility of immunoblotting for early diagnosis of t. oxoplasmosis seroconversion in pregnant women. Clin Vaccine Immunol CVI. 2011 November; 18(11):1908-12.
43. Villard O, Cimon B, L'Ollivier C, Fricker-Hidalgo H, Godineau N, Houze S, et al. Serological diagnosis of Toxoplasma gondii infection: Recommendations from the French National Reference Center for Toxoplasmosis. Diagn Microbiol Infect Dis. 2016 January; 84(1):22-33.2015.09.009 PMID: 26458281
44. Douet T, Armengol C, Charpentier E, Chauvin P, Cassaing S, Iriart X, et al. Performance of seven commercial automated assays for the detection of low levels of anti-Toxoplasma IgG in French immune compromised patients. Parasite. 2019; 26:51.
45. Simon L, Fillaux J, Guigon A, Lavergne R-A, Villard 0, Villena I, et al. Serological diagnosis of Toxoplasma gondii: analysis of false-positive IgG results and implications. Parasite Paris Fr. 2020; 27:7.
46. Boyer K, Hill D, Mui E, Wroblewski K, Karrison T, Dubey J P, et al. Unrecognized Ingestion of Toxoplasma gondii Oocysts Leads to Congenital Toxoplasmosis and Causes Epidemics in North America Clin Infect Dis. 2011 Dec. 1; 53(11): 1081-1089. Published online 2011 Oct. 21. cid/cir667; PMCID: PMC3246875 PMID: 22021924
47. Londono-Martinez J C, Velasco-Velasquez S, Cordero-Lopez S, Osorio M F, Celis-Giraldo D, Thibodeau J, Baird I, McLeod R, Gomez-Marin J. Evaluation of the acceptability of point of care diagnostic test for prenatal toxoplasmosis (translational research phase III). J Infect Public Health. 2022 Nov. 23; 16 (1):15-24. PMID: 36446203
48. Mejia-Oquendo M, Marulanda-Ibarra E, Gomez-Marin J E, Mejia-Oquendo M, Marulanda-Ibarra E, Gomez-Marin J E. Evaluation of the impact of the first evidence-based guidelines for congenital toxoplasmosis in Armenia (Quindi\acute{ } o) Colombia: An observational retrospective analysis-NC-ND license (http://creativecommons.org/licenses/by-nc-nd/4.0/) 2021. https://doi.org/10.1016/j.lana. 2021.100010
49. Plazas M I, Mari\acute{ } n J S, Torres E, Londono J C, Celis-Giraldo D, Man\acute{ } n J E G. Frequency of natural antibodies and concordance analysis for anti-TOXOPLASMA IgM tests in Colombian sera of pregnant women. Diagnostic Microbiology and Infectious Disease 2022; 103:115733. 2022.115733 PMID: 35714429
50. Torres-Morales E, Go\acute{ } mez-Mari\acute{ } n J E. Evaluating a Toxoplasma IgG avidity ELISA test for diagnostic purposes during pregnancy and correlating it with Toxoplasma IgM and IgA in the Biomedical Research Centre's laboratory at the Universidad del Quindi\acute{ } o, 2008. Revista Colombiana de Obstetricia y Ginecologia 2008; 59.
51. McPhillie M J, Zhou Y, Hickman M R, Gordon J A, Weber C R, Li Q, et al. Potent Tetrahydroquinolone Eliminates Apicomplexan Parasites. Front Cell Infect Microbiol. 2020 Jun. 9; 10:203. PMID: 32626661; PMCID: PMC7311950.
52. El Bissati K, Zhou Y, Paulillo S M, Raman S K, Karch C P, Reed S, et al. Engineering and characterization of a novel Self Assembling Protein for Toxoplasma peptide vaccine in HLA-A*11:01, HLA-A*02:01 and HLA-B*07:02 transgenic mice. Sci Rep. 2020 Oct. 12; 10(1):16984. PMID: 33046728; PMCID: PMC7552409.
53. Strules J, Dawant T, Riese K, Gerhold R, Brown J, Olfenbuttel C, et al. Use of point of care test to determine the prevalence of antibodies to Toxoplasma gondii in black bears from North Carolina and Pennsylvania. J Parasitol. 2023 May 1; 109(3):221-224. https://doi.org/10.1645/22-72 PMID: 37327396.
54. Fasquelle F, Scuotto A, Vreulx A-C, Petit T, Charpentier T, Betbeder D. Nasal vaccination of six squirrel monkeys (Saimiri sciureus): Improved immunization protocol against Toxoplasma gondii with a nanoparticle-born vaccine. International Journal for Parasitology: Parasites and Wildlife International Journal for Parasitology: Parasites and Wildlife. 22, 2023: 69-74. PMID:
   Lessons in Toxoplasmosis Learned and Re-Learned in the USA and in a Global Initiative, 1974-Present that the ICT can Help Enable, Provide Solutions for and Benefit and Optimize Health Care

### Examples

In the USA currently, as during the past 50 years, there have been remarkable recent discoveries and advances, and new paradigms to treat to prevent adverse sequelae, for those with toxoplasmosis. Nonetheless, care for many people with toxoplasmosis is/has been complicated by delays in diagnosis, difficulties in initiating treatment, and errors in management when care could be/could have been optimized. Improvements in understanding and knowledge, systematic screening in gestation and an easier, less costly infrastructure to facilitate optimized care would prevent suffering, save lives, sight, cognition, and motor function. One of the explanations posited for why this disease is still neglected, misdiagnosed, with difficulties in optimizing management is the misconception that this infection of 2.5 billion people worldwide is so rare in the USA that improving care here in the USA is not worthwhile. We and others learn frequently about the potentially preventable suffering associated with these infections. This experience includes thousands of patients and their families. Herein, we developed a snapshot of prevalence of this infection in women of childbearing age from areas where we have received requests for consultations and referrals. Samples obtained for rubella testing were studied as this testing for antibodies to rubella virus usually is performed to establish rubella immunity in pregnancy. Other collections of sera also were used if their source from women of childbearing age could be identified. We found prevalence of Toxoplasma infections ranging from <2% to 50% across a mixture of payors in a number of sites across the USA, with median prevalence in these areas 15 to 22%. A lateral chromatographic immunodiffusion test that detects IgG and/or IgM specific for Toxoplasma, with very high sensitivity and specificity, was utilized for most determinations with additional tests confirming its high sensitivity, specificity, identifying congruence in samples of whole blood, plasma, serum, reproducibility, and ease of instruction for use. Examples of this powerful approach are summarized, illustrated, herein to identify and emphasize circumstances where care could be optimized and means whereby future outcomes could be markedly improved for patients and their families in the USA. Potential solutions for the problems we often still encounter in the USA are presented.

Prompt, optimized diagnosis, treatment, and care for toxoplasmosis can prevent, eliminate, or ameliorate loss of life, sight, cognition and motor function (1-110). Nonetheless, in the USA, there still are often delays, use of faulty and expensive commercially available diagnostic tests, improper use of medicines, unavailabile necessary medication and only at high cost, complications with health insurance coverage making proper care difficult, and widespread lack of understanding of by physicians of how to properly use available medicines to treat afflicted patients. There are critical lessons about ways to optimize care that can remedy some of these problems. Examples are illustrated herein by brief or more detailed case summaries and solutions are proposed.

### Materials and Methods

1974 to 1981. A collaboration between French and USA physician scientists, information learned during the AIDS epidemic, a series of French and German studies and basic laboratory work (11-14) led to further understanding of diagnosis and treatment of toxoplasmosis.

National Collaborative Chicago-based Congenital Toxoplasmosis Study (NCCCTS), 1981 to present. In 1981 an infant was referred to one of the authors, RMc. Review of the available literature led to phase 1, 2 studies, including randomized clinical trials defining treatments and outcomes in the USA and in France (15-25). This has resulted in definition of algorithms to optimize care. Health care funding, arbitrary pricing and availability of testing and medicines have complicated implementation of optimal care in the USA (26, 27) with recent advances providing examples of potential solutions to these problems (27-30).

Snapshot of prevalence of Toxoplasma infection for women of childbearing age. Anonymized serum samples were requested from clinical laboratories in areas of the USA from which participants in the NCCCTS were referred. This involved physicians who had cared for patients with toxoplasmosis, many with interest in better understanding how serologic screening with prompt treatment in gestation might improve outcomes. For some this included samples that had been collected for testing for antibodies to rubella. Prospective testing with informed consent also was conducted across a number of demographics. Payor mix for the laboratories was established

Consultations without charge connected to clinical care, direct referrals for care, and experience in the NCCCTS. These experiences formed the basis for the selection of cases illustrative of circumstances where optimization of care would have improved outcomes as well as examples where optimal care provide many examples of positive outcomes.

Development of solutions 1974 to the present including current status that is still evolving. Contrasts of these experiences have demonstrated ways in which speedy diagnosis, treatment and optimized care can result in favorable outcomes.

Morocco, 2025. Matrix, hook. Reproducibility, feasibility analyses were performed in some further examples wherein there was perfect correlation in matrix analysis of finger prick blood sample, serum, plasma for 50 pregnant women, perfect reproducibility study according to FDA guidelines, perfect hook study with black line similar to Mahinc pink bead with serum, compliance with return visits in study of 250 women needs improvement, information about Toxoplasma and diabetes risks is motivating for return visits. Usefulness in testing infants in NICU being tested combined with differential western blots with maternal and neonatal blood samples.

A recent study performed in Morrocco in 2025 demonstrates and validates the Toxoplasma ICT IgG-IgM test for the following:
i. The equivalence of sample types: serum, heparinized plasma, and heparinized whole blood
ii. The equivalence of results based on the collection method: fingerstick vs drawing blood
iii. the reproducibility across different test cassette batches
iv. The ease of use and interpretation of the test.

### Further Examples

List of problems to be solved in the USA. Table 1 includes a list of problems associated with human toxoplasmosis in the USA currently.

Problems to be solved A. Overview. B. specific problems in optimizing diagnosis, prevention and care. And C. unique metadata and new studies performed for additional new FDA requirements

A. Problems that can be addressed
Toxoplasma infects the brain of 2 billion people, lifelong, making It one of the most common parasitic infections in the world. (A billion brain parasite)
Chronic infection is incurable. It can reactivate at any time.
When acquired by an unborn baby or someone with a weakened immune system, this parasite destroys lives, sight, thought, and movement. And it can cause seizures. (Markedly different if untreated/treated; Rare US pediatric disease and Neglected Tropical Disease;
Bioterrorism pathogen; Worsening with global warming; Some isolates carry RNA virus with lethal pandemic potential )
Evidence that infection can associate with/cause/contribute to neurodegenerative and other diseases in some persons
May cause gestational diabetes
Causes suffering and is costly for care and society.

ICT can contribute to a novel paradigm solving each of these problems and others illustrated by the representative case presentations, such as: (1) False narratives that Toxoplasma seroprevalence is low throughout the US and education of avoiding risky behaviors, like information to discourage changing the litterbox of cats kept indoors and avoiding raw meat alone can protect against acute acquired infection. (2) Screening in gestation would not be cost effective (it is, e.g 14 fold cost savings in Austria with low seroprevalence and cost beneficial in France and feasible and valued by patients, families, obstetricians and maternal fetal medicine specimens when reasonable costs and easy availability occur. (3) Screening before conception and monthly during gestation optimizes diagnosis, prevention and treatment but irregular and less frequent screening is performed, if any screening takes place. (4) Delays in diagnosis and treatment and false positive IgM commercial tests harm the fetus and their families. (5) Prompt diagnosis, treatment, readily available medicines and knowledgeable care improve outcomes and are needed nationwide in the USA and worldwide. (6) Currently available diagnostic test for Toxoplasma IgM may result in false positive tests.

Included herein is information about data obtained from testing unique samples and persons with considerable metadata demonstrating utility of ICT in many demographics, settings and how it can solve practical problems efficiently.

### Example: EACH AND ALL of the Following, as Delineated in (a) to (p), can be Achieved in Various Embodiments Provided Herein

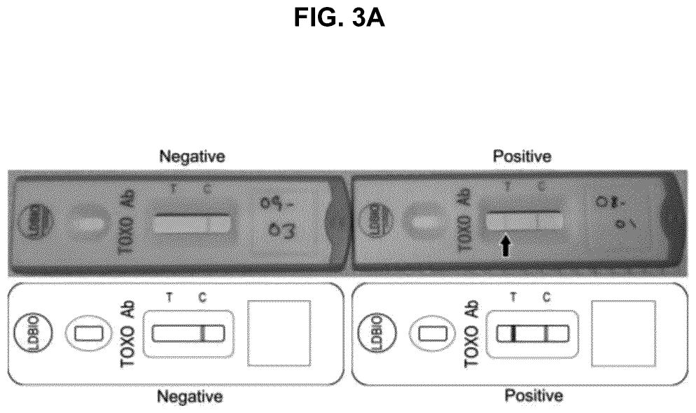

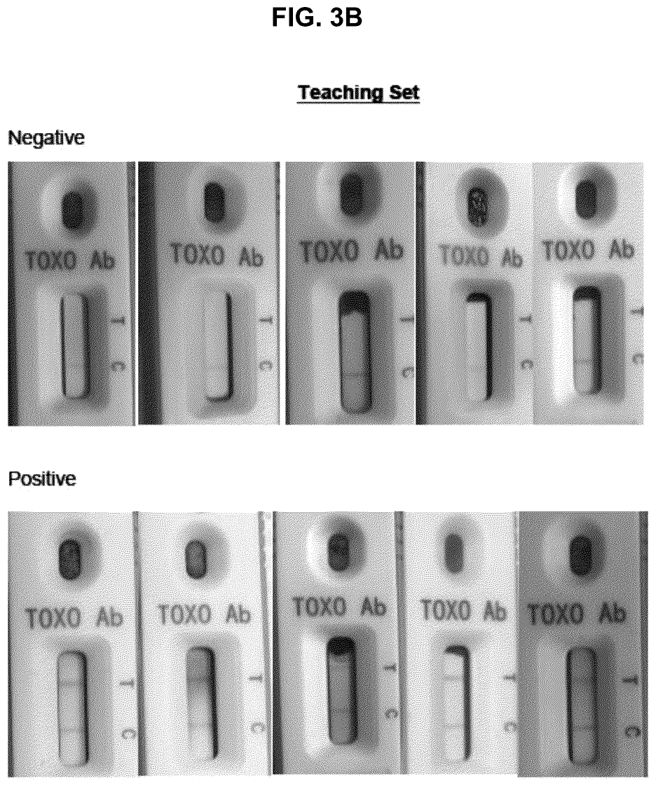

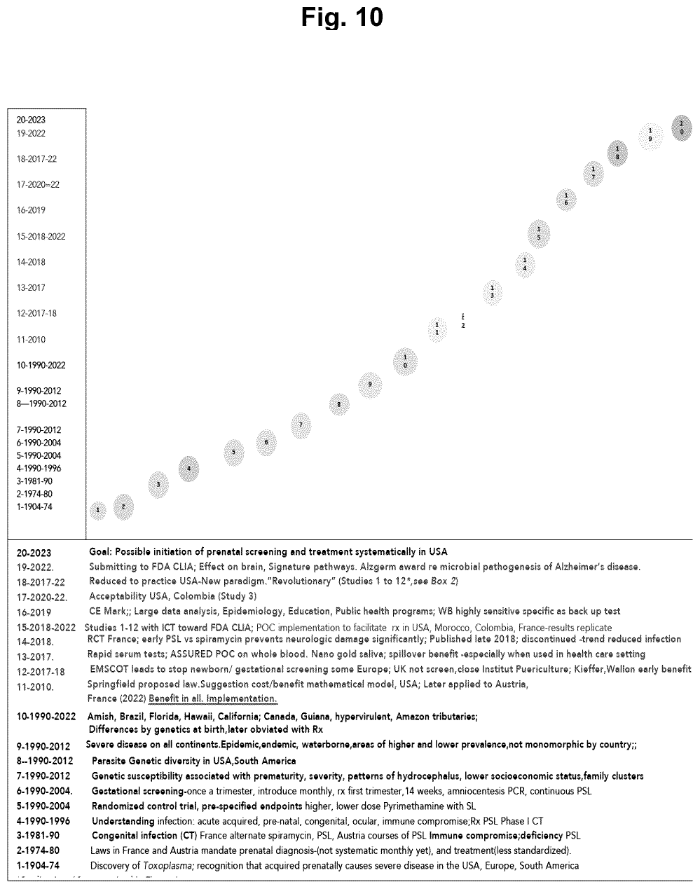

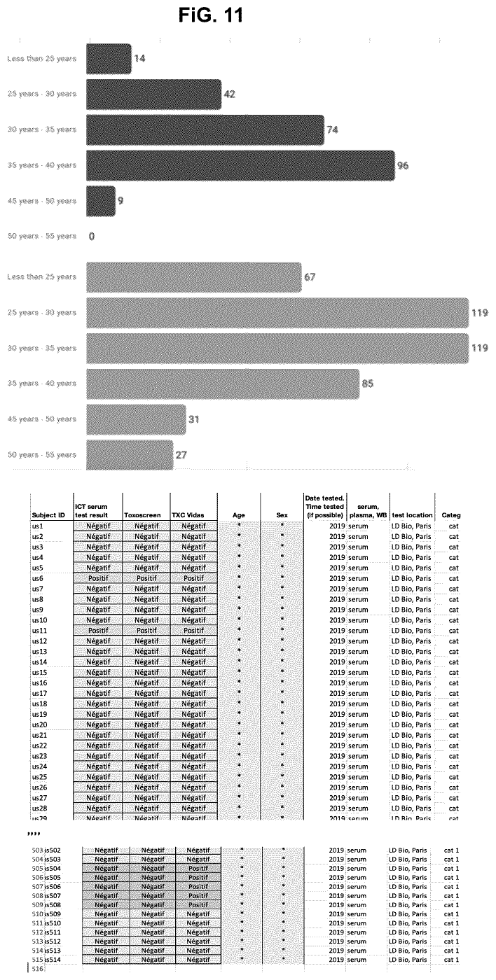

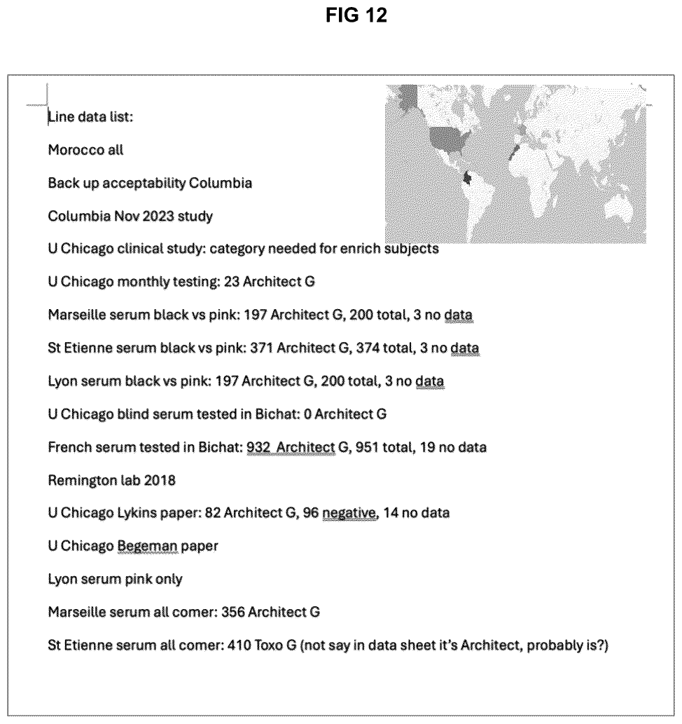

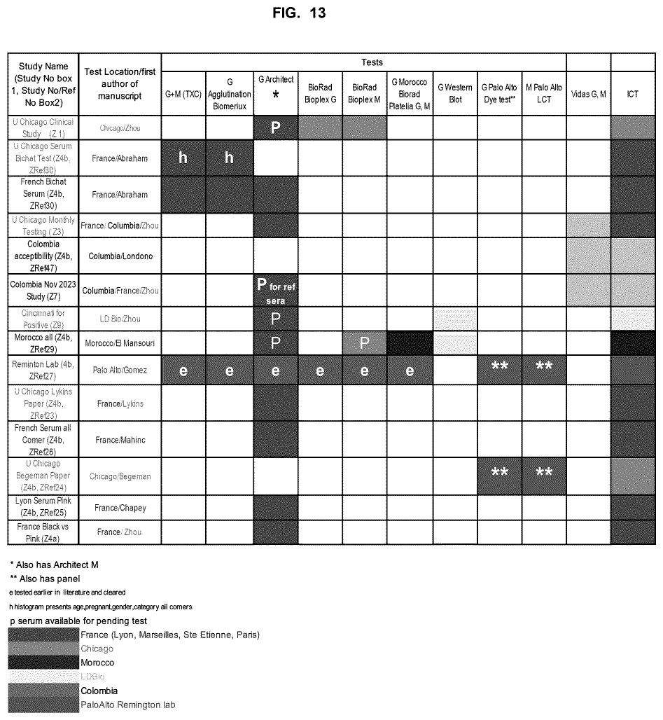

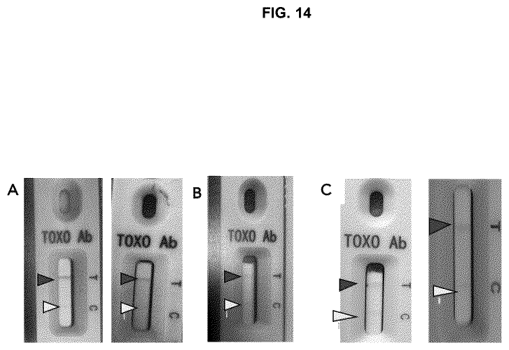

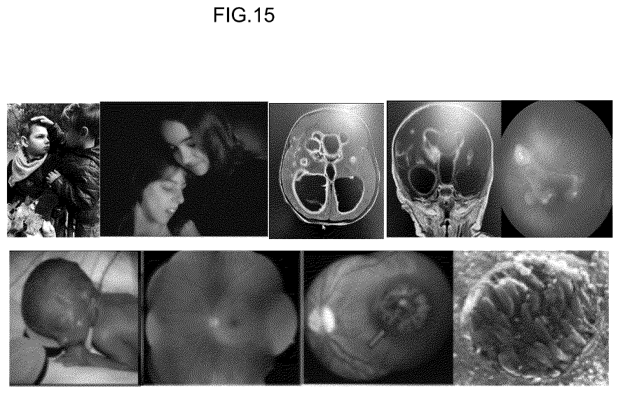

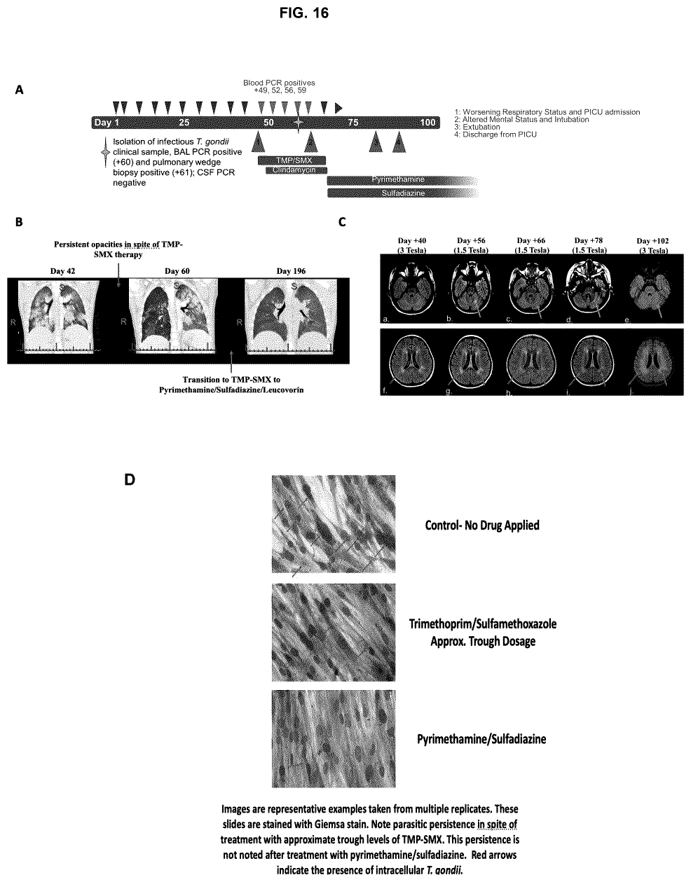

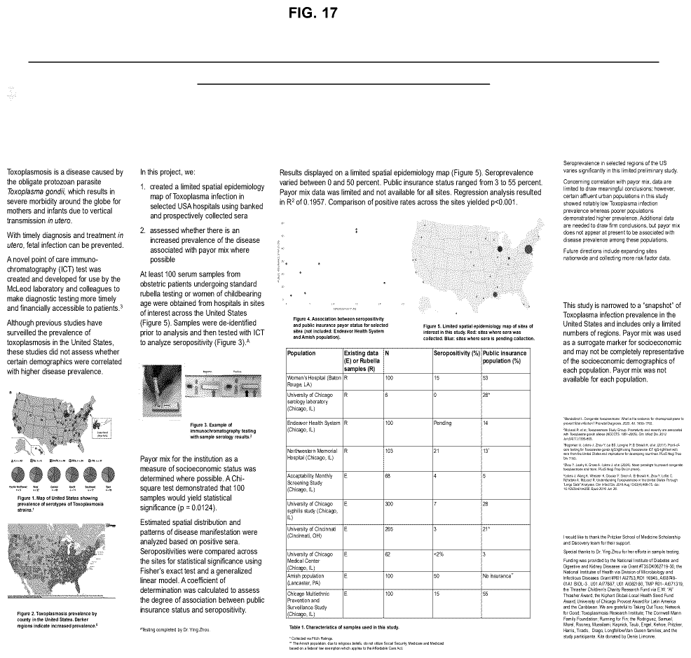

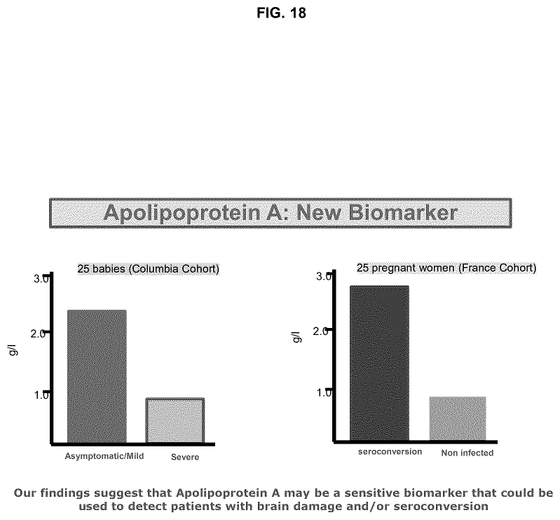

(a) The test provided herein can be a point of care or clinical laboratory test effective and can be used for testing whole blood collected by finger prick and capillary tube or by venipuncture, or serum, or plasma, and can, in various embodiments, be based on the principle of a black bead linked to Toxoplasma antigens made from in vivo cultures and a line of the antigen placed on the immunochromatography paper, a control line of antibody to antibody placed inferior to the T (test line of Toxoplasma antigen) on the lateral immunochromatography diffusion paper to demonstrate that antibody is present in the sample when it gives a blue color band in a position marked C (for control) on the plastic cassette when such antibody is present in the sample being tested, and thereby recognizes a sample that contains pentavalent IgM and/or bivalent IgG specific to Toxoplasma and wherein (1) these elements are present in a sample being tested and (2) when around 20 microliters of sample, (3) 4 drops of buffer eluent provided with the test kit are (4) added to a well near the top of the plastic cassette containing the lateral immunochromatography, immunodiffusion filter paper system as described above and (5) the reactions created by the connection of the constituents in the reaction that occurs are observed at the line of antigen marked with a T on the plastic cassette when viewed 20 to 30 minutes after placement of sample into well of the cassette and (6) a conclusion can be correctly reached that the sample contains antibody to Toxoplasma gondii of the IgM and/or the IgG isotype, as shown in (FIGS. 1 to 13, Tables 1 to 7 and showing detail of Table 3 in Tables 3A,B,C below. The development of need for a test like the ICT and then the ICT over time including the any and all use and benefits of the ICT and (7) that is specific to the single celled obligate intracellular, apicomplexan parasite named Toxoplasma gondii of any species, genetic type demonstrated to date to be present in North, Central or South America, Hawaii, the Caribbean islands, France, eastern Europe, northern Africa, New Zealand and the Philippines and (8) that the person whose sample being tested is capable of making antibody as indicated by the control blue band visible when viewing by the human eye and indicating the test kit is functioning properly and (9) that can be recorded and demonstrated by smart phone photography or other camera to document the result and (10) can be used to confirm test result interpretation with persons skilled or not specifically trained persons educated by reading the associated package instructions and (11) wherein all of these elements of this invention are present and they create a black line visible to the human eye on the filter paper at the T line, the conclusion is that the test is positive and antibody is present or no such black line is present and no antibody to this parasite can be detected and (13) expert advice should be obtained in interpreting the medical implications of the test result when it is used for patient care and (14) expert advice concerning use of this test and its interpretation can be obtained from the manufacturer LDBIO or others highly skilled and experienced in its use and (15) that there is documentation that this test is currently CEMarked as approved in Europe based on studies in France, the USA, North, Central and South America, and associated islands and North Africa and (16) a file has been prepared for submission to the dual 510K clearance and CLIA waiver and legal marketing in the USA FIGS. 11-13) and (17) that there is interest in co-marketing of this test to obviate problems with false positive IgM commercially available test results documented to occur with other currently marketed commercially available tests in the USA and France, where this test has been demonstrated to obviate this false positive result problem when sera are tested (18) This is a test that is proven to be capable of correctly recognizing infection of persons throughout the past half century in the USA. This is in clinical practice, the National Collaborative Toxoplasmosis Study, and other USA persons infected during that time. Infection was proven by other gold standard tests such as the Sabin Feldman Dye test and (19) A person known to be infected with the RH strain of Toxoplasma for in a laboratory accident 5 decades earlier, an isolate which has been sequenced (FIG. 14) and (20) persons identified by serotyping to be infected with serotypes I/III, II, atypical wild life type parasites including in the continental USA and islands where isolates differ (FIG. 15) and (21) proven for person from multiple regions of the USA and differing demographics and (22) tested with samples from the Centers for Disease Control FDA required testing set with perfect performance (23) more accurately than other ICT products that were evaluated by testing samples from persons residing in France and the USA (24) tested with whole blood, serum, and plasma samples in a matrix study (in Morocco) and (25) can be used to determine if and when antibody to Toxoplasma appears in the whole blood of a child with a stem cell transplant whose parasite can be sequenced from the same whole blood sequence establishing genotype and virulence factors present (FIG. 16) and (26) has no interference causing false positive test results in any of the following infections or illnesses: HIV, hepatitis A, B, C, EBV, malaria, influenza, gonococcal infection, syphilis, chlamydia, leishmania various immune compromised states and (27) has extensive meta data and line data for thousands of samples as required by the FDA (FIGS. 1 to 13, Boxes 1, 2, above) and (29) with all these metadata meets the myriad of requirements and new requirements of the CE mark process in Europe and the FDA so that it meets requirements for safe and effective use in the USA FIGS. 1 to 13, Boxes 1, 2,above) and (30) has been tested in an officially registered study with Clinical Trials.gov ) NCT04474132, IRB20-0442, Point of Care Testing for Presence of Current and or Previous T. gondii Infection) in accordance with all requirements for such a study and (31) is being used in a study of prevalence in the USA (FIG. 17) and of cognitive impairment, Alzheimer's disease and superagers and why chronic Toxoplasma infection is associated with gestational diabetes, fetal loss, still birth, prematurity, and can be implemented in clinical settings, in family, maternal fetal medicine, obstetric, in vitro fertilization, pediatric, internal medicine, geriatric, retina, ophthalmology, pediatric ophthalmology, stem cell and other transplant, infectious diseases, neurology, neurosurgery, neonatal intensive care unit, and other practices. Complemented by the LDBIO Western Blots to compare mothers and infants in appropriate clinical settings, and (32) with all these metadata brings more understanding of its utility in multiple clinical settings than for other tests in use for this infection/disease with more foundational objective data concerning its utility and (33) The samples used for aspects of this testing come from samples of a unique cohort with analyses said to add more objective understanding of this infection since the parasite was discovered and to be a national and international resource” than other tests for this infection with the testing of such samples with metadata that likely cannot be obtained again in the future and (34) with testing approach considered by FDA formal comments to indicate acceptable predicate (but different from this novel test) and comparator tests, with a pathway outline to dual 510 k clearance and and CLIA waiver so it can be used to help patients in the future and (35) can be used as a comparator for future test development in the USA and (36) b—this is a test that fulfills ALL the WHO ASSURED (Affordable, Sensitive, Specific, Robust, Equipment free, Deliverable to users) criteria for persons who are infected due to exposure to T. gondii parasites of the many haplogroups in persons of varying genetics in 7 countries on 5 continents and multiple island nations and states including Canada, the USA mainland, Hawaii, Mexico, Panama, Colombia, French Guiana, Guadeloupe, Brazil, Philippines, multiple locations in France, Morocco and New Zealand in more than 6000 persons' samples, in many varied demographics, documented in studies with multiple testers and types of testers, in multiple settings including automobile drive by, home outdoor, physician offices, nurses' offices, employee health, phlebotomy laboratory, research laboratory, clinical laboratory, conference room, and office waiting areas, and other settings, AND with line data identifying those persons as immunocompetent or immune compromised having eye or brain disease, being a mother of a congenitally infected child, being pregnant or not. (FIGS. 1 to 17) and
c- this is a test that has demonstrated reproducibility in accordance with USA FDA written requirements, and absence of interference by multiple possible concomitant pathogens including chlamydia, gonococcus, leishmania, hepatitis A, hepatitis B, hepatitis C, influenza, syphilis, malaria, and other viruses such as Epstein Barr virus and
d—this is a test that can detect, using whole blood or serum, false positive commercially available tests in a USA clinical practice setting. Use of this test is thereby capable of limiting anxiety and concern for patients and their health care providers when caring for patients, whereas reference laboratory testing requires multiple days and major costs to complete confirmatory evaluation. This test can thereby, without delay, promptly detect the potential need for, and initiation of emergency life-, sight and cognition-saving treatment and
e- is a test that is useful in a “global initiative” to build public health programs that save lives, sight, motor function and cognition, where the efficacy and high performance is documented in multiple demographics with parasites of multiple divergent genetic strains, and has been documented to be well received in multiple settings by multiple testers and also those tested and
f- is a test that is useful to in a variety of clinical programs that can create circumstances in monthly obstetrical screening programs where there is spillover benefit for diagnosis of other associated diseases such as gestational diabetes and
g—is a test that is documented to be useful to detect infection in male and female. persons infected more than 50 years earlier in the USA, as well as those acutely infected and
h—this is a test that outperforms other lateral immunodiffusion tests produced in Ireland and in the USA and in several other countries on several continents and
i—this is a test which successfully detects seropositivity during seroconversion in acute infection as well as chronic infection and recognizes seronegative persons on multiple continents and countries when compared with comparator settings in persons in their own countries, easy to understand its use in multiple settings by testers with multiple skill sets in all these countries and
j—creation of a novel and revolutionary care paradigm in multiple countries saving life, sight, and cognitive functions and optimizing care as recognized by peer reviewers and those in patient care and news media to be of substantial benefit to help people, documented in a variety of public media and
k—useful in studies of seroprevalence, vaccine and medicine efficacy determinations and
l—useful in studies of wildlife in the USA and in epidemics even with pandemic potential and bioterrorism potential and
m—Can be used at scale with 1000 pregnant women in 4 regions of Morocco in a monthly screening program in gestation in nomadic populations, towns, and cities to compare uptake and accessibility and
n—has utility in evaluating the diagnosis of the following clinical problems: adenopathy, retinal disease in which Toxoplasma is the most frequent cause of infection in the back of the eye, seroconversion in gestation, infection pre transplantation and/or immune suppressive treatment, neurologic disease, illness manifested in utero, in the neonatal setting, although it does not distinguish maternal or child's antibodies, or persisting later in infancy, dating infection during in vitro fertilization and
o—the universal novel test that includes documentation of each and any of the above properties and functionalities which will save the lives and well-being of members of hundreds of thousands of families saving large amounts of money as documented by cost benefit analyses for individuals and governments while preventing lifelong morbidity and suffering and
p—can be used in conjunction with other tests that further define clinical status of infection such as apolipoprotein A being elevated in acutely infected persons, low in severely involved congenitally infected children, antibody to sporozoite protein indicative of mode of infection and duration of infection. (see FIG. 18)

TABLE 3B
Table B. Study 2 Part 1 Lyon Reference Laboratory ICT Test Results for Sera from Pregnant Patients
Referred by Local Physicians for T. gondii IgM with Predicate Tests in Local Laboratories and
Negative Western Blot as Gold Standard Comparator. The organization and data correspond to FIG. 2B.

| | | Gestation | | | | | University |
|---|---|---|---|---|---|---|---|
| | | when | | | | | hospital Lyon la |
| Patient | Sampling | tested | ICT | | | WB IgM | Croix Rousse |
| number | date | (week) | results | IgM | Test kit | results | conclusion |
| 1 | Sep. 8, 2021 | unknown | Negative | 3.84 | Cobas | Negative | Negative |
| 2 | Sep. 9, 2021 | 33 | Negative | 0.876 | Cobas | Negative | Negative |
| 3 | Dec. 28, 2021 | 34 | Negative | 3.26 | Cobas | Negative | Negative |
| 4 | Apr. 2, 2022 | 9 | Negative | 1.35 | Cobas | Negative | Negative |
| 5 | Dec. 20, 2021 | 32 | Negative | 1.19 | Roche Immunoelectro | Negative | Negative |
| 6 | Feb. 23, 2022 | 7 | Negative | Equivocal | Roche | Negative | Negative |
| | | | | | Electrochimiluminescence | | |
| 7 | Aug. 24, 2021 | 4 | Negative | 0.74 | Abbot Alinity | Negative | Negative |
| 8 | Sep. 20, 2021 | 4 | Negative | 1.3 | Roche Imimonoelectro | Negative | Negative |
| 9 | Nov. 23, 2021 | unknown | Negative | 0.54 | Abbot Alinity | Negative | Negative |
| 10 | Dec. 30, 2021 | 11 | Negative | 0.98 | Cobas | Negative | Negative |
| 11 | Jan. 13, 2022 | 23 | Negative | 1.45 | Roche Immunoelectro | Negative | Negative |
| 12 | Sep. 16, 2021 | unknown | Negative | Positive | Cobas | Negative | Negative |
| 13 | Sep. 14, 2021 | 16 | Negative | Positive | Cobas | Negative | Negative |
| 14 | Apr. 8, 2021 | 11 | Negative | 0.72 | Abbot Alinity | Negative | Negative |
| 15 | Oct. 12, 2021 | 36 | Negative | 0.92 | Abbot Alinity | Negative | Negative |
| 16 | Feb. 17, 2022 | 9 | Negative | 1.44 | Roche Immunoelectro | Negative | Negative |
| 17 | Oct. 26, 2021 | 15 | Negative | 1.11 | Centaur | Negative | Negative |
| 18 | Oct. 15, 2021 | 15 | Negative | 1.68 | Cobas | Negative | Negative |
| 19 | Oct. 21, 2021 | 14 | Negative | 1 | Centaur | Negative | Negative |
| 20 | Oct. 27, 2021 | 10 | Negative | 0.867 | Cobas | Negative | Negative |
| 21 | Sep. 22, 2021 | 7 | Negative | 11.67 | Roche | Negative | Negative |
| 22 | Jul. 28, 2021 | 32 | Negative | 0.66 | Abbot Alinity | Negative | Negative |
| 23 | Aug. 11, 2021 | 10 | Negative | Positive | Cobas | Negative | Negative |
| 24 | Sep. 8, 2021 | unknown | Negative | 1.48 | Cobas | Negative | Negative |
| 25 | Aug. 20, 2021 | 3 | Negative | 0.6 | Abbot Alinity | Negative | Negative |
| 26 | Apr. 17, 2021 | 30 | Negative | 0.6 | Architect | Negative | Negative |
| 27 | Nov. 23, 2021 | given birth | Negative | 0.9 | Cobas | Negative | Negative |
| 28 | Aug. 3, 2021 | 3 | Negative | 4.14 | Cobas | Negative | Negative |
| 29 | Jul. 10, 2021 | post birth | Negative | 2.68 | Centaur | Negative | Negative |
| 30 | Oct. 13, 2021 | 17 | Negative | 1.01 | Abbot Alinity | Negative | Negative |
| 31 | May 18, 2021 | 3 | Negative | 1.35 | Roche | Negative | Negative |
| 32 | Oct. 26, 2021 | 29 | Negative | Equivocal | Roche | Negative | Negative |

TABLE 3C
Table C. Study 3 Shows Concordance of ICT Results In Chicago Acceptability of Monthly Testing
In Testing of Sera in Lyon Reference Laboratory Using Abbott Architect and for one person
VIDAS IgG ELISA, and VIDAS G and M ELFA in Quindio Reference Laboratory. Initial tests in
earlier months were all concordant with Abbott Architect and reported in Lykins et al [28].
This study corresponds to FIG. 4 which shows the results of USA Acceptability, Study 3.

| | | | | | France Reference Laboratory | | | | | | |
|---|---|---|---|---|---|---|---|---|---|---|---|
| Colombia Reference Laboratory | | | | | | | [symbol] | | | | |
| | [symbol] IgG | | [symbol] IgM | | Architect | | western | VIDAS | | | |
| Patient | VIDAS | | VIDAS | | IgG | | blot | [symbol] G | VIDAS | Architect | |
| Code | [symbol] | Result | [symbol] | Result | [symbol] | Result * | Result | [symbol] | Result | IgM index | Result |
| 1-4 | 0 | Neg | 0.05 | Neg | 0.4 | Neg | | | | 0.05 | Neg |
| 1-5 | 0 | Neg | 0.2 | Neg | 0.5 | Neg | | | | 0.05 | Neg |
| 1-6 | 0 | Neg | 0.06 | Neg | 0.6 | Neg | | | | 0.06 | Neg |
| 1-7 | 0 | Neg | 0.05 | Neg | 0.5 | Neg | | | | 0.05 | Neg |
| 1-8 | 0 | Neg | 0.05 | Neg | 0.7 | Neg | | | | 0.07 | Neg |
| 2-4 | N/A | | | | 0.2 | Neg | | | | 0.05 | Neg |
| 2-5 | N/A | | | | 0.5 | Neg | | | | 0.07 | Neg |
| 2-6 | 0 | Neg | 0.04 | Neg | 0.5 | Neg | | | | 0.07 | Neg |
| 2-7 | 0 | Neg | 0.04 | Neg | 0.5 | Neg | | | | 0.04 | Neg |
| 2-8 | 0 | Neg | 0.04 | Neg | 0.2 | Neg | | | | 0.06 | Neg |
| 2-9 | 0 | Neg | 0.04 | Neg | 0.3 | Neg | | | | 0.03 | Neg |
| 3-4 | 0 | Neg | 0.04 | Neg | 2.8 | Eq | | 0 | Neg | 0.05 | Neg |
| 3-5 | 0 | Neg | 0.04 | Neg | 2.2 | Eq | | 0 | Neg | 0.06 | Neg |
| 3-6 | 0 | Neg | 0.04 | Neg | 2.1 | Eq | Neg | 56 | IQ | 0.06 | Neg |
| 3-7 | 0 | Neg | 0.04 | Neg | 2.9 | Eq | | 0 | Neg | 0.05 | Neg |
| 3-8 | 0 | Neg | 0.05 | Neg | 7.2 | Pos | | 0 | Neg | 0.03 | Neg |
| 4-4 | 0 | Neg | 0.04 | Neg | 0.1 | Neg | | | | 0.06 | Neg |
| 4-5 | 0 | Neg | 0.04 | Neg | 0.1 | Neg | | | | 0.03 | Neg |
| 4-6 | 0 | Neg | 0.04 | Neg | 0.1 | Neg | | | | 0.03 | Neg |
| 4-7 | 0 | Neg | 0.04 | Neg | 0.1 | Neg | | | | 0.03 | Neg |
| 5-4 | 0 | Neg | 0.05 | Neg | 0.3 | Neg | | | | 0.04 | Neg |
| 5-5 | 0 | Neg | 0.05 | Neg | IQ | | Neg | | | IQ | |
| 5-6 | 0 | Neg | 0.05 | Neg | 0.3 | Neg | | | | 0.04 | Neg |
| 5-7 | 0 | Neg | 0.04 | Neg | 0.4 | Neg | | | | 0.07 | Neg |
| 5-8 | 0 | Neg | 0.14 | Neg | 0.4 | Neg | | | | 0.04 | Neg |
| 6-4 | 2 | Neg | 0.05 | Neg | 0.2 | Neg | | | | 0.04 | Neg |
| 6-5 | 2 | Neg | 0.05 | Neg | 0.3 | Neg | | | | 0.04 | Neg |
| 6-6 | 2 | Neg | 0.05 | Neg | 0.2 | Neg | | | | 0.06 | Neg |
| 6-7 | 2 | Neg | 0.04 | Neg | 0.2 | Neg | | | | 0.07 | Neg |
| 7-4 | 0 | Neg | 0.04 | Neg | 0.4 | Neg | | | | 0.06 | Neg |
| 7-5 | N/A | | | | 0.3 | Neg | | | | 0.08 | Neg |
| 7-6 | 0 | Neg | 0.04 | Neg | 0.5 | Neg | | | | 0.04 | Neg |
| 7-7 | 0 | Neg | 0.03 | Neg | 0.4 | Neg | | | | 0.03 | Neg |
| 7-8 | 0 | Neg | 0.03 | Neg | 0.5 | Neg | | | | 0.05 | Neg |
| 8-4 | 0 | Neg | 0.05 | Neg | IQ | | Neg | | | 0.05 | Neg |
| 8-5 | 0 | Neg | 0.06 | Neg | 0.4 | Neg | | | | 0.07 | Neg |
| 8-6 | 0 | Neg | 0.06 | Neg | 0.4 | Neg | | | | 0.04 | Neg |
| 8-7 | 0 | Neg | 0.06 | Neg | 0.3 | Neg | | | | 0.07 | Neg |
| 9-4 | 0 | Neg | 0.08 | Neg | IQ | | Neg | | | IQ | |
| 9-5 | 0 | Neg | 0.03 | Neg | IQ | | Neg | | | 0.03 | Neg |
| 9-6 | 0 | Neg | 0.04 | Neg | 0.4 | Neg | | | | 0.05 | Neg |
| 9-7 | 0 | Neg | 0.12 | Neg | 0.2 | Neg | | | | 0.05 | Neg |
| 9-8 | 0 | Neg | 0.03 | Neg | 0.5 | Neg | | | | 0.04 | Neg |
| 10-4 | 0 | Neg | 0.06 | Neg | IQ | | Neg | | | 0.06 | Neg |
| 10-5 | 0 | Neg | 0.06 | Neg | 0.4 | Neg | | | | 0.05 | Neg |
| 10-6 | 0 | Neg | 0.06 | Neg | IQ | | Neg | | | 0.05 | Neg |
| 10-7 | 0 | Neg | 0.06 | Neg | 0.2 | Neg | | | | 0.04 | Neg |
| 10-8 | 0 | Neg | 0.08 | Neg | 0.3 | Neg | | | | 0.03 | Neg |
| 11-4 | 0 | Neg | 0.07 | Neg | 0.2 | Neg | | | | 0.04 | Neg |
| 11-5 | 0 | Neg | 0.07 | Neg | 0.4 | Neg | | | | 0.05 | Neg |
| 11-6 | 0 | Neg | 0.07 | Neg | 0.2 | Neg | | | | 0.04 | Neg |
| 11-7 | 0 | Neg | 0.05 | Neg | IQ | | Neg | | | 0.03 | Neg |
| 11-8 | N/A | N/A | N/A | N/A | 0.2 | Neg | | | | 0.06 | Neg |
| 12-4 | 0 | Neg | 0.04 | Neg | IQ | | Neg | | | 0.07 | Neg |
| 12-5 | 0 | Neg | 0.04 | Neg | IQ | | Neg | | | IQ | |
| 12-6 | 0 | Neg | 0.04 | Neg | 0.2 | Neg | | | | 0.07 | Neg |
| 12-7 | 0 | Neg | 0.04 | Neg | 0.5 | Neg | | | | 0.07 | Neg |
| 13-4 | 0 | Neg | 0.05 | Neg | 0.3 | Neg | | | | 0.04 | Neg |
| 13-5 | 0 | Neg | 0.04 | Neg | IQ | | Neg | | | 0.03 | Neg |
| 13-6 | 0 | Neg | 0.05 | Neg | IQ | | Neg | | | 0.05 | Neg |
| 13-7 | 0 | Neg | 0.06 | Neg | 0.2 | Neg | | | | 0.05 | Neg |
| 13-8 | 0 | Neg | 0.05 | Neg | 0.2 | Neg | | | | 0.06 | Neg |
| 13-9 | 0 | Neg | 0.03 | Neg | 0.3 | Neg | | | | 0.06 | Neg |
| 14-4 | 0 | Neg | 0.03 | Neg | 0.1 | Neg | | | | 0.03 | Neg |
| 14-5 | 0 | Neg | 0.05 | Neg | IQ | | Neg | | | 0.06 | Neg |
| 14-6 | 0 | Neg | 0.04 | Neg | IQ | | Neg | | | 0.03 | Neg |
| 14-7 | 0 | Neg | 0.04 | Neg | IQ | | Neg | | | 0.05 | Neg |
| 14-8 | 0 | Neg | 0.16 | Neg | 0.2 | Neg | | | | 0.02 | Neg |
| 15-4 | 0 | Neg | 0.04 | Neg | 0.3 | Neg | | | | 0.07 | Neg |
| 15-5 | 0 | Neg | 0.03 | Neg | IQ | | Neg | | | 0.04 | Neg |
| 15-6 | 0 | Neg | 0.04 | Neg | 0.2 | Neg | | | | 0.07 | Neg |
| 15-7 | 0 | Neg | 0.03 | Neg | 0.2 | Neg | | | | 0.04 | Neg |
| 15-8 | N/A | N/A | N/A | N/A | 0.2 | Neg | | | | 0.07 | Neg |
| 16-4 | 0 | Neg | 0.05 | Neg | 0.1 | Neg | | | | 0.07 | Neg |
| 16-5 | 0 | Neg | 0.05 | Neg | IQ | | Neg | | | IQ | |
| 16-6 | 0 | Neg | 0.04 | Neg | 0.2 | Neg | | | | 0.08 | Neg |
| 16-7 | 0 | Neg | 0.05 | Neg | 0.2 | Neg | | | | 0.08 | Neg |
| 16-8 | 0 | Neg | 0.05 | Neg | 0.1 | Neg | | | | 0.07 | Neg |
| 16-9 | 0 | Neg | 0.04 | Neg | 0.1 | Neg | | | | 0.07 | Neg |
| 17-4 | 0 | Neg | 0.02 | Neg | 0.3 | Neg | | | | 0.07 | Neg |
| 17-5 | 0 | Neg | 0.02 | Neg | 0.4 | Neg | | | | 0.07 | Neg |
| 17-6 | 0 | Neg | 0.02 | Neg | 0.5 | Neg | | | | 0.03 | Neg |
| 17-7 | 0 | Neg | 0.06 | Neg | IQ | | Neg | | | IQ | |
| 17-8 | 0 | Neg | 0.02 | Neg | IQ | | Neg | | | IQ | |
| 18-4 | 1 | Neg | 0.03 | Neg | 0.2 | Neg | | | | 0.02 | Neg |
| 18-5 | 0 | Neg | 0.04 | Neg | 0.3 | Neg | | | | 0.06 | Neg |
| 18-6 | 0 | Neg | 0.03 | Neg | 0.3 | Neg | | | | 0.03 | Neg |
| 18-7 | 0 | Neg | 0.03 | Neg | 0.3 | Neg | | | | 0.06 | Neg |
| 19-4 | 0 | Neg | 0.04 | Neg | 0.2 | Neg | | | | 0.04 | Neg |
| 19-5 | 0 | Neg | 0.05 | Neg | 0.3 | Neg | | | | 0.07 | Neg |
| 19-6 | 0 | Neg | 0.05 | Neg | 0.1 | Neg | | | | 0.07 | Neg |
| 19-7 | 0 | Neg | 0.05 | Neg | 0.2 | Neg | | | | 0.04 | Neg |
| 19-8 | 0 | Neg | 0.05 | Neg | 0.2 | Neg | | | | 0.05 | Neg |

[symbol]

indicates data missing or illegible when filed

The ICT test provided herein in various embodiments provides advantages and solutions to problems and can achieve improved outcomes. ICT can be used, for example, in a toolbox with curative medicines and preventative and therapeutic vaccines, and provide opportunities to understand and repair damage done from Toxoplasma infection and in work to eliminate this infection, optimize care, and use in health initiatives to protect domestic and wild animals. In various embodiments the test can detect seropositivity during seroconversion in acute infection as well as in chronic infection and recognize seronegative persons on multiple continents and countries. It is easy to understand and implement in international contexts, across different populations, different clinical settings, genders, and dates of infection.

The ICT test provided herein can detect using whole blood, or serum, false positives in other commercially available tests. The accuracy and point of care capacity of the test provides the advantage of limiting anxiety and concern for health care providers and patients. The test provides demonstrated reproducibility in accordance with regulatory requirements and absence of interferences by multiple concomitant pathogens, including, for example, Chlamydia, gonococcus, Leishmania, hepatitis A, hepatitis B, hepatitis C, influenza, syphilis, malaria, and other viruses such as Epstein Barr virus. The test meets WHO ASSURED Affordable, Sensitive, Specific, Robust, Equipment free, Deliverable to users) criteria for persons who are infected due to exposure to T. gondii parasites of the many haplogroups in persons of varying genetics and countries in thousands of persons (over 6000 persons' samples) in wide variety of settings.

What is claimed is:

1. A method for diagnosing and treating a Toxoplasma infection in a subject at risk of a Toxoplasma infection, comprising:
(a) adding about 20 to 30 μL of a whole blood sample from the subject to a sample well of a lateral flow immunochromatography cassette comprising:
(i) a nitrocellulose strip comprising a Toxoplasma antigen immobilized on a test band;
(ii) and a fiberglass conjugate pad in fluid communication with the nitrocellulose strip; wherein the conjugate pad is impregnated with black test latex particles coupled with the Toxoplasma antigen, and wherein the sample well is in fluid communication with the conjugate pad;
wherein adding the whole blood sample to the sample well promotes binding of anti-Toxoplasma antibodies in the whole blood sample to the Toxoplasma antigen coupled to the black test latex particles in the conjugate pad to form complexes comprising anti-Toxoplasma antibodies bound to the Toxoplasma antigen coupled to the black test latex particles;
(b) dispensing an eluting solution to the sample well and allowing migration of the complexes comprising anti-Toxoplasma antibodies bound to the Toxoplasma antigen coupled to the black test latex particles for about 20-30 minutes to promote binding of the complexes to the Toxoplasma antigen immobilized on the test band;
(c) detecting a black line at the test band thus indicating the subject has a Toxoplasma infection, therefore identifying the subject as diagnosed with a Toxoplasma infection; and
(d) administering an effective treatment to the subject for treating the Toxoplasma infection when the black line is detected at the test band.

2. The method of claim 1, wherein about 30 μL of the whole blood sample is added to the sample well.

3. The method of claim 1, wherein the whole blood sample is obtained from a finger prick.

4. The method of claim 1, wherein the nitrocellulose strip further comprises control latex particles that are optically distinguishable from the black test latex particles, and wherein the conjugate pad of further comprises control antigens immobilized on a control band.

5. The method of claim 4, wherein the control latex particles are blue.

6. The method of claim 4, wherein the control latex particles are impregnated with goat anti-rabbit antibodies, and wherein the control antigens comprise rabbit gamma immunoglobulins.

7. The method of claim 1, wherein the Toxoplasma infection is the result of an infection by Toxoplasma gondii.

8. The method of claim 7, wherein the Toxoplasma infection is the result of an infection by Toxoplasma gondii in North America, Central America, South America, Hawaii, Caribbean Islands, France, Eastern Europe, North Africa, New Zealand, and Philippines.

9. The method of claim 1, wherein the method has a sensitivity of at least about 90%, at least about 91%, at least about 92%, at least about 93%, at least about 94%, at least about 95%, at least about 96%, at least about 97%, at least about 98%, or at least about 99%, and/or wherein the method has a specificity of at least about 90%, at least about 91%, at least about 92%, at least about 93%, at least about 94%, at least about 95%, at least about 96%, at least about 97%, at least about 98%, at least about 99% or at least about 100%.

10. The method of claim 1, wherein the effective treatment is selected from the group consisting of: pyrimethamine, sulfadiazine, sulfapyrizine, sulfamethazine, sulfamerizine, azithromycin, clarithromycin, atovaquone, dapsone, cotrimoxazole, and combinations thereof.

11. The method of claim 1, wherein the method does not have interference causing false positive test results from an infection or illness selected from HIV, hepatitis A, hepatitis B, hepatitis C, EBV, malaria, influenza, gonococcal infection, syphilis, Chlamydia, and various immune compromised states.

12. The method of claim 1, wherein the anti-Toxoplasma antibodies in the whole blood sample are IgG and/or IgM antibodies.

13. A kit comprising a lateral flow immunochromatography device for diagnosing a Toxoplasma infection in a subject wherein the kit comprises:
(a) the lateral flow immunochromatography device, wherein the device comprises a cassette comprising:
(i) a nitrocellulose strip on which are immobilized a Toxoplasma antigen in a test band and a control antigen in a control band;
(ii) a conjugate pad in fluid communication with the nitrocellulose strip impregnated with test latex particles coupled with a Toxoplasma antigen and control latex particles coupled with a control antigen; and
(iii) a sample well in fluid communication with the conjugate pad;
(b) eluting buffer.

14. The kit of claim 13, wherein the kit comprises a timer.

15. The kit of claim 13, wherein the eluting buffer is in a 3 mL dropper bottle.

16. The kit of claim 13, wherein the kit comprises:
(c) 60 lateral flow immunochromatography devices, wherein there are 6 bags, and each bag contain 10 devices and a desiccant packet;
(d) 3 dropper bottles with the eluting buffer, where each dropper bottle comprises 3 mL of eluting buffer; and
(e) instructions for use.

17. The kit of claim 13, wherein the kit comprises:
(f) lancets for fingertip whole blood sampling; and
(g) capillary tubes for collecting the whole blood sample.

18. The method of claim 1, further comprising diagnosing acute Toxoplasma infection in a subject by identifying level of apolipoprotein A in the sera of a subject, wherein an elevated level of Apolipoprotein A indicates a recently acquired infection in an adult.

19. The method of claim 18, wherein the subject is a pregnant woman.

20. The method of claim 1, further comprising diagnosing acute Toxoplasma infection in a subject by identifying level of apolipoprotein A in the sera of a subject, wherein a reduced level of Apolipoprotein A in an infant indicates a sever disease is present.
